(19) United States
(12) Patent Application Publication
(10) Pub. No.: US 2019/0255078 A1
(43) Pub. Date: Aug. 22, 2019

(54) TOPICAL COMPOSITIONS WITH STABLE SOLUBILIZED SELECTIVE RETINOIDS AND/OR TETRACYCLINE-CLASS ANTIBIOTICS

(71) Applicant: BioPharmX, Inc., San Jose, CA (US)

(72) Inventors: Xin CHEN, Palo Alto, CA (US); Mouhannad JUMAA, Foster City, CA (US); Kin F. CHAN, Los Gatos, CA (US); Maiko HERMSMEIER, San Jose, CA (US); Tanvee Vinod SAWANT, Sunnyvale, CA (US)

(21) Appl. No.: 16/280,971

(22) Filed: Feb. 20, 2019

Related U.S. Application Data
(60) Provisional application No. 62/726,921, filed on Sep. 4, 2018.
(60) Provisional application No. 62/633,053, filed on Feb. 20, 2018.

(51) Int. Cl. A61K 31/65; A61K 47/02; A61K 47/10; A61K 47/14; A61K 31/4436; A61P 29/00; A61K 31/192; A61K 9/00

(52) U.S. Cl. CPC A61K 31/65; A61K 47/02; A61K 47/10; A61K 9/0014; A61K 31/4436; A61P 29/00; A61K 31/192; A61K 47/14

(57) ABSTRACT

Topical compositions for dermatological use are described. The topical compositions generally comprise a selective retinoid, such as tazarotene or adapalene, and/or a tetracycline-class antibiotic, such as minocycline or doxycycline, and a monohydric aliphatic alcohol. The compositions may further comprise a divalent cation, such as magnesium, calcium, or zinc, and an antioxidant, such as a sulfite or a thiosulfate. The composition may further comprise a carboxylate ester solvent.

### CROSS REFERENCE TO RELATED APPLICATIONS

This application claims the benefit of U.S. Provisional Application No. 62/726,921, filed Sep. 4, 2018 and of U.S. Provisional Application No. 62/633,053, filed Feb. 20, 2018, each incorporated by reference herein in its entirety.

### TECHNICAL FIELD

This disclosure is directed to a stable topical composition or dosage form comprising a selective retinoid alone, a tetracycline-class antibiotic alone, or both a selective retinoid and a tetracycline-class antibiotic, and in which both the selective retinoid (if present) and tetracycline-class antibiotic (if present) is solubilized in the composition. More particularly, this disclosure relates to topical compositions or topical dosage forms comprising a selective retinoid, such as tazarotene or adapalene, a tetracycline-class antibiotic, such as minocycline or doxycycline, or a combination thereof, where the selective retinoid, tetracycline-class antibiotic, or both are stable and solubilized. The compositions are for treating various dermatological conditions and diseases.

### BACKGROUND

The pathogenesis of acne involves the interaction of four key factors: excess sebum production, hypercornification of the epidermis around a hair follicle (i.e., ductal hypercornification), proliferation of Propionibacterium acnes (P. acnes) bacteria in the sebaceous gland, and increased inflammation. The order of occurrence of these four factors is not well known because there are many interactions among these factors. For example, hormones trigger excess follicular keratinization and increased sebum production (due to an increase in sebocyte productivity and/or sebocyte number). Increased keratinization leads to clogged pores and a sebum-rich anaerobic environment, which creates a favorable environment for proliferation of P. acnes bacteria, which signals a local inflammatory response and further stimulates keratinocyte and sebum production.

Inflammation is involved in both the early and late stages of acne vulgaris. Recent studies have suggested that inflammatory events may occur early in the development of acne lesions (microcomedones), even before the initial hyperproliferative change. In these studies, the uninvolved skin from acne patients was found to contain elevated levels of CD3+ and CD4+ T cells in the perifollicular and papillary dermis and increased macrophage numbers similar to those seen in papules.

Excessive sebum production has been known to be one of the key factors in acne. One of the few medications proposed to reduce sebum and the only one currently being sold commercially in the U.S. and other countries is tretinoin. Studies have demonstrated that some retinoids, such as tretinoin, have sebosuppressive effects through the regulation of AMPK-SREBP-1 signaling pathways and that AMPK has a critical role in the sebosuppression in SEB-1 sebocytes. However, tretinoin does not have antibacterial properties.

While it may not be possible to clearly identify a single root cause of acne, clinical trials with various drugs have demonstrated that acne can be treated, to various levels of success, by addressing one or more of the four factors mentioned above. The paper “New Treatments and Therapeutic Strategies for Acne” by Thiboutot (Arch. Fam. Med. 2000; 9: 179-187) describes several of the known treatments for acne in more detail.

For example, retinoids have been in use in dermatology for more than 30 years. Retinoids increase the rate of skin cell turnover, which can have mixed results for clogging or unclogging pores. This increased turnover is commonly referred to as “skin purging.” Increased skin cell turnover can unclog pores, but excess cellular debris can also clog pores, leading to increases in acne. The exfoliated skin cells may be physically carried from one region of the skin to another, which can result in clogged pores and the proliferation of P. acnes bacteria. Excess sweating can exacerbate these effects by temporarily enlarging pores, thus exacerbating the ingress of bacteria and/or excess skin cells. These effects can, individually or in combination, increase the intensity of acne. Irritation from increased skin cell turnover can also lead to increased inflammation and irritation.

Retinoids, including many selective retinoids, such as tazarotene and adapalene, can also dry out and irritate skin. Many patients using these selective retinoids also use moisturizer to limit dryness. If moisturizer is not applied, biological feedback systems trigger the skin to produce extra sebum to reduce dryness, thus making acne worse. Frequently such effects last 3-6 weeks.

Oral retinoids such as isotretinoin (ACCUTANE or ROACCUTANE, F. Hoffmann-La Roche AG, Basel, Switzerland) can have significant side effects. Due to the incidence rate for congenital defects for pregnant patients and other side effects, ACCUTANE was temporarily removed from the market and the use of isotretinoin requires regular pregnancy tests and blood test for lipid levels, which is a significant cost and inconvenience for patients.

Topical delivery of drugs is a preferred method of drug delivery for the treatment of many dermatological conditions because topical delivery localizes treatment to the region of the body affected by disease. In contrast to oral, trans-buccal, or intravenous administration, topical delivery results in a lower systemic drug concentration. In topical delivery, a larger portion of the delivered drug reaches the skin where its action can be effective in treating a dermatological condition or disease. This reduces the risk of side effects associated with high levels of systemic exposure.

Many topical drug products include only a single drug. However, some can be used beneficially in combination. For example, topical retinoid compositions that have high enough dose or concentration to be effective without combination with another active ingredient also have significant side effect profiles, including dry skin, flaky skin, inflammation, irritation, and photosensitivity. DIFFERIN gel (0.3% adapalene gel, Galderma Laboratories, L.P., Fort Worth, Tex.) has several side effects with incidence rates of at least 1%: dry skin, skin discomfort, pruritus, desquamation, and sunburn. Topical tretinoin compositions (e.g., RETIN-A MICRO (0.1% tretinoin gel, Valeant Pharmaceuticals North America, LLC, Laval, Quebec, Canada)), can trigger irritant dermatitis, which can present as erythema, scaling, and burning sensation.

EPIDUO (Galderma Laboratories, L.P., Fort Worth, Tex.) is a topical composition that contains 0.1% adapalene and 2.5% benzoyl peroxide. Benzoyl Peroxide is not an antibiotic. The combination is very irritating with approximately 14% of the treatment population reporting adverse events in clinical studies. Additionally, benzoyl peroxide strongly bleaches fabrics that it contacts. This can be a problem, for example, for a patient who applies a composition containing benzoyl to the shoulders, back, or face, where contact with clothing occurs in varying degrees.

Tazarotene compositions have been approved as topical treatments for acne, psoriasis, and photo-damaged skin. Such formulations are commercially sold under the brand names TAZORAC, AVAGE, ZORAC, and FABIOR. Each of these formulations suffers from a significant side effect profile that includes reports of itchiness, dry and cracking skin, redness, and enhanced sensitivity to sunlight (“photosensitivity”). Improved tazarotene compositions have been proposed that include the addition of emollients, moisturizers, or other ingredients to reduce selected side effects. However, these proposed solutions typically merely mask the side effects rather than addressing their underlying causes.

Furthermore, such proposed solutions are frequently incompatible with commercial requirements for topical compositions. Topical compositions require a delivery system that maintains the drug in a form that is simultaneously stable, bioavailable when applied, and uniform in concentration. Additionally, the topical composition preferably has good organoleptic properties to encourage patient compliance and provide a pleasant patient experience.

In addition to having poor side effect profiles, compositions that include tazarotene as the sole drug are limited in their effectiveness or may require large amounts of drug to obtain effective treatment. Tazarotene alone only addresses two of the four factors of acne: excess sebum production and excess keratinization.

To address limitations with retinoids, one approach is to deliver tazarotene or adapalene in combination with an antibiotic where the antibiotic is administered as a separate oral composition. However, patient compliance can be a significant problem and patients may choose to administer either the antibiotic or the retinoid, but not both. To address this problem, a composition that combines an antibiotic (e.g. minocycline or doxycycline) with a retinoid (e.g. adapalene or tazarotene) into a single composition would be beneficial.

Antibiotic drugs, such as tetracycline-class drugs, have also been used in dermatology for treatment of bacterial and/or inflammatory causes of acne. In the treatment of acne, several antibiotic drugs have been used systemically via an oral route of administration. Examples include doxycycline, minocycline, tetracycline, and erythromycin. However, many potential side effects occur based on high-dosage, systemic use of the drug, which exposes the entire body to drugs rather than localizing treatment to where it is needed. As a result, the use of systemic antibiotic drugs increases the potential side effects, increases the risk of developing bacterial resistance, and limits the maximum recommended local dose in comparison to what would be able to be used for a more directed local delivery of the active ingredient.

One example is a minocycline product sold as SOLODYN (Valeant Pharmaceuticals North America, LLC, Laval, Quebec, Canada) approved as an oral treatment for acne. The oral minocycline SOLODYN has a recommended dosage of about 1 mg per kg of body weight. Side effects for SOLODYN tablets with an incidence rate of at least 5% include headache, fatigue, dizziness, and pruritus (i.e., itching). Other side effects include somnolence (i.e., drowsiness), urticarial (i.e., hives), and arthralgia (i.e., joint pain). High systemic levels of minocycline can harm an unborn child and may make oral intake inaccessible by pregnant women.

An alternative to an oral dosage forms is a topical dosage form. Topical formulations of drug, such as retinoids and antibiotics, suffer from significant limitations. Topical compositions require a delivery system that maintains the drug in a form that is simultaneously stable, soluble, bioavailable when applied, and uniform in concentration. Additionally, the topical composition preferably has good organoleptic properties to encourage patient compliance and provide a pleasant patient experience. In topical drug compositions, the choice of antibacterial agent can be important. Many topical active ingredients drive antibacterial activity that kills or inhibits growth of P. acnes bacteria, but few of these also address the inflammatory causes of acne. Several topical antibiotics have been tried and each has its limitations. Clindamycin, for example, has been effective as a topical antibiotic but has been associated with “diarrhea, abdominal pain, bloody diarrhea, and colitis” (Thiboutot, supra). Topical antibiotics, especially clindamycin and erythromycin, are reported to have “a slow onset of action and predictable emergence of antibiotic-resistant bacterial organisms” (Eichenfield, et al., Pediatrics, 2013; 131; S163-S186).

There is a need for topical compositions that simultaneously provide solubility, stability, enhanced bioavailability, and/or uniform concentration of drug, while also providing good organoleptic properties. There is a further need for topical compositions of a selective retinoid alone, a tetracycline-class antibiotic alone, or a combination thereof, that are effective in the treatment of dermatological conditions or diseases, such as acne, while maintaining the properties of the topical composition described above.

### BRIEF SUMMARY

The following aspects and embodiments thereof described and illustrated below are meant to be exemplary and illustrative, not limiting in scope.

In one aspect, provided is a topical composition comprising a selective retinoid, a monohydric aliphatic alcohol, and a carboxylate ester with a ratio of ester groups to carbon atoms of at least 0.05.

In another aspect, provided is a topical composition comprising a tetracycline-class drug, a monohydric aliphatic alcohol, and a carboxylate ester with a ratio of ester groups to carbon atoms of at least 0.05. In some embodiments, the tetracycline-class drug is dissolved in the composition. In some embodiments, the tetracycline-class drug is stable when stored in a sealed glass container for at least 3 months at 40° C. or for at least 6 months at 20-25° C., optionally in a dark environment.

In another aspect, provided is a topical composition comprising a selective retinoid, a tetracycline-class drug, a monohydric aliphatic alcohol, and a carboxylate ester with a ratio of ester groups to carbon atoms of at least 0.05. In some embodiments, the selective retinoid and the tetracycline-class drug are dissolved in the composition. In some embodiments, the tetracycline-class drug is stable when stored in a sealed glass container for at least 3 months at 40° C. or for at least 6 months at 20-25° C., optionally in a dark environment.

In another aspect, provided is a topical composition comprising (i) a selective retinoid, a tetracycline-class drug, or a selective retinoid and a tetracycline class drug, (ii) a monohydric aliphatic alcohol, and (iii) a carboxylate ester with a ratio of ester groups to carbon atoms of at least 0.05. In some embodiments, the selective retinoid and/or the tetracycline-class drug, if present, are dissolved in the composition. In some embodiments, the tetracycline-class drug is stable when stored in a sealed glass container for at least 3 months at 40° C. or for at least 6 months at 20-25° C., optionally in a dark environment.

In some embodiments, the topical composition further comprises a salt of a divalent cation.

In another aspect, provided is a topical composition comprising a selective retinoid, a monohydric aliphatic alcohol, a salt of a divalent cation, and an antioxidant. In some embodiments, the topical composition further comprises a tetracycline-class drug. In embodiments, the molar ratio of the divalent cation to the tetracycline-class drug in the topical composition is at least 1:1, 2:1, 3:1 or 4:1, or is between about 0.75:1 to about 8:1, 0.75:1 to about 6:1, 0.75:1 to about 5:1, 1:1 to about 8:1, 1:1 to about 6:1, 1:1 to about 5:1 or 1:1 to about 4:1. In some embodiments, the topical composition further comprises a carboxylate ester with a ratio of ester groups to carbon atoms of at least about 0.05.

In a further aspect, provided is a topical composition comprising a tetracycline-class drug, a monohydric aliphatic alcohol, a salt of a divalent cation, and an antioxidant. In some embodiments, the molar ratio of the divalent cation to the tetracycline-class drug in the topical composition is at least about 4:1. In some embodiments, the topical composition further comprises a carboxylate ester with a ratio of ester groups to carbon atoms of at least 0.05.

In some embodiments, the concentration of the carboxylate ester in the topical compositions is between about 1% to 30% by weight. In some embodiments, the concentration of the carboxylate ester in the topical compositions is between about 5% to 20% by weight. In some embodiments, the carboxylate ester has a ratio of the number of ester groups to the number of carbon atoms of between about 0.05-0.30, 0.05-0.25, 0.05-0.20, or 0.05-0.15. In some embodiments, the carboxylate ester has a ratio of the number of ester groups to the number of carbon atoms of at least about 0.05. In some embodiments, the carboxylate ester has a ratio of the number of ester groups to the number of carbon atoms of at least about 0.10. In some embodiments, the carboxylate ester has a ratio of the number of ester groups to the number of carbon atoms of at least about 0.15. In some embodiments, the carboxylate ester has a ratio of the number of ester groups to the number of carbon atoms of at least about 0.20. In some embodiments, the carboxylate ester has a ratio of the number of ester groups to the number of carbon atoms of at least about 0.25. In some embodiments, the carboxylate ester has a ratio of the number of ester groups to the number of carbon atoms of at least about 0.30. In some embodiments, the carboxylate ester is selected from the group consisting of one or more of isopropyl myristate, medium-chain triglycerides, diisopropyl adipate, ethyl acetate, triacetin, dimethyl succinate, propyl acetate, and combinations thereof.

In some embodiments, the monohydric aliphatic alcohol is anhydrous. In some embodiments, the anhydrous monohydric aliphatic alcohol comprises less than about 0.005% water (200 proof), or less than about 5% water (190 proof), or less than about 2% water.

In some embodiments, the salt of a divalent cation is a magnesium salt. In some embodiments, the magnesium salt is anhydrous. In some embodiments, the magnesium salt is magnesium chloride, magnesium sulfite or magnesium thiosulfate. In some embodiments, the magnesium salt is anhydrous. In some embodiments, the magnesium salt is anhydrous magnesium chloride, anhydrous magnesium sulfite or anhydrous magnesium thiosulfate.

In some embodiments the monohydric aliphatic alcohol is selected from the group consisting of one or more of ethanol, isopropanol, propyl alcohol, tert-butyl alcohol, and combinations thereof. In some embodiments, the monohydric aliphatic alcohol is ethanol. In some embodiments, the monohydric aliphatic alcohol is volatile. In some embodiments, the concentration of the monohydric aliphatic alcohol in the topical composition is 50% to 99% by weight. In some embodiments, the concentration of the monohydric aliphatic alcohol in the topical composition is 60% to 80% by weight. In some embodiments, the concentration of the monohydric aliphatic alcohol in the topical composition is 70% to 95% by weight.

In another aspect, a topical composition for treatment or prophylaxis of a dermatological condition or disease is provided that comprises a selective retinoid, a tetracycline-class drug or a combination thereof. Typically, the selective retinoid, the tetracycline-class drug, or each is dissolved in the composition and/or the selective tetracycline-class drug and the retinoid are each stable when stored in a sealed glass container for at least about 3 months at 40° C. or for at least about 6 months at about 20-25° C. In other embodiments, the composition is stable at the noted time and conditions in a dark environment.

In some embodiments, the topical composition further comprises a divalent cation compound. In embodiments, the molar ratio of the divalent cation in the compound to the tetracycline class drug in the topical composition is at least about 1:1, 2:1, 3:1, or 4:1. In some embodiments, the salt of a divalent cation is a magnesium salt. In some embodiments, the magnesium salt is magnesium chloride, magnesium sulfite or magnesium thiosulfate.

In some embodiments, the tetracycline-class drug is minocycline or doxycycline. In embodiments, the tetracycline-class drug is minocycline. In some embodiments, the tetracycline-class drug is doxycycline.

In some embodiments, the selective retinoid is dissolved in the composition. In some embodiments, the selective retinoid is stable when stored in a sealed glass container for at least about 6 months at 20-25° C., optionally in a dark environment. In embodiments, the selective retinoid is tazarotene or adapalene. In some embodiments, the selective retinoid is tazarotene. In some embodiments, the selective retinoid is adapalene.

In some embodiments, for compositions comprising both a selective retinoid and a tetracycline-class antibiotic, the two drugs may be physically separated in different compartments within the container closure, with each drug mixed in a composition comprising of a divalent cation and one or more antioxidants. In some embodiments, the divalent cation is a magnesium salt. In some embodiments, the antioxidants may be a sulfite salt and or a thiosulfate salt.

In some embodiments, the selective retinoid and tetracycline-class drugs may be physically separated in different compartments within the container closure, with the tetracycline-class drug mixed in a composition comprising a divalent cation and one or more antioxidants. In some embodiments, the divalent cation is a magnesium salt. In embodiments, the antioxidants may be a sulfite salt and/or a thiosulfate salt.

In some embodiments, the water content of the composition is less than 5% as measured by Karl Fischer titration. In some embodiments, the water content of the composition is less than 2% as measured by Karl Fischer titration.

In some embodiments, the topical composition further comprises a polyol. In some embodiments, the polyol is a C3-C8 diol or a triol. In some embodiments, the polyol is propylene glycol. In some embodiments, the polyol is glycerol or glycerin. In some embodiments, the concentration of the polyol in the topical composition is 2% to 40% by weight. In some embodiments, the polyol is anhydrous.

In some embodiments, the topical composition further comprises a sulfite, a thiosulfate or a combination thereof. In some embodiments, the sulfite is sodium bisulfite, sodium metabisulfite, magnesium sulfite, or a combination thereof. In some embodiments, the thiosulfate is sodium thiosulfate, potassium thiosulfate, ammonium thiosulfate, magnesium thiosulfate or a combination thereof.

In some embodiments, the topical composition comprises the salt of a divalent cation and anti-oxidant are one compound that provides both a divalent cation and anti-oxidant properties, such as, for example, magnesium sulfite or magnesium thiosulfate.

In some embodiments, the topical composition further comprises one or more pharmaceutically acceptable excipients selected from the group consisting of one or more of a thickener, an antioxidant, an emollient, a foam adjuvant, a foam propellant, a foam adjuvant, and a surfactant.

In some embodiments, the topical composition is not an emulsion. In some embodiments, the topical composition does not comprise propyl ethylene glycol. In embodiments, the topical composition does not comprise glycerol. In some embodiments, the topical composition does not comprise glycerin. In some embodiments, the topical composition does not comprise a foam propellant or foam adjuvant.

In another aspect, a method for treatment of a dermatological condition or disease is provided. The method comprises topically applying a topical composition as described herein to an exterior epithelial surface of a mammalian body at least once daily for a period of at least 4 weeks.

In another aspect, a method for treatment of a dermatological condition or disease comprises topically applying a topical composition comprising a selective retinoid, a tetracycline class antibiotic, or a combination thereof, a monohydric aliphatic alcohol, and a carboxylate ester, wherein the carboxylate ester comprises ester groups and carbon atoms and the ratio of the number of ester groups to the number of carbon atoms is at least 0.10, to an exterior epithelial surface of a mammalian body at least once daily for a period of at least 4 weeks. In some embodiments, the dermatological condition or disease is acne or rosacea. In other embodiments, the dermatological condition or disease is psoriasis. In some embodiments, the dermatological condition or disease is an orphan disease, such as hidradenitis suppurativa (acne inversa), Hailey-Hailey (familial benign pemphigus) and/or Darier's disease (keratosis follicularis).

In still another aspect, a method for treatment of a dermatological condition or disease comprises topically applying a topical composition comprising tazarotene, a monohydric aliphatic alcohol, and a carboxylate ester, wherein the carboxylate ester comprises ester groups and carbon atoms and the ratio of the number of ester groups to the number of carbon atoms is at least 0.10, to an exterior epithelial surface of a mammalian body at least once daily for a period of at least 4 weeks. In some embodiments, the dermatological condition or disease is acne or rosacea. In other embodiments, the dermatological condition or disease is psoriasis. In some embodiments, the dermatological condition or disease is an orphan disease, such as hidradenitis suppurativa (acne inversa), Hailey-Hailey (familial benign pemphigus) and/or Darier's disease (keratosis follicularis).

In still another aspect, a method for treatment of a dermatological condition or disease comprises topically applying a topical composition comprising minocycline, a monohydric aliphatic alcohol, and a carboxylate ester, wherein the carboxylate ester comprises ester groups and carbon atoms and the ratio of the number of ester groups to the number of carbon atoms is at least 0.10, to an exterior epithelial surface of a mammalian body at least once daily for a period of at least 4 weeks. In some embodiments, the dermatological condition or disease is acne or rosacea. In other embodiments, the dermatological condition or disease is psoriasis. In some embodiments, the dermatological condition or disease is an orphan disease, such as hidradenitis suppurativa (acne inversa), Hailey-Hailey (familial benign pemphigus) and/or Darier's disease (keratosis follicularis).

In still another aspect, a method for treatment of a dermatological condition or disease comprises topically applying a topical composition comprising a selective retinoid, such as tazarotene or adapalene, and a tetracycline-class drug, such as minocycline or doxycycline, a monohydric aliphatic alcohol, and a carboxylate ester, wherein the carboxylate ester comprises ester groups and carbon atoms and the ratio of the number of ester groups to the number of carbon atoms at least about 0.05, 0.10, 0.15, 0.2 or is between 0.05-0.30, to an exterior epithelial surface of a mammalian body at least once daily for a period of at least 4 weeks. In some embodiments, the dermatological condition or disease is acne or rosacea. In other embodiments, the dermatological condition or disease is psoriasis. In some embodiments, the dermatological condition or disease is an orphan disease, such as hidradenitis suppurativa (acne inversa), Hailey-Hailey (familial benign pemphigus) and/or Darier's disease (keratosis follicularis).

In yet another aspect, a method for making a topical composition comprises (i) providing a selective retinoid and/or a tetracycline class antibiotic, a magnesium salt, a sulfite compound, or a thiosulfate compound or a combination thereof, a monohydric aliphatic alcohol, a polyol, and a carboxylate ester to form a mixture, and (ii) agitating the mixture from (i) to form a solution in which the drug or drugs are dissolved and the composition comprises less than 2% water content as measured by Karl Fischer titration.

Additional embodiments of the composition, related methods, components of the composition, and the like will be apparent from the following description, examples, figures and claims. These and other objects and features of the disclosure will become more fully apparent when read in conjunction with the following detailed description.

### BRIEF DESCRIPTION OF THE DRAWINGS

FIG. 1 is a graph illustrating the average penetration efficiency of minocycline and of tazarotene into ex vivo human facial skin as a function of the ratio of ester groups to carbon atoms in the carboxylate ester following application of compositions each comprising minocycline hydrochloride, tazarotene, magnesium chloride (anhydrous), ethanol (anhydrous), hydroxypropyl cellulose HF, a polyol, and a selected carboxylate ester as described in Example 1.

FIG. 2 is a graph illustrating the solubility of tazarotene in compositions comprising ethanol and selected polyols and carboxylate esters as described in Example 2. Mixtures comprising carboxylate esters and ethanol show unexpectedly enhanced solubility. In contrast, binary mixtures of ethanol and a polyol (e.g. propylene glycol or glycerin) show predictable solubility characteristics for a binary mixture.

FIG. 3A is a graph illustrating the stability of tazarotene in solution in a solvent comprising ethanol and propylene glycol as described in Example 3A. The stability of tazarotene when the solution further comprises magnesium chloride and sodium metabisulfite (SMBS), individually or in combination, is also presented to illustrate the effects of these components on stability.

FIG. 3B is a bar graph showing the stability of minocycline in solution in a solvent comprising ethanol and propylene glycol as described in Example 3B. The stability of minocycline when the solution further comprises magnesium chloride and sodium metabisulfite, individually or in combination, is also presented to illustrate the effects of these components on stability.

FIG. 4 is a graph illustrating the average uptake of tazarotene into ex vivo human facial skin for four different compositions and for an untreated control. TAZORAC 0.05% cream and 0.1% gel are compared to exemplary compositions as described in Example 4, which comprise 0.05% and 0.03% tazarotene, respectively. Uptake of tazarotene with the exemplary embodiments is more efficient than uptake for the TAZORAC compositions.

FIGS. 5A-5B are graphs showing the stability of tazarotene (FIG. 5A) and minocycline (FIG. 5B) in compositions as described in Example 5 when stored at room temperature for 6 months, 12 months, and 18 months.

FIGS. 6A-6B are graphs showing the uptake of tazarotene (FIG. 6A) and minocycline (FIG. 6B) into ex vivo human facial skin for compositions of Table 9 according to the study of Example 6.

FIGS. 7A-7D are graphs showing the uptake of tazarotene (FIGS. 7A, 7C) and minocycline (FIGS. 7B, 7D) into ex vivo human facial skin for compositions as described in Tables 13-1 and 13-2 of Example 9.

FIGS. 8A-8B are graphs showing the stability of tazarotene (FIG. 8A) and minocycline (FIG. 8B) in compositions as described in Table 13-1 when stored at 40° C. for 2 weeks, 1 month, 3 months, and 6 months.

FIGS. 9A-9B are graphs showing the stability of minocycline in compositions as described in Table 21 and Example 15, where FIG. 9A shows the stability of minocycline compositions when stored at 40° C. for 3 months and FIG. 9B shows the stability of minocycline compositions when stored at 40° C. for 6 months.

FIG. 10 is a bar graph showing the amount of minocycline, tazarotene and tazarotenic acid, a metabolite, that penetrated in vivo into rat skin after once daily application for 14 days from compositions identified as combo C.30.3 and combo C.28.1 (See Table 13-1 in Example 9).

FIGS. 11A-11C show results of an in vivo study with Sprague Dawley rats, where FIG. 11A shows the amount of tazarotene in skin following topical application of a commercially marketed topical tazarotene composition (TAZORAC gel 0.05%) and a tazarotene composition identified as C30.3 (see Table 13-1 of Example 9) and FIGS. 11B-11C, respectively, show the erythema and dryness scores for the compositions.

### DETAILED DESCRIPTION

The present invention will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art. As can be appreciated from the foregoing and following description, each and every feature described herein, and each and every combination of two or more of such features, is included within the scope of the present disclosure provided that the features included in that such combinations are not inconsistent. In addition, any feature or combination of features may be specifically excluded from any embodiment of the present invention. Additional aspects and advantages of the present invention are set forth in the following description and claims, particularly when considered in conjunction with the accompanying examples and drawings.

All publications, patents and patent applications cited herein are hereby incorporated by reference in their entirety, unless otherwise indicated. In an instance in which the same term is defined both in a publication, patent, or patent application incorporated herein by reference and in the present disclosure, the definition in the present disclosure represents the controlling definition. For publications, patents, and patent applications referenced for their description of a particular type of compound, chemistry, etc., portions pertaining to such compounds, chemistry, etc. are those portions of the document which are incorporated herein by reference.

### Definitions

It must be noted that, as used in this specification, the singular forms “a,” “an,” and “the” include plural referents unless the context clearly dictates otherwise. Thus, for example, reference to an “active ingredient” includes a single ingredient as well as two or more different ingredients, reference to a “solvent” refers to a single solvent as well as to two or more different solvents, reference to a “magnesium salt” includes a single magnesium salt as well as two or more different magnesium salts, and the like.

In describing and claiming the present invention, the following terminology will be used in accordance with the definitions described below.

The term “topical composition” refers to a material that comprises pharmaceutically acceptable ingredients, including an active ingredient, and is intended for administration to an animal or human subject and is applied to the surface of the skin, in contrast to materials that are taken orally or through intravenous injection. Topical compositions are typically administered for the purpose of alleviation of symptoms associated with a dermatological disease or condition, treatment of a dermatological disease or condition, and/or prevention of a dermatological disease or condition.

The term “treatment of a dermatological condition or disease” refers to alleviation of symptoms associated with a dermatological condition or disease, treatment of a dermatological condition or disease, prophylaxis of a dermatological condition or disease, and/or prevention of a dermatological condition or disease.

As used herein, “dermatological condition” refers to cosmetic and pathological disorders of the skin. Dermatological conditions include topical inflammatory skin conditions such as eczema, seborrhoeic dermatitis, bullous dermatoses, cutaneous sarcoidosis, Kaposi's sarcoma, neutrophilic dermatoses, contact dermatitis, rosacea, psoriasis, hidradenitis suppurativa, acne including acne rosacea, acne vulgaris, and orphan diseases such as hidradenitis suppurativa, Hailey-Hailey, Darier's disease, and infections such as impetigo, cellulitis, erysipelas, folliculitis, furuncles, carbuncles, Lyme disease, and other skin infections.

As used herein, “acne” is a disorder of the skin characterized by papules, pustules, cysts, nodules, comedones, and other blemishes or skin lesions. These blemishes and lesions are often accompanied by inflammation of the skin glands and pilosebaceous follicles, as well as, microbial, especially bacterial, infection. As used herein, acne includes all known types of acne. Some types of acne include, for example, acne vulgaris, cystic acne, acne inversa, acne atrophica, bromide acne, chlorine acne, acne conglobate, acne cosmetics, acne detergicans, epidemic acne, acne estivalis, acne fulminans, halogen acne, acne indurata, iodide acne, acne keloid, acne mechanica, acne papulosa, pomade acne, premenstral acne, acne pustulosa, acne scorbutica, acne scrofulosorum, acne urticata, acne varioliformis, acne venenata, propionic acne, acne excoriee, gram negative acne, steroid acne, nodulocystic acne and acne rosacea. Acne rosacea is characterized by inflammatory lesions (erythema) and telangiectasia. Telangiectasia is abnormally and permanently dilated blood vessels associated with a number of diseases. For example, facial telangiectasia is associated with age, acne rosacea, sun exposure, and alcohol use.

A solvent is said to “dissolve” a drug if the solubility for that drug at 25° C. and atmospheric pressure is greater than the concentration of the drug in the solvent. For emulsions and the like, the drug is only considered to “dissolve” in the solvent if the drug is in direct interaction with the solvent. So, for example, a drug that is coated to limit interaction with a solvent would not be considered dissolved in that solvent if it remained in particulate form.

The term “solvent” refers to a substance in which one or more solid ingredients are dissolved to some extent. For clarity, the solid ingredient does not need to be fully dissolved (i.e. as defined above) in the substance for it to be considered a solvent. For example, ethanol, isopropanol, and propylene glycol are solvents, to name a few, for minocycline.

A monohydric aliphatic alcohol or polyol is “anhydrous” if it comprises less than 1% water content as measured by Karl Fischer titration. For example, anhydrous ethanol (or, equivalently “ethanol (anhydrous)” or “ethanol, anhydrous”) means ethanol that comprises less than 1% water content as measured by Karl Fischer titration.

Anhydrous magnesium chloride (or, equivalently “magnesium chloride (anhydrous)” or “magnesium chloride, anhydrous”) means magnesium chloride that comprises less than 5% water content as measured by Karl Fischer titration.

The term “carboxylate ester” is a compound that comprises an ester of the form

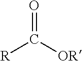

where R and R′ are any alkyl groups and the compound comprises at least 1 carbon atom. R′ cannot be a hydrogen atom. Either R and R′ can be an ester. Examples of carboxylate esters include cyclic esters of hydroxycarboxylic acids, isopropyl myristate, diisopropyl adipate, dimethyl succinate, and propyl acetate.

The terms “drug,” “active ingredient,” and “active pharmaceutical ingredient” are used interchangeably herein.

The term “tetracycline-class drug” refers to tetracycline and tetracycline derivatives such as minocycline, doxycycline, oxytetracycline, and their corresponding pharmaceutically acceptable salt forms, as well as solvates and hydrates thereof. A tetracycline antibiotic generally contains a four ring octahydrotetracene-2-carboxamide skeleton, while the actual substituents on the skeleton may vary.

The term “tetracycline” refers to (4S,4aS,5aS,6S,12aR)-4-(dimethylamino)-1,6,10,11,12a-pentahydroxy-6-methyl-3,12-dioxo-4,4a,5,5a-tetrahydrotetracene-2-carboxamide (i.e., CAS number 60-54-8) and its corresponding pharmaceutically acceptable salt forms, as well as solvates and hydrates thereof. For example, a common salt form of tetracycline is tetracycline HCl (i.e., CAS number 64-75-5).

The term “minocycline” refers to (4S,4aS,5aR,12aR)-4,7-bis(dimethylamino)-1,10,11,12a-tetrahydroxy-3,12-dioxo-4a,5,5a,6-tetrahydro-4H-tetracene-2-carboxamide (i.e. CAS number 10118-90-8) and its corresponding pharmaceutically acceptable salt forms, as well as solvates and hydrates thereof. Exemplary forms of minocycline are commonly identified by their CAS numbers. For example, minocycline hydrochloride has a CAS number of 13614-98-7.

The term “doxycycline” refers to (4S,4aR,5S,5aR,6R,12aS)-4-(dimethylamino)-3,5,10,12,12a-pentahydroxy-6-methyl-1,11-dioxo-1,4,4a,5,5a,6,11,12a-octahydrotetracene-2-carboxamide (i.e., CAS number 564-25-0) and its corresponding pharmaceutically acceptable salt forms, as well as solvates and hydrates thereof.

The term “retinoid” refers to chemical compounds that activate retinoic acid receptors and/or retinoid X receptors. Examples of retinoids include, without limitation, tretinoin, isotretinoin, tazarotene, adapalene, bexarotene, calcipotriene, etretinate, and alitretinoin and their corresponding pharmaceutically acceptable salt forms, as well as solvates and hydrates thereof.

The term “selective retinoid” refers to a retinoid that activates one or more retinoic acid receptors (RARs), such as RAR-alpha, RAR-beta, or RAR-gamma, and does not significantly activate retinoid X receptors (RXRs). Adapalene and tazarotene are examples of selective retinoids because they are retinoids that selectively activate RAR-beta and RAR-gamma and do not significantly activate RXRs.

The term “adapalene” refers to 6-[3-(1-adamantyl)-4-methoxy-phenyl]naphthalene-2-carboxylic acid (i.e., CAS number 106685-40-9) and its derivatives and their corresponding pharmaceutically acceptable salt forms, as well as solvates and hydrates thereof.

The term “tazarotene” refers to ethyl 6-[2-(4,4-dimethyl-2,3-dihydrothiochromen-6-yl)ethynyl]pyridine-3-carboxylate (i.e., CAS number 118292-40-3) and its derivatives and their corresponding pharmaceutically acceptable salt forms, as well as solvates and hydrates thereof.

The term “monohydric aliphatic alcohol” refers to a monofunctional organic compound that contains a single hydroxyl group, in which the hydroxyl functional group is covalently attached to a saturated carbon atom forming part of a branched or linear alkyl chain, and which does not contain an aromatic-ring configuration of atoms. Generally, a monohydric aliphatic alcohol for use in the compositions provided herein conforms to the formula R—OH, where R is a C₁-C₄ alkyl. Suitable R groups include ethyl, propyl, isopropyl, butyl, sec-butyl, isobutyl and tert-butyl.

The term “polyol” refers to a pharmaceutically acceptable alcohol containing two or more hydroxyl groups, and possessing from 3-8 carbon atoms. Polyols suitable for use in the instant compositions may, but do not necessarily, contain functional groups in addition to the hydroxyl groups, such as e.g., an ether bond. As used herein, polyethylene glycol shall not be considered to be a polyol. Illustrative polyols include diols such as propylene glycol (PG) and dipropylene glycol, triols such as glycerol, 1,2,6 hexanetriol, trimethylolpropane, and higher alcohols (i.e., containing more than 3 hydroxyl groups) such as sorbitol and pentaerythritol. Polyols also include butylene glycol, hexylene glycol, 1,6 hexanediol, mannitol, and xylitol. It is recognized that some of these solvents are solids that may be undesirable, but when combined in appropriate mixtures, they may be suitable for use in a topical composition as described herein.

The term “topical” refers to application to an exterior epithelial surface of the body, including the skin or cornea. For purposes of this application, applications inside a bodily orifice, such as the mouth, nose, or ear shall not be considered to be topical applications.

A drug is said to be “stabilized” by the presence of a particular material contained in a composition if a composition comprising all of the same materials in the same relative proportions to each other, excluding the active ingredient or drug, but with the particular material removed, exhibits a loss in potency that is greater than the loss of potency for the original composition when stored at 20° C. to 25° C. in a dark environment in a sealed glass container for 6-months. For clarity, when performing the replacement (i.e., assessment of stability enhancement), the weight percentage of the drug in the topical composition is not increased, but instead the removed material is effectively replaced by equivalent proportions from the rest of the topical composition excluding the drug. For example, if a composition containing 30% (w/w) A, 30% (w/w) B, 30% (w/w) C, and 10% (w/w) D is evaluated for the effect of component A, and D is the active ingredient (i.e., tetracycline-class drug), the comparative composition will contain 0% (w/w) A (the excluded component), 45% (w/w) B, 45% (w/w) C, and 10% (w/w) D (the active ingredient).

A drug is said to be “stable” in a composition over a specified test period and under specified storage conditions if the potency of the drug is maintained at a therapeutic level that is 90% to 110% of the potency of the drug at the beginning of the test period. As used herein, if not specified, the time period for evaluating whether a drug is “stable” in a composition is 6 months. As used herein, if not specified, the storage conditions for evaluating whether a drug is “stable” in a composition are that the composition is stored in a sealed glass container at 20° C. to 25° C. in a dark environment.

A solvent or composition is said to be “volatile” if it has a vapor pressure of 35 mm mercury at a pressure of 1 atmosphere and a temperature of 30° C.

The abbreviation “(w/w)” indicates that relative concentrations of a composition are presented on a “weight for weight” basis (i.e. percentages refer to a percentage of the total weight), rather than on the basis of volume.

The term “pharmaceutically acceptable” in reference to an entity or ingredient is one that may be included in the compositions provided herein and that causes no significant adverse toxicological effects in the patient at specified levels, or if levels are not specified, in levels known to be acceptable by those skilled in the art. All ingredients in the compositions described herein are provided at levels that are pharmaceutically acceptable. For clarity, active ingredients may cause one or more side effects and inclusion of the ingredients with a side effect profile that is acceptable from a regulatory perspective for such ingredients will be deemed to be “pharmaceutically acceptable” levels of those ingredients.

“Pharmaceutically acceptable salt” denotes a salt form of a drug or active ingredient, or other ingredient having at least one group suitable for salt formation that causes no significant adverse toxicological effects to the patient. Reference to an active or other ingredient as provided herein is meant to encompass its pharmaceutically acceptable salts, as well as solvates and hydrates thereof. Pharmaceutically acceptable salts include salts prepared by reaction with an inorganic acid, an organic acid, a basic amino acid, or an acidic amino acid, depending upon the nature of the functional group(s) in the drug. Suitable pharmaceutically acceptable salts include acid addition salts which may, for example, be formed by mixing a solution of a basic drug with a solution of an acid capable of forming a pharmaceutically acceptable salt form of the basic drug, such as hydrochloric acid, iodic acid, fumaric acid, maleic acid, succinic acid, acetic acid, citric acid, tartaric acid, carbonic acid, phosphoric acid, sulfuric acid and the like. Typical anions for basic drugs, when in protonated form, include chloride, sulfate, bromide, mesylate, maleate, citrate and phosphate. Suitable pharmaceutically acceptable salt forms and methods for identifying such salts are found in, e.g., Handbook of Pharmaceutical Salts: Properties, Selection and Use, Weinheim/Zürich:Wiley-VCH/VHCA, 2002; P. H. Stahl and C. G. Wermuth, Eds.

“Therapeutically effective amount” is used herein to mean the amount of a pharmaceutical preparation, or amount of an active ingredient in the pharmaceutical preparation, that is needed to provide a desired level of active ingredient in the bloodstream or in a target tissue. The precise amount will depend upon numerous factors, e.g., the particular active ingredient, the components and physical characteristics of the pharmaceutical preparation, intended patient population, patient considerations, and the like, and can readily be determined by one skilled in the art, based upon the information provided herein and available in the relevant literature.

The term “patient” refers to a living organism suffering from or prone to a condition that can be prevented or treated by administration of a composition as provided herein, and includes both humans and animals.

“Optional” or “optionally” means that the subsequently described circumstance may or may not occur, so that the description includes instances where the circumstance occurs and instances where it does not.

In many cases, the patent application describes ranges of values. Such ranges shall be construed to include the endpoints of the range unless doing so would be inconsistent with the text or otherwise noted.

### Overview

The present application provides a topical composition and related methods for using the topical composition for the treatment or prophylaxis of a dermatological condition or disease, for example, in the treatment or prophylaxis of acne, rosacea, hidradenitis suppurativa, or orphan indications such as hidradenitis suppurativa, Hailey-Hailey or Darier's disease. The topical composition comprises a selective retinoid and preferably, but optionally, a tetracycline-class drug. More preferably, the selective retinoid is tazarotene or adapalene and the tetracycline-class drug is minocycline or doxycycline, where present.

The instant disclosure addresses at least some of the problems previously identified herein, e.g., in the Background section, related to topical compositions comprising a retinoid or a tetracycline class drug. After several composition attempts, the Applicants have discovered a composition and related solvent system that promotes the penetration of a selective retinoid, and preferably a tetracycline class drug, into the skin and in which such drugs are both stable and have high solubility. In such a solvent system, the drugs ideally remain in solution for a significant portion of the time it takes for the solvent to penetrate into the skin. As has been recognized by the Applicants, if some of the solvent is lost to evaporation prior to penetration, the concentration of the drugs in the solvent on the skin surface will typically be increased, which means that the solubility of the drugs within the composition is an important feature for consideration in designing an improved topical composition comprising a selective retinoid and/or a tetracycline class drug. Additionally, as has been discovered by the Applicants, selecting a solvent in which the drugs are fully dissolved during storage reduces or eliminates variations in drug concentration throughout the composition. In the description that follows, minocycline is often referred to as the exemplary tetracycline class drug, however, the compositions and methods disclosed herein also apply to tetracycline-class drugs other than minocycline, such as, but not limited to, doxycycline. Similarly, in the description that follows, tazarotene is often referred to as the exemplary selective retinoid, however, the compositions and methods disclosed herein also apply to selective retinoids other than tazarotene, such as, but not limited to, adapalene. Additionally, a liquid composition comprising a selective retinoid, a tetracycline-class drug, or combinations thereof and having superior stability is also provided herein.

The present application provides a topical composition and related methods for preparing the topical composition.

In one aspect, the topical composition comprises tazarotene, a monohydric aliphatic alcohol, and a carboxylate ester, where details regarding the tazarotene (an exemplary selective retinoid), the monohydric aliphatic alcohol, and the carboxylate ester are provided above and in the sections which follow. It has been discovered that liquid compositions such as provided herein, e.g., comprising tazarotene in a solvent system comprising a monohydric aliphatic alcohol and a carboxylate ester, are stable and, when applied topically, the tazarotene penetrates well into human skin. See, e.g., Example 1. Surprisingly, the penetration is typically higher for compositions comprising carboxylate esters that have a larger ratio of ester groups to carbon atoms. As seen in Table 3, compositions comprising ethyl acetate (composition C.7.2) had a carboxylate ester with a ratio of ester groups to carbon atoms of 0.25. These compositions had a normalized minocycline uptake as high as 1.03, and a normalized tazarotene uptake as high as 1.19, which is 2-3 times larger than the penetration efficiencies of compositions comprising lower ratios (albeit still high ratios). Thus, compositions comprising a carboxylate ester with a larger ratio of ester groups to carbon atoms had an increased efficiency of penetration (seen by the normalized uptake) for tazarotene and minocycline when compared to compositions comprising a carboxylate ester having a lower ratio of ester groups to carbon atoms. From this data, it appears that using carboxylate esters having increasing ratios of ester groups to carbon atoms in a composition results in a corresponding increase in efficiency of penetration of tazarotene and/or minocycline.

A solvent system including carboxylate esters having increased ratios of ester groups to carbon atoms, such as ethyl acetate, has the benefit of efficient penetration of tazarotene and minocycline. It has been discovered that the use of certain similarly structured carboxylate esters (e.g., dimethyl succinate, propyl acetate, or combinations thereof) can be particularly effective for desirable usability characteristics of the topical composition. For example, dimethyl succinate, propyl acetate, or combinations thereof, have a less pungent smell than that of ethyl acetate, without compromising penetration, solubility, and stability of the compositions.

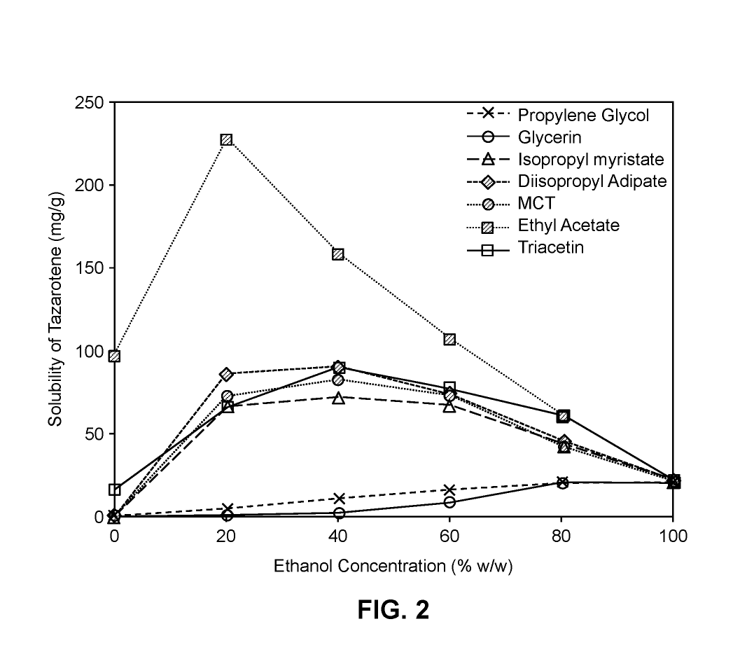

Applicants have further discovered a solvent system in which the drug (selective retinoid and/or tetracycline class antibiotic) is/are substantially completely dissolved and/or have increased solubility, when compared to the solubility of the tetracycline-class drug in the carboxylate ester alone. Table 5 provides the solubility of tazarotene (mg/g) in various compositions comprising a monohydric aliphatic alcohol and a carboxylate ester. As described in Example 2, compositions comprising a mixture of a monohydric aliphatic alcohol (e.g. ethanol) and a carboxylate ester (e.g. isopropyl myristate, diisopropyl adipate, and/or MCT) significantly improved the solubility of tazarotene in the composition. The combined solvent systems provided a solubility for tazarotene of at least 25 mg/g for selected ratios of carboxylate esters to monohydric aliphatic alcohol. By comparison, the solubility of tazarotene in ethanol is approximately 21 mg/g. As seen in FIG. 2 and Tables 4 and 5, compositions comprising ethanol and at least one carboxylate ester in binary and tertiary mixtures resulted in tazarotene solubility of at least about 2-5 times the solubility of tazarotene in ester alone. Also as seen in Tables 4 and 5, compositions comprising ethanol and at least one carboxylate ester in binary and tertiary mixtures resulted in minocycline solubility of at least about 100 times the solubility of minocycline in ester alone.

In another aspect, the topical composition comprises minocycline and/or tazarotene, a monohydric aliphatic alcohol, a magnesium salt, and sodium metabisulfite, where details regarding the tazarotene (an exemplary selective retinoid), the minocycline (an exemplary tetracycline-class antibiotic), the monohydric aliphatic alcohol, the magnesium salt (a representative salt of a divalent cation), and sodium metabisulfite (a representative antioxidant) are provided above and in the sections which follow. It has been discovered that liquid compositions such as provided herein, e.g., comprising tazarotene, minocycline or both, in a solvent system comprising a monohydric aliphatic alcohol, a divalent cation, and an antioxidant are stable. See, e.g., Examples 3A-3B. Such compositions may further include a carboxylate ester as described above.

In another aspect, the topical composition comprises tazarotene or adapalene, minocycline or doxycycline, a monohydric aliphatic alcohol, polyol, a magnesium salt, and a sulfite, a thiosulfate or both a magnesium sulfite and a thiosulfate, where details regarding the tazarotene or adapalene (exemplary selective retinoids), minocycline or doxycycline (exemplary tetracycline-class drugs), the monohydric aliphatic alcohol, and the carboxylate ester are provided above and in the sections which follow. Surprisingly, such compositions combining both a selective retinoid and a tetracycline-class drug are stable when the compositions further comprise magnesium salt, wherein the molar ratio of magnesium to the tetracycline-class drug is at least about 4:1, and a sulfite, such as sodium bisulfite or sodium metabisulfite. See, e.g., Example 5. In other embodiments, this ratio is from about 2:1 to 10:1, 2:1 to 8:1, 1:1 to 8:1, 1:1 to 6:1, 1:1 to 5:1, 2:1 to 5:1, at least 2:1, at least 3:1, at least 7:1, or at least 10:1.

In another aspect, the topical composition comprises minocycline or doxycycline, a monohydric aliphatic alcohol, polyol, a magnesium salt, and a sulfite and/or thiosulfate where details regarding the minocycline or doxycycline (exemplary tetracycline-class drugs), the monohydric aliphatic alcohol, and the carboxylate ester are provided above and in the sections, which follow. Such compositions are stable when the compositions further comprise magnesium salt, wherein the molar ratio of magnesium to the tetracycline-class drug is at least 4:1, and a sulfite, such as sodium bisulfite or sodium metabisulfite. Preferably, this ratio is 2:1 to 10:1, at least 2:1, at least 7:1, or at least 10:1.

In some embodiments, the composition may be used for the treatment of a dermatological condition or disease. Non-limiting examples of dermatological conditions or diseases for which the composition may be used include but are not limited to acne, rosacea, seborrhoeic dermatitis, psoriasis, hidradenitis suppurativa, Hailey-Hailey, Darier's disease, and superficial skin infections such as impetigo, as well as in wound management.

Several dermatological conditions and diseases, such as acne (e.g., acne vulgaris, acne conglobata, truncal acne, acne mechanica, and acne fulminans), rosacea, and gram-negative folliculitis, can result from the interaction of several key factors, including excess sebum production, ductal hypercornification, proliferation of P. acnes, and increased inflammation. The discussion below is generally with reference to methods of treating acne. However, it will be appreciated that the compositions and methods described below may be applied to any or all of the dermatological conditions and diseases as described herein.

Since there is an interaction among these four factors, the suppression of at least one or two of these is helpful in mitigating such dermatological conditions and diseases or treating mild to moderate cases. For example, suppressing sebum production reduces the availability of a suitable environment for P. acnes to proliferate, thus making it easier to reduce excess P. acnes colonies. Similarly, reducing ductal hypercornification unclogs pores such that the P. acnes do not have a suitable anaerobic environment for growth. Killing P. acnes populations reduces the triggers that cause a local inflammation. Reducing inflammation supports the reduction of ductal hypercornification by removing triggers of keratinocyte proliferation. Despite these interdependencies, these dermatological conditions and diseases are most effectively and efficiently managed by addressing two or more factors of acne, and preferably three or more factors, simultaneously to treat each of the root causes directly. This can lead to faster and more effective treatment.

By using a tetracycline-class drug alone or in combination with a selective retinoid, a lower dose of each can be used than would be required for a similar treatment if either were used separately. Using a lower dose of each can reduce the incidence or type of side effects while still maintaining an effective treatment. For example, reducing the dose of a selective retinoid may limit the thinning of the stratum corneum that is commonly observed with selective retinoids. Similarly, reducing the dose of a tetracycline-class drug may limit the development of bacterial resistance. In other embodiments, severe cases are not able to be treated with a selective retinoid or with a tetracycline-class antibiotic alone, but would be responsive to the combination.

Alternatively, by using a tetracycline-class drug in combination with a selective retinoid, a higher dose of the selective retinoid can be used than would be tolerable if the selective retinoid were used as the only drug. The tetracycline-class drug enables the use of a higher dose of the selective retinoid without a corresponding increase of side effects typically associated with the selective retinoid such as inflammation and irritation. Using a higher dose of the selective retinoid enables treatment or prophylaxis of dermatological conditions and diseases that would not be resolved by treatment with a tolerable dose of the selective retinoid or the tetracycline-class drug on its own. Thus, the two APIs work synergistically to enable a new treatment. In some embodiments, the preferred ratio of concentrations between the tetracycline-class drug and the selective retinoid is in the range of 2:1 to 250:1, 5:1 to 100:1, 10:1 to 40:1, 20:1 to 35:1, or 40:1 to 100:1. In embodiments, the ratio is about 2:1, 5:1, 10:1, 35:1, 40:1, 50:1, 100:1, 250:1 or any combination of these ranges.

Additionally, there are some dermatological conditions and diseases that are not caused by the four factors of acne that are also responsive to certain selective retinoids and/or tetracycline-class drug. For example, selective retinoids can be useful in the treatment of hidradenitis suppurativa, Hailey-Hailey, Darier's disease, psoriasis, actinic keratosis, scars, and pigmentation disorders. For example, tazarotene is useful for the treatment of psoriasis. For treatment of such dermatological conditions and diseases, a topical composition comprising a selective retinoid and/or a tetracycline-class drug can be used to improve the treatment. For such dermatological conditions and diseases, the selective retinoid treats the underlying condition or disease and the tetracycline-class drug reduces the side effect profile of the selective retinoid by limiting irritation, inflammation, and/or the potential for infection. By reducing these side effects, the tetracycline-class drug can also reduce the incidence rate and/or intensity of longer term side effects such as post-inflammatory hyperpigmentation (particularly in darker skin types) and/or post-inflammatory scarring.

One of the challenges to delivering a tetracycline-class drug and a selective retinoid is finding a formulation that maintains the stability of the tetracycline-class drug and the selective retinoid and delivers them efficiently into the skin. Applicants have discovered a topical composition that comprises at least two drugs (i.e., at least a tetracycline-class drug and a selective retinoid), addresses each of the four factors of acne simultaneously, and maintains the stability of the tetracycline-class drug and the selective retinoid. These two APIs work synergistically for treatment or prophylaxis of a dermatological condition or disease, while also limiting irritation, inflammation, and the potential for infection.

### Selective Retinoids

Selective retinoids address excess sebum production and excess keratinization causes of acne and other dermatological conditions and diseases. The choice of a selective retinoid is made based on the desired treatment profile and the target receptors. In some embodiments, the selective retinoid is adapalene, tazarotene, or combinations thereof.

Selective retinoids activate RARs, but do not significantly activate RXRs. These different receptors serve different functions within the body and thus have different treatment and side effect profiles. Many of the effects regulated by RARs relate to cell differentiation and proliferation, while many of the effects regulated by RXRs relate to apoptosis. Selective retinoids are preferred over other retinoids, such as tretinoin and isotretinoin, because their receptor selectivity produces beneficial mediation of cell differentiation and/or proliferation while limiting side effects caused by activation of RXRs.

Tazarotene, in particular, is a preferred selective retinoid for several reasons. Tazarotene is a selective retinoid that modulates cellular differentiation, desquamation (i.e., cell proliferation), and inflammation.

Alternatively, adapalene is also a preferred selective retinoid. In comparison to many other retinoids, adapalene has fewer side effects, has superior anti-inflammatory properties, is more stable in a topical composition, and/or has more rapid onset. Adapalene inhibits keratinocyte differentiation and proliferation, which reduces the formation of comedones. Adapalene reduces expression of both toll-like receptor 2 (TLR2) and IL-10. Adapalene increases cell turnover to reduce the clogging of pores and the formation of microcomedones.

Selection between preferred selective antibiotics can be made based on the desired efficacy and side effect profile. Tazarotene 0.1% was shown to be clinically more effective than adapalene 0.1% gel or cream and clinically more effective than tretinoin 0.1% microsphere gel. Anja Thielitz, et al., Topical retinoids in acne—an evidence-based overview, 6 J. German Soc'y of Derm., 1023, 1023 (2008). However, adapalene 0.1% gel was clinically better tolerated than tazarotene 0.1% gel. Id.

On their own, selective retinoids compositions are typically comedolytic and normalize both follicular hyperproliferation and hyperkeratinization. Topical selective retinoids thus reduce the numbers of microcomedones and comedones. However, selective retinoids also have significant side effects, such as thinning the stratum corneum or causing photosensitivity. Additionally, selective retinoids are irritating to the skin, in part due to inflammation and in part due to reducing the barrier function of the skin which causes drying and makes the skin prone to infection. The combination in a topical dosage form of a selective retinoid with a tetracycline-class drug can mitigate such side effects by reducing inflammation and/or eliminating infectious bacteria. Tetracycline-class drugs, by reducing inflammation caused by selective retinoid, also limit the enlargement of sebaceous glands, which can mitigate other side effects.

While selective retinoids alone can frequently beneficially treat mild to moderate acne, severe acne is often resistant to treatment with a selective retinoid alone due to a lack of potency for doses with acceptable side effects. Additionally, selective retinoids alone can also enlarge pores, thus allowing excess skin cells and infectious bacterial agents to enter into the pores. This may lead to a worsening of the symptoms of the dermatological condition or disease being treated. Such side effects may be temporary, lasting perhaps a few weeks, or last for the duration of the treatment. Combination with a tetracycline-class drug mitigates many of these side effects.

### Tetracycline-Class Drugs

Tetracycline-class drugs address bacterial and/or inflammatory causes of acne and other dermatological conditions and diseases. Tetracycline-class drugs are generally preferred over other antibiotics in the compositions and methods described herein. Tetracycline-class drugs are beneficially bacteriostatic instead of bactericidal, which reduces the potential for development of antibacterial resistant strains of bacteria. Tetracycline-class drugs, such as tetracycline, doxycycline, and minocycline, have been orally administered for decades for the treatment of acne vulgaris and have a well characterized side effect profile. The effectiveness of these agents appears to relate to a combination of antimicrobial effects, such as the ability to reduce P. acnes counts, and multiple anti-inflammatory properties.

Tetracycline class drugs include for example tetracycline and tetracycline derivatives such as demeclocycline, minocycline, doxycycline, oxytetracycline, and their corresponding pharmaceutically acceptable salt forms, as well as solvates and hydrates thereof. The tetracycline class drug may also be a fluorocycline, i.e., a 7-fluoro-9-substituted-6-demethyl-6-deoxytetracycline, such as eravacycline (TP-434) or 7-fluoro-9-pyrrolidinoacetamido-6-demethyl-6-deoxytetracycline and their corresponding pharmaceutically acceptable salt forms, as well as solvates and hydrates thereof. Drugs belonging to the tetracycline class generally contain a four ring octahydrotetracene-2-carboxamide skeleton, while the actual substituents on the skeleton may vary. Tetracyclines are broad spectrum antibiotics, exhibiting activity against a broad range of bacteria. One preferred tetracycline for use in the compositions provided herein is minocycline. Minocycline is a potent semi-synthetic tetracycline with activity against a wide range of gram-positive and gram-negative organisms. Minocycline presents a broader spectrum when compared to other tetracycline-type compounds, and is also the most lipid-soluble of the tetracyclines, i.e., can more readily penetrate into various tissues when compared to other tetracyclines. The compositions provided herein may contain minocycline or any or the tetracycline antibiotics in any available form, e.g., as the free base, as a hydrochloride or other pharmaceutically acceptable salt, including all crystalline polymorphs, solvates, hydrates, or amorphous forms thereof.

Of the tetracycline-class drugs, minocycline has numerous characteristics that beneficially distinguishes it from other tetracycline-class drugs. For example, one or more of the following characteristics may distinguish minocycline from other tetracycline-class drugs: 1) blocks the production of interleukin (IL)-like cytokines and inhibits P. acnes lipase enzyme, thus preventing the release of follicular-free fatty acids 2) suppresses production of chemotactic factors that attract neutrophils to the follicular site, 3) inhibits phagocytosis and subsequent release of proinflammatory enzymes, 4) inhibits neutrophil migration to the site of inflammation, 5) activates superoxide dismutase, which reduces the adverse impact of reactive oxygen species, 6) inhibits several cytokines, such as IL-1, IL-6, tumor necrosis factor-α, 7) inhibits growth of protein kinase C, which is associated with granuloma formation, 8) increases the levels of interleukin (IL)-10, IL-15 and vascular endothelial growth factor (VEGF) in the brain (IL-10 is anti-inflammatory, IL-15 can prevent apoptosis, and VEGF is neuro-protective), and 9) increases attachment to and modulation of the inflammatory and proliferative response of fibroblasts, thus serving a key role in wound healing and scar formation. Of the tetracycline-class drugs, several microbiologic studies have demonstrated that minocycline exhibits superior P. acnes reduction in comparison to tetracycline and doxycycline. Minocycline has also been shown to exhibit multiple anti-inflammatory properties, many of which are unrelated to its antimicrobial properties. Minocycline may also have fewer side effects related to photosensitivity in comparison to other tetracycline-class drugs.

Ross et al. demonstrated in a study of clinical isolates of antibiotic-resistant P. acnes that tetracycline-class drugs beneficially have low MIC values for such bacteria. The average MIC90 for minocycline (4 μg/mL) was half that observed for doxycycline (8 μg/mL); eight (8) times lower than that observed for tetracycline (32 μg/mL); 16 times lower than that observed for clindamycin (64 μg/mL); and at least 128 times less than that observed for erythromycin (˜512 μg/mL). (Ross J I, Snelling A M, Eady E A, et al., Br J Dermatol. 2001; 144: 339-346).

Minocycline, which has a Log P of about 0.05 (free base form), and doxycycline, which has a Log P of about −0.02 (free base form), are both more lipophilic than tetracycline, which has a log P of about −1.3 (free base form). The more lipophilic nature of minocycline and doxycycline is believed to result in their higher propensity for accumulation within the sebaceous gland and sebum. As a result, minocycline and doxycycline are better able to partition into lipid-rich target areas where P. acnes bacteria concentrations are typically highest.

On a cellular level, erythromycin, clindamycin, and tetracycline bind to the ribosomal RNA (rRNA) of P. acnes, blocking protein synthesis and disrupting crucial cellular processes. Resistance to these agents develops when point mutations take place within the bacterial rRNA, most likely interfering with or compromising attachment of these drugs to P. acnes ribosomal subunits. It has also been suggested that these products cause ribosomal conformational changes, which may interfere with or cause dissociation of drug binding to ribosomes.

For tetracycline-class drugs, a point mutation in 16S rRNA of the small ribosomal subunit is responsible for typical resistance for P. acnes bacteria. The reason that minocycline has demonstrated over time the lowest level of P. acnes resistance based on evaluation of MICs and prevalence data is not well understood. Assuming that an attachment to the ribosomal subunit of P. acnes is necessary for inhibition of protein synthesis, minocycline may possess molecular structural properties that are less susceptible to the P. acnes resistance response.

The compositions described herein are useful for treating dermatological diseases. Contemplated for treatment are include rosacea, psoriasis, hidradenitis suppurativa, Hailey-Hailey and Darier's disease. Rosacea symptoms include facial skin flushing, bumps and pimples, visible blood vessels and/or eye irritation. Although the cause of rosacea is not known, these characteristics can be attributed to immune system and neurovascular dysregulation. Increased presence of Demodex folliculorum mites and genetics may also be contributing factors to rosacea symptoms. Oral antibiotics like oral doxycycline and/or retinoids like isotretinoin have been prescribed to help reduce inflammation, lesions and prevent flare-ups.

Psoriasis is an autoimmune disease characterized by raised, inflamed, scaly red lesions on the skin caused by the rapid accumulation of skin cells on the skin surface. Like Rosacea, the cause of psoriasis is not known, but symptoms can result from the immune system, genetics, and/or environmental factors. As described above, tazarotene compositions have been approved as topical treatments for acne, psoriasis, and photo-damaged skin. Such formulations are commercially sold under the brand names TAZORAC, AVAGE, ZORAC, and FABIOR.

Hidradenitis suppurativa, also known as acne inversa, is an immune mediated disease that includes a range of symptoms associated with hair follicles such as comedones, breakouts, and/or abscesses under the skin which may progress into painful inflamed clusters (sinus tracts). Symptoms result from immune dysregulation, genetics, hormonal dysregulation, and/or environmental factors. Treatments include topical and oral antibiotics, such as topical clindamycin and oral minocycline, to help prevent disease progression, and oral retinoids.

Hailey-Hailey disease, also known as familial benign pemphigus, is a rare genetic disorder characterized by blistering skin rashes and cracked plaques, which are also prone to infection. A mutation in the ATP2C1 gene results in epidermal calcium dysregulation affecting keratinocyte desmosomes leading to suprabasilar acantholysis (epidermal blistering). Treatments include topical and oral antibiotics to clear bacterial infections, such as topical clindamycin and oral minocycline, and oral retinoids.

Darier's disease, also known as keratosis follicularis, is a rare genetic disorder characterized by wart-like and greasy plaques on the skin, which are prone to infection. In addition to skin, nails are also affected. A mutation in the ATP2A2 gene results in calcium dysregulation affecting keratinocyte desmosomes leading to acantholysis and abnormal keratinization forming horny bumps. Treatments include topical retinoids, such as adapalene or tazarotene, oral retinoids, and oral antibiotics to clear bacterial infections.

With regard to treating acne, tetracycline-class drugs are clinically effective for the treatment of some dermatological conditions and diseases at least because they reduce the number of P. acnes found on the skin surface and in the hair follicles. Such P. acnes can trigger inflammatory acne and inflammatory responses to acne. So, reducing their numbers reduces the clinical presentation of inflammatory acne and inflammatory responses to acne. However, a limitation of minocycline alone is that it works primarily for inflammatory lesions, pus-filled pimples, and papules. It works less well for non-inflammatory acne lesions.

In some embodiments, the amount of tetracycline class drug in the topical composition (e.g., minocycline) typically ranges from about 0.01% to about 10% by weight, or from about 0.1% to about 5% by weight. Illustrative ranges are from about 0.1% to about 4% by weight, or from about 0.2% to about 3% by weight or from about 0.2% to about 1.5% by weight. For example, the topical formulation may comprise any one of the following weight percentages of minocycline or other tetracycline class antibiotic: 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 3%, 4%, 5%, 6%, 7%, 8%, 9%, 10% and so forth.

### Compositions and Uses

In aspects, topical compositions and related methods for making the topical composition are provided. In some embodiments, the topical composition generally comprises a selective retinoid, such as tazarotene or adapalene, and/or a tetracycline class antibiotic, such as minocycline or doxycycline, a monohydric aliphatic alcohol, and a carboxylate ester with a ratio of ester groups to carbon atoms of at least 0.05. In some embodiments, the topical composition also includes one or more of the following: a polyol, a magnesium salt, a sulfite and/or a thiosulfate and excipients such as a thickener, an emollient, an antioxidant, a foam adjuvant, a foam propellant, and a surfactant. Preferably, the selective retinoid, if present, and, if present, minocycline or doxycycline, are dissolved within the composition.

Topical delivery of a drug, such as tazarotene, adapalene, doxycycline, or minocycline, does not require as large of a dose as the same drug administered orally in order to produce the same level of drug within the sebaceous gland, hair follicle, and/or skin. Thus, smaller doses applied topically can be as effective in controlling a dermatological condition or disease, but with fewer side effects and less likelihood of inducing resistance for a tetracycline-class drug.

Several exemplary compositions are described in the Examples. Some preferred embodiments of the topical composition contain one or more of ethanol, propylene glycol, cineole, sodium metabisulfite, and magnesium chloride. Anhydrous ethanol is an anhydrous volatile solvent. Propylene glycol is particularly useful for dissolving minocycline. Cineole is particularly useful for dissolving adapalene. Additionally, one or more antioxidants can be added, such as sodium metabisulfite. Magnesium chloride can optionally be added to stabilize the minocycline, especially in combination with a sulfite and/or a thiosulfate. Magnesium chloride is preferably anhydrous. Antioxidants and divalent cations can be also added from sources of compounds composed of antioxidants and divalent cations, such as magnesium sulfite, magnesium thiosulfate or the like. Example 7 describes some specific, but exemplary, topical compositions as embodied herein.

Exemplary compositions as provided herein may comprise from about 40% to 99% (w/w) monohydric aliphatic alcohol, from about 0.01% to 1.0% (w/w) selective retinoid, from about 0.1% to 10% (w/w) tetracycline class drug, from about 0.2% to 15% (w/w) magnesium and from about 0.05% to 15% (w/w) antioxidant. Some preferred compositions may further comprise about 2% to 40% (w/w) or about 5% to 40% (w/w) polyol, about 1% to 60% (w/w), about 1% to 30% w/w, or about 5% to 60% (w/w) carboxylate ester with a ratio of ester groups to carbon atoms of at least 0.05, or combinations thereof.

One of the challenges is formulating a topical composition in which the drug or drugs are soluble, stable and/or bioavailable. Tetracycline-class drugs can be partially stabilized through the use of selected divalent cations, such as Mg²⁺, Ca²⁺, and Zn²⁺. Preferably, the cation used with minocycline is Mg²⁺, which can be added to a topical composition as magnesium chloride anhydrous (CAS 7786-30-3, Spectrum Chemical Manufacturing Co., New Brunswick, N.J.) or other magnesium salt. Adding an antioxidant and/or a chelating agent may further stabilize the tetracycline-class drug and/or retinoid. Applicants have discovered that sulfite and thiosulfate antioxidants, and particularly sodium bisulfite, sodium metabisulfite, sodium sulfite, sodium thiosulfate, and combinations thereof, are particularly suited to stabilizing minocycline. Antioxidants and divalent cations can be added from some sources for divalent cations, some sources for antioxidant, or some sources of compounds composed of antioxidants and divalent cations, such as magnesium sulfite, magnesium thiosulfate or similar, or a combination thereof.

The topical composition may further comprise a source of magnesium such as a magnesium salt. Illustrative magnesium salts include but are not limited to magnesium bromide, magnesium chloride, magnesium fluoride, magnesium iodide, magnesium sulfate, magnesium salicylate, and magnesium phosphate, magnesium sulfite and magnesium thiosulfate. Magnesium salts are often supplied commercially as hydrates, and hydrates can be used in the instant formulations. However, in some preferred embodiments, the magnesium salt is anhydrous, due to the instability of tetracycline-class drugs such as minocycline in the presence of water. It will be appreciated that magnesium may be present in the resulting composition in any available form, e.g., as the cation or as a salt. The term “magnesium salt” as used herein refers to all such sources of magnesium. Similarly, the term “salt of a divalent cation” as used herein refers to a divalent cation, a salt of a divalent cation, or other forms of a divalent atom that would make the divalent cation available to interact chemically with other components of the composition. Magnesium salt in the composition is effective to increase the solubility of the tetracycline class drug. Typical concentrations of magnesium in the topical compositions provided herein range from about 0.2-10% by weight. Molar ratios of the divalent cation (e.g., magnesium salt) to the tetracycline class drug, e.g., minocycline or doxycycline, range from about 2:1 to about 100:1. Illustrative molar ratios are typically at least about, about, or between about any of the following ratios: 2:1 (Mg:tetracycline drug), 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1, 20:1, 30:1, 40:1, 50:1, 60:1, 75:1 and 100:1. The magnesium has been found to stabilize minocycline especially when combined with a sulfite and to stabilize tazarotene when combined with an antioxidant.

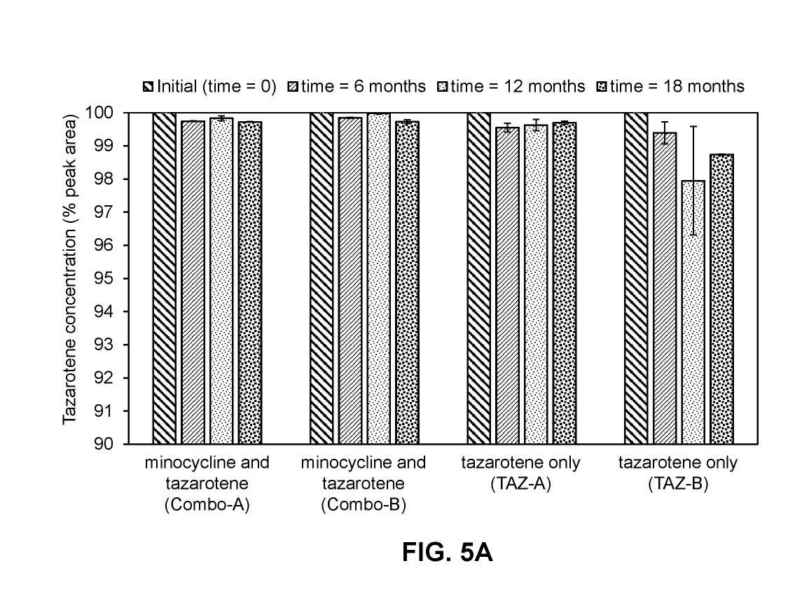

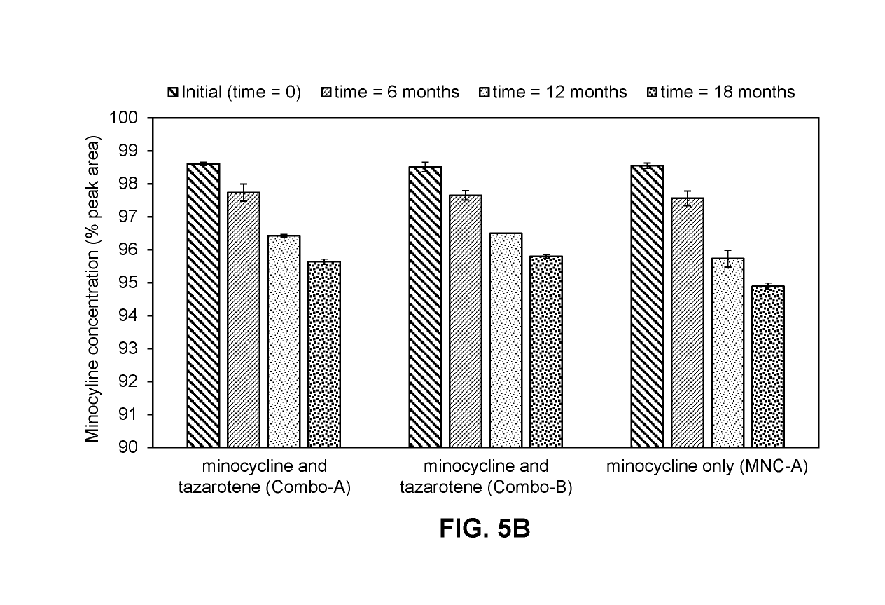

As described in Example 5, compositions comprising a selective retinoid and a tetracycline class antibiotic as described herein are storage stable for extended periods of time. Compositions as described herein including tazarotene and minocycline were stable after storage for at least about 6 months, or at least about 12 months, or at least about 18 months. As seen in FIGS. 5A-5B, the compositions comprising tazarotene and minocycline retained at least 90-95% (% peak area) after storage for 6 months, 12 months or 18 months. As seen in FIGS. 5A-5B, the compositions comprising tazarotene and minocycline in combination are more stable when compared to compositions containing tazarotene or minocycline as the sole active ingredient.

As described in Example 15, compositions comprising a tetracycline class antibiotic as described herein are storage stable for extended periods of time. Compositions as described herein including minocycline were stable after storage for at least 3 months. As seen in Table 21, the compositions comprising minocycline retained at least 85-95% (% peak area) after storage for 3 months and 6 months.

Alternatively, or in addition to a magnesium salt, the topical formulation may comprise a salt of a divalent metal cation such as, for example, calcium, aluminum, zinc, where illustrative counter-ions and relative amounts (e.g., for total divalent metal ion) are as described above for a magnesium salt. Preferred divalent metal ions are those capable of interacting with minocycline and/or those capable of stabilizing minocycline.

The topical composition generally additionally comprises, as part of its solvent system, a monohydric aliphatic alcohol, preferably a volatile alcohol. Generally, a monohydric aliphatic alcohol for use in the compositions provided herein conforms to the formula R—OH, where R is a C₁-C₄ alkyl group. Suitable R groups include methyl, ethyl, propyl, isopropyl, butyl, sec-butyl, isobutyl and tert-butyl. Preferably, the monohydric aliphatic alcohol is a primary alcohol such as ethyl alcohol, propyl alcohol or butyl alcohol. One particularly preferred monohydric aliphatic alcohol is ethanol. In some embodiments, the monohydric aliphatic alcohol is one having a solubility in water of 5 percent or greater. Methanol, ethanol, 1- and 2-propanol, and t-butyl alcohol, for example, are miscible with water, while 1-butanol has a solubility of about 9% in water and 2-butanol has a solubility in water of 7.7%. Preferably, the compositions as described herein advantageously comprise from 40% to 99%, (w/w), 40% to 95% (w/w), 50% to 99% by weight (w/w) monohydric aliphatic alcohol, or more preferably 60% to 85% (w/w) monohydric aliphatic alcohol. Representative ranges for the alcohol component, which may be combined with w/w amounts or ranges for other formulation components as provided herein are from: about 40-50%, 50-55% w/w, 50-60% w/w, 50-65% w/w, 50-70% w/w, 50-75% w/w, 50-80% w/w, 50-85% w/w, 50-90% w/w, 50-55% w/w, 55-60% w/w, 55-65% w/w, 55-70% w/w, 55-75% w/w, 55-80% w/w, 55-85% w/w, 55-90% w/w, 55-95% w/w, 60-65% w/w, 60-70% w/w, 60-75% w/w, 60-80% w/w, 60-85% w/w; 60-90% w/w, 60-95% w/w, 65-70% w/w, 65-75% w/w, 65-80% w/w, 65-85% w/w; 65-90% w/w, 65-95% w/w, 70-75% w/w, 70-80% w/w, 70-85% w/w, 70-90% w/w, 70-95% w/w, 75-80% w/w, 75-85% w/w, 75-90% w/w, 75-95% w/w, 80-85% w/w, 80-95% w/w, 80-95% w/w, 85-90% w/w, 85-95% w/w, 90-95% w/w, and 95-99% w/w.

Yet a further component of the topical composition (i.e., forming part of its solvent system) may be a polyol containing two or more hydroxyl groups, and possessing from 3-8 carbon atoms. Typically, the polyol is an aliphatic compound; polyols for use in the instant composition include diols such as propylene glycol (PG, propane-1,2-diol), hexylene glycol (2-methylpentane-2,4-diol), 1,3-butylene glycol (1,3-butane diol), and dipropylene glycol, triols such as glycerol and trimethylolpropane, and higher alcohols (meaning containing more than 3 hydroxyl groups) such as sorbitol and pentaerythritol. Preferred polyols are C3-C8 diols and triols. The diol or triol will typically have a molecular weight less than about 250, or even less than about 200. In some instances, the polyol will have a molecular weight less than about 125. The polyol, may, in some instances, be hygroscopic, such as in the case of propylene glycol. In some embodiments, the polyol is a triol other than glycerol or glycerin.

Tetracycline-class drugs are degraded by the presence of water but can be stabilized as described above such that they maintain stability for small amounts of water. This allows the use of solvents that are hygroscopic or aqueous, preferably in small amounts. In some embodiments, the solvent system for the topical composition comprises a monohydric aliphatic alcohol or a mixture of about 50% to 99% (w/w) of a monohydric aliphatic alcohol, such as ethanol, isopropanol, or tert-butyl alcohol. In some embodiments, the monohydric aliphatic alcohol is anhydrous. Some embodiments further comprise about 5% to about 30% (w/w) propylene glycol (CAS 57-55-6, Spectrum Chemical Manufacturing Co., New Brunswick, N.J.). The monohydric aliphatic alcohol is preferably ethanol, and more preferably anhydrous ethanol. In preferred embodiments, the concentration of propylene glycol is high enough that the tetracycline-class drug and the selective retinoid can remain in solution even after a volatile alcohol has penetrated into the skin or evaporated from it and low enough such that the tetracycline class drug is stable.

Topical compositions that achieve delivery of a drug across the stratum corneum and retention of a majority of the drug intracutaneously such that it does not enter the bloodstream in significant amounts are challenging to design and require innovative approaches. Several factors determine the permeability of the skin or of particular layers of the skin to a topically-applied drug. These factors include the characteristics of the skin, the characteristics of the drug (e.g., its size (molecular weight or molecular volume), lipophilicity/hydrophilicity, polarity, etc.), the dosage of drug applied, the concentration and volume of the composition to be applied, interactions between the drug and the delivery vehicle, interactions between the drug and the skin, and interactions of the drug and the skin in the presence of the ingredients in the delivery vehicle. Because of the multitude of factors involved in the topical administration of a drug, it is generally accepted that whether intracutaneous delivery of a drug can be successfully achieved is uncertain. Thus, topical administration, while desired from a patient convenience and drug delivery view, has been largely unsuccessful for many compounds as evidenced by the relatively few drugs approved for topical administration.

Topical compositions may desirably include more than two solvent components to draw on beneficial characteristics of three or more solvent components. Enhanced tazarotene solubility and minocycline solubility was observed in tertiary mixtures, as in binary mixtures, relative to the individual carboxylate ester solvent component (see, e.g., FIG. 2; Tables 5, 19 and 20) for mixtures comprising a carboxylate ester and ethanol. The addition of propylene or glycerol to the ethanol and carboxylate ester did not significantly affect the final solubility of tazarotene for the tested concentrations.

The amount of ethanol in formulations is desirably about 10-95% (w/w), more desirably about 30%-95% (w/w), 50%-95% (w/w), or 60%-90% (w/w). The amount of carboxylate ester is desirably about 5-95% (w/w), more desirably about 5-50% (w/w), 10-40% (w/w), or 20-40% (w/w). Such levels have several benefits, such as enhancing solubility of tazarotene and/or minocycline (as representative drugs) in a solvent mixture that has good organoleptic properties. Anhydrous ethanol is preferably used if the formulation includes a drug that is sensitive to water degradation.

Examples of solvents that could be beneficially used in a topical composition are propylene glycol (PG), isopropyl myristate (IM), di-isopropyl adipate (DP), and medium chain triglycerides (MCT). Propylene glycol is an organic compound that is used in cosmetics and beauty products because it helps as a humectant, a penetration enhancer, and a good solvent for many drugs. Isopropyl myristate and di-isopropyl adipate are synthetic oils used as emollient, skin conditioning agents, solvents, thickening agents, and penetration enhancers. Medium chain triglycerides are composed of a glycerol backbone and three fatty acids, wherein 2 or 3 of the fatty acid chains attached to glycerol groups are medium-chain in length (i.e., the fatty acids have an aliphatic tail of 6-12 carbon atoms). MCTs are commonly used as emollients and serve as excellent choices for compositions intended to be used on sensitive skin because they are lightweight and do not irritate most skin types.

Applicant has discovered that the penetration of tazarotene and of minocycline into human skin is enhanced by essentially completely solubilizing the drug in a mixture comprising a monohydric alcohol and a carboxylate ester with a ratio of ester groups to carbon atoms of at least 0.05. Penetration is further enhanced if the ratio of ester groups to carbon atoms for the carboxylate ester is larger, such as at least 0.05, 0.10, 0.15, 0.20, 0.25, 0.30, or 0.35. Exemplary carboxylate esters are shown in Table 1. It will be appreciated that other carboxylate esters having a ratio of ester groups to carbon atoms as known in the art are suitable for use in the compositions described herein. This enhanced penetration permits better bioavailability of tazarotene and of minocycline (as representative drugs) and beneficially reduces the residual drug on the surface of the skin after treatment. This enhanced penetration has multiple benefits: First, reducing the amount of residual drug on the skin surface can reduce the intensity and incidence rate of side effects such as itchiness, dry and cracking skin, redness, and photosensitivity. Second, the enhanced bioavailability of drug reduces the amount of drug that is required to achieve an effective concentration within the skin. This reduces the side effect profile further by allowing a lower concentration of drug(s) to be used within the composition.

TABLE 1
EXEMPLARY CARBOXYLATE ESTERS

| | | Molecular | |
|---|---|---|---|
| Name | Chemical formula | weight | Chemical structure |
| Isopropyl myristate | C₁₇H₃₄O₂ | 270.46 | 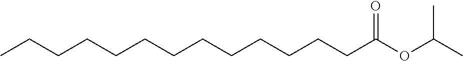 [SMILES: CCCCCCCCCCCCCC(=O)OC(C)C] |
| Kollisolv MCT 70 | triglycerides whose fatty acids have an aliphatic tail of 6-12 carbon atoms. | varies | 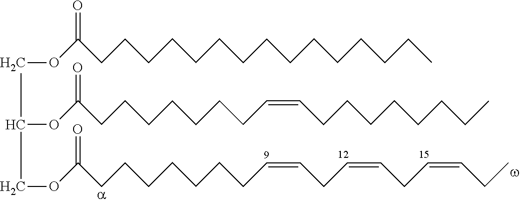 [SMILES: CC/C=C\C/C=C\C/C=C\CCCCCCCC(=O)OCC(COC(=O)CCCCCCCCCCCCCCC)OC(=O)CCCCCCC/C=C\CCCCCCCC] |
| Diisopropyl Adipate | C₁₂H₂₂O₄ | 230.3 | 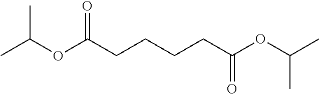 [SMILES: CC(C)OC(=O)CCCCC(=O)OC(C)C] |
| Ethyl acetate | C₄H₈O₂ | 88.11 | 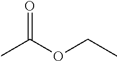 |
| Triacetin | C₉H₁₄O₆ | 218.21 | 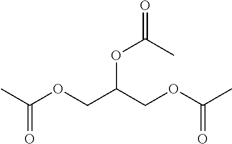 |
| Dimethyl Succinate | C₆H₁₀O₄ | 146.14 | 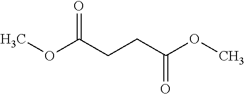 |
| Propyl Acetate | C₅H₁₀O₂ | 102.13 | 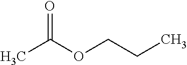 |
| Amyl Acetate | C₇H₁₄O₂ | 130.19 | 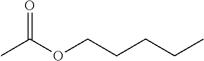 |
| Prenyl Acetate | C₇H₁₂O₂ | 128.17 | 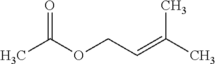 |
| Benzyl Acetate | C₉H₁₀O₂ | 150.18 | 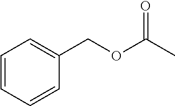 |
| Benzyl Propionate | C₁₀H₁₂O₂ | 164.2 | 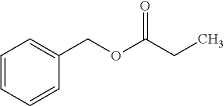 |

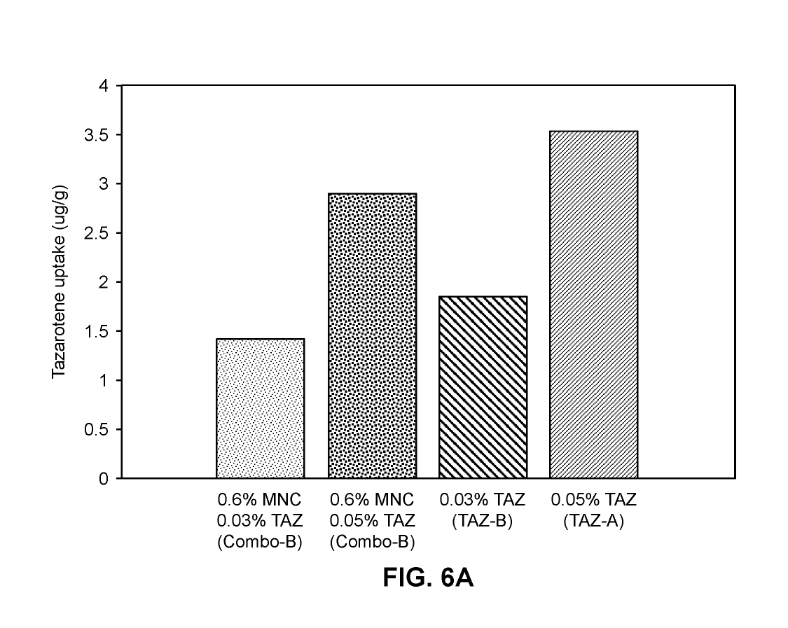

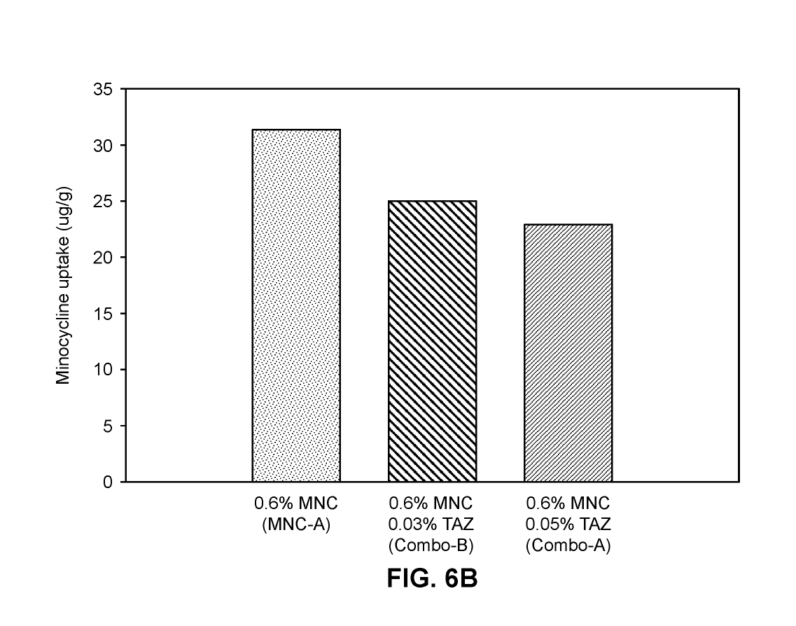

As described in Example 6 and shown in FIGS. 6A-6B, combining a selective retinoid and a tetracycline class antibiotic in compositions as described herein does not inhibit the topical penetration of either the selective retinoid or the tetracycline class antibiotic.

Adapalene dissolves particularly well in cineole, which is miscible with isopropanol, ethanol, propylene glycol, and many other pharmaceutically acceptable solvents. Cineole can be added to the monohydric aliphatic alcohol or to the mixture of a monohydric aliphatic alcohol and propylene glycol described above.

While alcohol-based compositions can provide high levels of stability for both a tetracycline-class drug and a selective retinoid, the alcohol in such compositions can have a stinging sensation when applied, especially to skin that has been irritated or inflamed by a dermatological condition or disease. Additionally, selective retinoids may irritate and/or dry out the skin, which can be uncomfortable for the patient, particularly during periods of accelerated skin cell turnover. For these reasons, some embodiments beneficially do not contain a monohydric aliphatic alcohol. Other embodiments do not contain more than about 10% monohydric aliphatic alcohol, more than about 25% monohydric aliphatic alcohol, or more than about 50% monohydric aliphatic alcohol.

Adapalene and tazarotene are known to be strongly lipophilic molecules with log P values of approximately 6 and 4.3, respectively. In their salt forms, tetracycline-class drugs are typically poorly solubilized in highly lipophilic solvent systems. Mixtures can be formed in which the tetracycline-class drug is suspended in a lipophilic solvent system, such as white petrolatum. However, such compositions are not preferred because such suspensions typically reduce the penetration of the tetracycline-class drug into the skin and may increase the variability of the concentration of the tetracycline-class drug from one portion of the topical composition to another. The solvent systems described herein have the benefit where both tetracycline-class drugs and selective retinoids are suitably dissolved. For example, adapalene, tazarotene, and minocycline each dissolve in ethanol. However, such solutions may have other undesirable characteristics, as noted above.

In some embodiments, the compositions described herein have a limited alcohol content in order to reduce these undesirable characteristics. One method that can limit the alcohol content in the topical composition is to use an emulsion with a tetracycline-class drug (e.g., minocycline or doxycycline) in a hydrophilic phase (e.g., ethanol) and a lipophilic selective retinoid (e.g., adapalene or tazarotene) in a lipophilic phase (e.g., a fluorinated oil). Further examples of emulsions that could be useful for such compositions are given in U.S. Pat. No. 9,474,720. Examples of other suitable emulsions will be evident to those skilled in the art. Emulsions can be, for example, oil-in-water type emulsions, water-in-oil type emulsions, or more complicated three or four level emulsions, such as oil-in-water-in-oil type emulsions. In many preferred embodiments, the hydrophilic phase of an emulsion does not contain any water, despite the terminology, such as “oil-in-water.” Similarly, the hydrophobic phase does not require the inclusion of oil. In some preferred embodiments, the “water” phase has a water content of less than 5% when measured by Karl Fischer titration.

A topical composition containing a selective retinoid (e.g., tazarotene or adapalene) and a tetracycline-class drug (e.g., minocycline) can have many beneficial characteristics: strong antibiotic activity, low incidence of bacterial resistance, anti-inflammatory properties, inhibits keratinocyte proliferation, and/or reduces comedo count. A topical composition that contains a selective retinoid and a tetracycline-class drug can address some or all of the four factors of acne simultaneously: excess sebum production, ductal hypercornification, proliferation of P. acnes, and increased inflammation.

In some preferred embodiments, the topical composition enables effective transfollicular delivery of minocycline and contains tazarotene or adapalene. The combined use of minocycline and either tazarotene or adapalene enables use of lower doses of each, which mitigates the incidence rate and/or the severity of photosensitivity for minocycline and mitigates the incidence rate and/or of the severity of post-inflammatory hyperpigmentation. Each of adapalene and tazarotene also modulates RAR-beta synergistically with minocycline, which accumulates in fibroblasts, to reduce post inflammatory acne scarring.

There are significant benefits to being able to combine the tetracycline-class drug and the selective retinoid into a single topical composition. Such benefits include, for example, ease of use, improved patient compliance due to only requiring a single topical application rather than multiple applications, and dosage uniformity across a treatment area due to the ability to use a homogenous distribution of the active ingredients.

Alternatively, there are also significant benefits if the tetracycline-class drug and the selective retinoid are prepared in separate compositions and combined just prior to use or at the time of use. Such benefits include the ability to develop optimal compositions for each of the two drugs such that stability is enhanced. This is particularly important for the tetracycline-class drug, since such drugs can be undesirably unstable in many topical compositions. Two or more of such compositions may be sold together in a kit that comprises 1) a first topical composition comprising a tetracycline-class drug and 2) a second topical composition that comprises a selective retinoid.

Despite the benefits of combining the tetracycline-class drug and the selective retinoid into a single topical composition, there are significant challenges to making such compositions stable due to the rapid epimerization of tetracycline-class drugs.

In preferred embodiments, including many of the exemplary embodiments listed in the Examples, the concentration of the epimer of the tetracycline-class drug is less than about 15% (w/w) or about 10% (w/w) of the concentration of the active drug when stored for at least 3 months at 40° C., optionally in a dark environment in a sealed glass container. In preferred embodiments, including many of the exemplary embodiments listed in the Examples, the tetracycline-class drug (e.g. minocycline or doxycycline) is stable when stored for at least 3 months at 40° C. to 25° C., optionally in a dark environment in a sealed glass container. In preferred embodiments, including many of the exemplary embodiments listed in the Examples, the selected retinoid (e.g. tazarotene or adapalene) is stable when stored for at least 6 and/or 12 months at 20-25° C., optionally in a dark environment in a sealed glass container. In preferred embodiments, including the exemplary embodiments listed in the Examples, compositions comprising a tetracycline-class drug and/or a selective retinoid can be produced to exhibit improved stability when compared to compositions containing a tetracycline-class drug or selective retinoid as the sole active ingredient when stored for at least 6 and/or 12 months at 20° C. to 25° C. in a dark environment in a sealed glass container.

Epimerization of tetracycline-class drugs is a common problem for creating stable topical compositions when combined with certain materials, for example, many aqueous solvents. In some embodiments, the tetracycline-class drug can be provided in an equilibrium state with its epimer such that stability can be achieved in the presence of reactive agents. In some such embodiments, the ratio of the epimer of the tetracycline-class drug to the tetracycline-class drug by weight is in the range of about 1:10 to 1:3.

For treatment of some dermatological conditions and diseases, such as acne, rosacea, psoriasis, hidradenitis suppurativa, Hailey-Hailey, Darier's disease, keratosis pilaris, and actinic keratosis, retinoic acids and retinoids that are not selective, such as tretinoin, isotretinoin, or alitretinoin, may be used in place of the selective retinoid in combination with a tetracycline-class drug, such as minocycline, doxycycline, or tetracycline. Such combinations will have at least some of the benefits described herein for the combination of selective retinoids and a tetracycline-class drug and will have other benefits not described herein. In most cases, such combinations are not the preferred choice of dosage form due to the advantages listed herein for the combination of a tetracycline-class drug with a selective retinoid. However, there will be dermatological conditions for which such combinations of a tetracycline-class drug with a retinoic acid or a nonselective retinoid are preferred.

In one embodiment, the topical composition does not bleach a cloth when placed in contact with the cloth for about one hour in a dark environment at 20° C. to 25° C. and 60% relative humidity.

The topical composition comprising a combination of a selective retinoid, such as adapalene or tazarotene, and a tetracycline-class drug, such as minocycline or doxycycline, can be used for treatment or prophylaxis of a dermatological condition or disease. Patients typically apply the topical composition daily for a period of 6 to 52 weeks, or a period for at least 12 weeks. Shorter treatment periods are also possible for certain conditions. The topically applied composition can deliver the selective retinoid and the tetracycline-class drug in separate stable formulations or in a single combination composition.

Compositions comprising a tetracycline-class drug and a selective retinoid can also have a reduced rate of development of drug resistant strains of P. acnes in comparison to compositions containing tetracycline-class drugs as the sole active ingredient.

The instant compositions may also contain relatively small amounts, e.g., less than about 10% (w/w) of one or more auxiliary excipients suitable for topical use including but not limited to pH modifying agents, preservatives, thickening agents, gel-forming agents, emulsifying agents, antioxidants, scent agents, and the like. Compounds suitable for incorporation may be found, e.g., in R. C. Rowe, et al., Handbook of Pharmaceutical Excipients (4th Ed.), Pharmaceutical Press, London, 2003.

In some embodiments, the topical compositions comprise one or more gelling agents. Gelling agents which may be used in the topical compositions include conventional gelling agents well known for their gelling properties, such as, for example, cellulose ethers such as hydroxypropyl cellulose, hydroxypropyl methylcellulose, carboxymethyl cellulose, sodium carboxymethyl cellulose, hydroxyethyl cellulose, and the like; vinyl alcohols; vinyl pyrrolidones; natural gums such as karaya gum, locust bean gum, guar gum, gelan gum, xanthan gum, gum arabic, tragacanth gum, carrageenan, pectin, agar, alginic acid, sodium alginate and the like, and methacrylates such as those available under the tradename Eudragit® from Rohm Pharma. Other gelling agents include polyoxyethylene-polyoxypropylene copolymers (poloxamers) such as those available under the tradename “Lutrol®”, and the like. Preferred gelling agents are those absent free carboxyl groups such as, for instance, hydroxypropylcellulose, hydroxypropylmethylcellulose, hydroxyethylcellulose, methylcellulose, organo/cold water soluble cellulose, hydroxyethylmethylcellulose, ethylcellulose, ethyl(hydroxyethyl)cellulose. For substituted celluloses, a moderate to high degree of substitution is preferred in order to limit the impact of hydroxyl groups on the stability of the tetracycline drug and/or in order to increase the solubility of the gelling agent in a selected solvent system. The preferred degree of substitution is at least 1.0, or preferably in the range of 1.2 to 6.0, or more preferably in the range of 2.5 to 4.5.

The composition may also contain at least one antioxidant. The amount of antioxidant, if present, will typically range from about 0.005% to about 15.0% by weight of the composition. Illustrative ranges include from about 0.005 to about 3.0 wt %, 0.01% to about 2.5% by weight antioxidant, from about 0.05% to about 2% by weight antioxidant, and from about 0.1% to about 1.5% by weight anti-oxidant. Illustrative amounts of antioxidant include 0.01%, 0.025%, 0.05%, 0.075%, 0.1%, 0.2%, 0.3%, 0.4%, 0.5%, 0.6%, 0.7%, 0.8%, 0.9% and 1% by weight. In one embodiment, the amount of antioxidant comprised within the composition is 0.01% by weight. In another embodiment, the amount of antioxidant comprised within the formulation is 0.2% by weight. Suitable antioxidants include, for example, butylated hydroxyanisole (BHA), butylated hydroxytoluene (BHT), tertiary butyl hydroquinone, propyl gallate, α-tocopherol, sodium metabisulfite, and the like. One preferred class of antioxidants are sulfur-containing antioxidants such as sodium metabisulfite, glutathione, N-acetylcysteine, thioproline, and taurine. Additional preferred compositions comprise at least one antioxidant selected from the list consisting of a sulfite compound, BHT, sodium selenite, DL-alpha tocopherol, a combination of dithioerythreitol and DL-alpha tocopherol, and sodium erythorbate. Sulfurous acid salts, thiosufuric acid salts, and organic esters (referred to collectively as “sulfites”) are also preferred, such as bisulfites, pyrosulfites, metabisulfites, and sulfites.

In one or more embodiments, the topical composition comprises a suitable amount (e.g., about 0.005% to about 15% by weight, or between about 0.005-10 wt %, 0.005-7.5 wt % or 0.005-3.0 wt %) of a sulfite compound and/or a thiosulfate compound, e.g., a sulfite, a metabisulfite, a bisulfite salt or a thiosulfate salt, where the sulfite is accompanied by a suitable counterion. Sulfite and thiosulfate antioxidants are particularly advantageous because they inhibit 4-epi-minocycline formation in and discoloration of topical minocycline compositions. Organic sulfite/thiosulfate compounds may also be employed, such as organic esters of sulfurous acid, acyclic sulfites, and cyclic sulfites. Exemplary organic sulfites include ethyl, p-tolyl and isopropyl sulfites, although any suitable organic sulfite may be employed.

The composition may further contain one or more preservatives in an amount typically ranging from about 0.01% to about 2.0% by weight of the composition. Illustrative preservatives include, for example, phenoxyethanol, methyl paraben, propyl paraben, butyl paraben, benzyl alcohol, and the like.

The topical composition may also comprise a small amount, such as about 0.1% to 10% by weight, of one or more compounds effective to introduce a favorable scent or aroma, such as a natural oil or other suitable agent. Suitable essential oils include, for example, plant essential oils from eucalyptus, frankincense, patchouli, peppermint, lemon, lavender, orange, rosehip, rosemary, tea tree, jasmine, and the like. For example, in one or more embodiments, the composition comprises a small amount, such as about 0.1% to 5% by weight, of 1,8-cineole, or some other essential oil.

It has been discovered that the use of certain carboxylate esters (e.g., dimethyl succinate, propyl acetate, or combinations thereof) can be particularly effective for desirable usability characteristics, such as good smell, of the topical compositions without compromising penetration, solubility, and/or stability of the compositions.

The combination of polyol and 1,8-cineole can be particularly effective in preventing the skin from scaling and extreme dryness, especially when administration is for an extended period of time, e.g., for about 2 weeks or more. Signs of dry skin which can be prevented include both scaling and itching.

The topical composition may be in a number of different forms, including, for example, a solution, liquid, spray, foam, lotion, gel and the like. Preferably, the composition is a liquid, has good stability, adheres to the skin, and has a smooth feel. Preferably, the composition is not an emulsion. Generally, preferred compositions are absent nanoparticles and/or microparticles, although in some instances, the composition may comprise nanoparticles and/or microparticles. For additional information regarding suitable formulations, see, for example, “Remington: The Science and Practice of Pharmacology,” 22nd edition, (Pharmaceutical Press, 2013).

The composition may be prepared by, e.g., admixture of the ingredients typically through the use of vigorous agitation such as high shear mixing. Mixing can also be accomplished by any suitable method using any suitable manual or automated means. Optional additional steps include those which result in the addition of one or more of the optional auxiliary ingredients as set forth above. Methods for preparing a pharmaceutical formulation are well known in the art and are described, for example, in Handbook of Pharmaceutical Formulations: Liquid Products, Vol 3, S. Niazi., CRC Press, 2004.

The composition may be topically applied directly to the affected areas of the skin, for example, using the fingertips, a sponge applicator, a cotton applicator, by spraying, aerosolization, or any other suitable method. The compositions provided herein are useful for treating any condition that is susceptible to treatment with a tetracycline class drug such as minocycline and/or for treating a condition that is susceptible to treatment with a selective retinoid such as tazarotene or adapalene. The compositions provided herein may be used, for example, for treating conditions such as acne, impetigo, hidradenitis suppurativa, Hailey-Hailey, Darier's disease, cellulitis, erysipelas, folliculitis, furuncles, carbuncles, Lyme disease and other skin infections, rosacea, seborrheic dermatitis, bullous dermatoses, cutaneous sarcoidosis, Kaposi's sarcoma, and neutrophilic dermatoses, and inflammation associated therewith. Types of acne include, for example, acne vulgaris, acne rosacea, acne inversa, acne conglobata, acne fulminans, gram-negative folliculitis, and pyoderma faciale, among others. For example, the composition may be used for treating moderate to severe acne, and the acne may be nodular or cystic.

In one or more embodiments, the method comprises the step of administering a topical composition as provided herein to an accessible body surface of a human or an animal in need of such treatment. Generally, the composition is applied in a conventional amount from once to several times weekly or daily on the affected areas of the skin, until the acne or condition being treated has visibly diminished or disappeared. For example, the topical composition may be applied topically at least once daily for a period of at least 1 month, or may be applied to the skin once or twice daily for a period of from 6 to 52 weeks or even longer. The number of applications and course of treatment will vary with the severity of the condition being treated, patient considerations, and the like. Thus, the composition may, in certain instances by applied one daily, twice daily, once every other day, from one to three times weekly, from 1 to 4 times weekly, every 3 days, etc.

A conventional amount is an amount that is sufficient to spread, e.g., thinly spread, over the affected area. If desired, the efficacy of treatment may be quantified by using a grading system such as the Leeds system (O' Brien, S C., et al., J. Dermatol Treat 1998; 9:215-220), the Comprehensive Acne Severity Scale (Tan, J K, et al., J. Cutan Med Surg 2007 November; 11(6):211-6), or the Global Acne Grading System (Doshi, A., et al., Int. J. Dermatol 1997 June 36(6); 416-8). In one or more embodiments, the efficacy of treatment is assessed by a visual examination of the affected area. In some cases, prophylactic treatment may be continued even if the condition has visibly diminished or disappeared, as a preventative measure. In some embodiments, the efficacy of treatment is assessed by an evaluation of a reduction in total lesion count, where application of a topical composition as described herein is effective to result in a reduction in total lesion count as measured from the commencement of treatment.

Example 1 presents data from ex vivo drug penetration studies on human tissue samples. These experiments quantify the amount of minocycline and tazarotene that penetrates beyond the first couple layers of the skin following topical application of the composition and demonstrate (i) that the efficiency of penetration increases as the ratio of ester groups to carbon atoms of the carboxylate ester increases and (ii) good efficiency of penetration into the skin.

Example 2 demonstrates that the solubility of tazarotene can be enhanced by mixtures of ethanol and a carboxylate ester relative to either ethanol or the carboxylate ester individually. Such solubility enhancement can also be seen in mixtures of three or more components if two of them are ethanol and a carboxylate ester.

Example 3A demonstrates that a selective retinoid and a tetracycline-class antibiotic can be stable when combined into a single composition in which both drugs are dissolved. Exemplary embodiments were tested with tazarotene and minocycline in combination and individually.

Example 3B demonstrates that a tetracycline-class drug can be stable when combined in a single composition that comprises an antioxidant and a divalent cation.

Example 4 presents ex vivo penetration data for tazarotene in exemplary embodiments in comparison to a commercially available topical composition comprising tazarotene (TAZORAC).

Example 5 presents exemplary compositions that demonstrate that topical compositions that comprise both a selective retinoid and a tetracycline-class antibiotic can be produced in which both drugs are stable and where the drugs in combination exhibit better stability than the drugs individually.

Example 6 presents exemplary compositions comprising a selective retinoid and a tetracycline-class antibiotic. These compositions show that the topical uptake of each of the drugs is not inhibited by the presence of the other and that each of the drugs can penetrate into human skin.

Example 7 presents ex vivo penetration data for minocycline from exemplary compositions.

Example 8 provides additional exemplary embodiments that comprise both a selective retinoid and a tetracycline-class antibiotic.

Example 9 presents exemplary compositions demonstrating that topical compositions that comprise both a selective retinoid and a tetracycline-class antibiotic do not inhibit the efficiency of topical penetration of either active pharmaceutical agent in human skin.

Example 10 presents exemplary compositions demonstrating that topical compositions that comprise both a selective retinoid and a tetracycline-class antibiotic can be produced in which both drugs are stable and where the drugs in combination exhibit better stability than the drugs individually.

Example 11 presents data from an animal study of a P. acnes bacteria triggered inflammatory model in which P. acnes bacteria were injected into the skin near the shoulders and upper back of SKH-1 hairless mice. Compositions comprising tazarotene and minocycline were topically applied and compared to topical controls. The effectiveness of reduction in acne was evaluated by measuring the lesion volume after 14 days.

Example 12 presents exemplary compositions comprising adapalene and/or minocycline. A stability study was performed in which these samples were stored in sealed amber glass containers for a period of 8 weeks at 40° C. The results demonstrate that compositions that comprise both adapalene and minocycline can be produced such that both drugs are stable.

Example 13 demonstrates that the solubility of antioxidants can be enhanced by the presence of a tetracycline-class drug.

Examples 14-18 present exemplary compositions demonstrating that topical compositions that comprise a tetracycline-class antibiotic can be produced such that the drug is fully soluble and stable. In particular, Example 16 presents exemplary compositions demonstrating a tetracycline-class drug can be stable when comprises a divalent cation and an antioxidant as sulfite and thiosulfate. Example 17 demonstrates that a tetracycline-class drug at difference strengths can be stable when combined in a single composition that comprises an antioxidant or antioxidant combination and a divalent cation. Example 18 presents exemplary compositions demonstrating a tetracycline-class drug can be stable when comprises a compound providing a divalent cation and an antioxidant compound, or a single compound providing both divalent cation and antioxidant anion.

Examples 19-20 present in vivo data from compositions described herein.

### EXAMPLES

The following examples are put forth to provide those of ordinary skill in the art with a complete disclosure and description of how the composition, its components, active ingredients, solvents, and the like, are prepared and evaluated, along with related methods, and are intended to be purely exemplary. Thus, the examples are in no way intended to limit the scope of what the inventors regard as their invention. There are numerous variations and combinations, e.g., component concentrations, desired solvents, solvent mixtures, antioxidants, and other mixture parameters and conditions that may be employed to optimize composition characteristics such as purity, yield, stability, odor, color, viscosity, penetration, and the like. Such are considered as well within the scope of the present disclosure.

Unless otherwise indicated, the following examples were conducted at about room temperature, e.g. about 20-25° C. or 25° C. specifically, and at atmospheric pressure.

In each of the following examples, the form of minocycline hydrochloride that was used was minocycline hydrochloride dihydrate, which is shortened in the description of the examples to minocycline hydrochloride. It will be evident to those skilled in the art how compositions can be made using other salts and hydrates of minocycline.

### Example 1

### Tazarotene and Minocycline Penetration into Ex Vivo Human Skin

Penetration experiments with ex vivo human skin tissue were conducted to determine whether minocycline and tazarotene penetrate into the skin in sufficient concentrations to achieve a desired therapeutic effect when comprised within compositions that are applied to the skin surface and the composition comprises a monohydric aliphatic alcohol, a polyol, a carboxylate ester, a magnesium salt, and a sulfite. The penetration into facial skin was assessed for three different human donors with two samples from each donor for each data point.

Solvent mixtures were prepared in the proportions described in Table 2, with each solvent mixture comprising anhydrous ethanol (Spectrum Chemicals, Gardena, Calif.), propylene glycol (Spectrum Chemicals, Gardena, Calif.) or glycerol (Spectrum Chemicals, Gardena, Calif.), and a selected carboxylate ester. To each solvent mixture was added 1.2% (w/w) minocycline hydrochloride (Euticals S.P.A, Origgio, Italy) (1.0% base equivalent), 0.05% (w/w) tazarotene (AvaChem Scientific, San Antonio, Tex.), 1.2% (w/w) magnesium chloride (anhydrous) (Sigma-Aldrich Corp., St. Louis, Mo.), 0.20% sodium metabisulfite (Spectrum Chemicals, Gardena, Calif.), and 0.60% (w/w) hydroxypropyl cellulose HF (KLUCEL HF, Ashland, Inc., Covington, Ky.).

TABLE 2
Compositions for Skin Penetration Study with Varied Solvent Systems

| COMPOSITION | | | | |
|---|---|---|---|---|
| COMPONENT | C | C.1 | C.2 | C.3 |
| minocycline | 1.20% | 1.20% | 1.20% | 1.20% |
| hydrochloride | | | | |
| tazarotene | 0.05% | 0.05% | 0.05% | 0.05% |
| hydroxypropyl | 0.60% | 0.60% | 0.60% | 0.60% |
| cellulose HF | | | | |
| magnesium chloride, | 1.20% | 1.20% | 1.20% | 1.20% |
| anhydrous | | | | |
| ethanol, anhydrous | 75.75% | 65.75% | 65.75% | 65.75% |
| propylene glycol | 20.00% | 20.00% | 20.00% | 20.00% |
| cineole | 1.00% | 1.00% | 1.00% | 1.00% |
| Kollisolv MCT 70 | — | 10.00% | — | — |
| isopropyl myristate | — | — | 10.00% | — |
| diisopropyl adipate | — | — | — | 10.00% |
| sodium metabisulfite | 0.20% | 0.20% | 0.20% | 0.20% |

| COMPOSITION | | | | | |
|---|---|---|---|---|---|
| COMPONENT | C.1.1 | C.3.1 | C.2.3 | C.6.1 | C.6.2 |
| minocycline | 1.20% | 1.20% | 1.20% | 1.20% | 1.20% |
| hydrochloride | | | | | |
| tazarotene | 0.05% | 0.05% | 0.05% | 0.05% | 0.05% |
| hydroxypropyl | 0.60% | 0.60% | 0.60% | 0.60% | 0.60% |
| cellulose HF | | | | | |
| magnesium chloride, | 1.20% | 1.20% | 1.20% | 1.20% | 1.20% |
| anhydrous | | | | | |
| ethanol, anhydrous | 75.75% | 75.75% | 60.00% | 75.75% | 60.00% |
| propylene glycol | 10.00% | 10.00% | 15.75% | | |
| glycerol | — | — | — | 10.00% | 15.75% |
| cineole | 1.00% | 1.00% | 1.00% | 1.00% | 1.00% |
| Kollisolv MCT 70 | 10.00% | — | — | — | — |
| isopropyl myristate | — | — | 20.00% | 10.00% | 20.00% |
| diisopropyl adipate | — | 10.00% | — | — | — |
| sodium metabisulfite | 0.20% | 0.20% | 0.20% | 0.20% | 0.20% |

| COMPOSITION | | | | |
|---|---|---|---|---|
| COMPONENT | C.7.2 | C.9.1 | C.11.1 | C.12.1 |
| minocycline | 1.20% | 1.20% | 1.20% | 1.20% |
| hydrochloride | | | | |
| tazarotene | 0.05% | 0.05% | 0.05% | 0.05% |
| hydroxypropyl | 0.60% | 0.60% | 0.60% | 0.60% |
| cellulose HF | | | | |
| magnesium chloride, | 1.20% | 1.20% | 1.20% | 1.20% |
| anhydrous | | | | |
| ethanol, anhydrous | 60.00% | 74.18% | 67.85% | 60.00% |
| propylene glycol | 15.75% | 19.57% | 17.90% | |
| glycerol | — | — | — | 15.75% |
| cineole | 1.00% | 1.00% | 1.00% | 1.00% |
| Ethyl Acetate | 20.00% | — | — | 20.00% |
| AZONE ® | — | 2.00% | — | — |
| Lauryl alcohol | — | — | 10.00% | — |
| (LIPOCOL L) | | | | |
| sodium metabisulfite | 0.20% | 0.20% | 0.20% | 0.20% |

The compositions were applied to skin samples from three human donors at a gel volume of 2.5 mg/cm². Tissue was maintained in a damp environment to limit drying of the tissue and incubated at 32° C. for 4 hours. At the end of the incubation period, excess composition was wiped from the surface using first a dry gauze pad, second a gauze pad soaked with 70% isopropyl alcohol, and finally with a dry gauze pad. One six (6) millimeter punch biopsy was taken from within each test area. From each of the biopsies, minocycline or tazarotene was extracted using acidified methanol. The supernatants were analyzed by high performance liquid chromatography.

The ratio of the number of ester groups to the number of carbon atoms in the carboxylate ester is presented in Table 3 for each tested composition.

TABLE 3
Ratio of Number of Ester Groups to Number of Carbon Atoms for the Carboxylate Ester
Solvent in the Exemplary Compositions

| | | | | RATIO OF | | |
|---|---|---|---|---|---|---|
| | | | | ESTER | | |
| | | | NUMBER | GROUPS | | |
| | | NUMBER | OF | TO | NORMALIZED | NORMALIZED |
| EXEMPLARY | CARBOXYLATE | OF ESTER | CARBON | CARBON | MINOCYCLINE | TAZAROTENE |
| COMPOSITION | ESTER | GROUPS | ATOMS | ATOMS | UPTAKE | UPTAKE |
| C.1 | Kollisolv MCT | 3 | ~30 | ~0.1 | 0.56 | 0.38 |
| | 70 | | | | | |
| C.2 | isopropyl | 1 | 17 | 0.059 | 0.57 | 0.35 |
| | myristate | | | | | |
| C.3 | diisopropyl | 2 | 12 | 0.167 | 0.81 | 0.38 |
| | adipate | | | | | |
| C.1.1 | Kollisolv MCT | 3 | ~30 | ~0.1 | 0.39 | 0.30 |
| | 70 | | | | | |
| C.3.1 | diisopropyl | 2 | 12 | 0.167 | 0.62 | 0.61 |
| | adipate | | | | | |
| C.2.3 | isopropyl | 1 | 17 | 0.059 | 0.43 | 0.33 |
| | myristate | | | | | |
| C.6.1 | isopropyl | 1 | 17 | 0.059 | 0.46 | 0.46 |
| | myristate | | | | | |
| C.6.2 | isopropyl | 1 | 17 | 0.059 | 0.45 | 0.31 |
| | myristate | | | | | |
| C.7.2 | ethyl acetate | 1 | 4 | 0.25 | 1.03 | 1.19 |
| C.11.1 | lauryl alcohol | NA | 12 | NA | 0.68 | 0.60 |
| | (LIPOCOL L) | | | | | |
| C.12.1 | ethyl acetate | 1 | 4 | 0.25 | 0.79 | 0.65 |

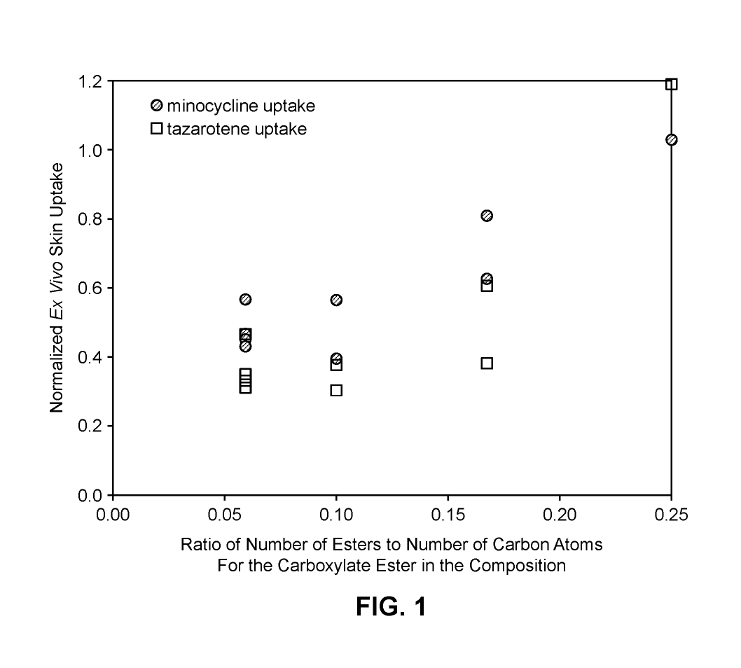

The average values of the six donor samples for each concentration are presented in FIG. 1. The results demonstrate that the efficiency of penetration of both minocycline and tazarotene increases as the ratio of ester groups to carbon atoms in the carboxylate ester increases. Good efficiency of penetration into the skin is also demonstrated for both tazarotene and minocycline.

### Example 2

### Solubility of Tazarotene

Tazarotene is known to have low solubility in many traditional solvent systems. The solubility of tazarotene in ethanol is relatively good at about 21 mg/g. However, compositions with ethanol as the only solvent are typically irritating to the skin, have a pungent smell, and leave the skin feeling dry. Aspects of the solvent system can be desirably improved by adding other solvents while maintaining sufficient solubility of tazarotene. It has been discovered that that mixtures of ethanol and carboxylate esters have higher solubility of tazarotene than would be expected from mixtures of the components individually.

As shown in Table 4, carboxylate esters alone do not demonstrate good solubility for tazarotene. Tazarotene is desirably used in compositions at a concentration of 0.01% to 0.20% (w/w). So, solubilites of at least about 0.1 to 2.0 mg/g are required to solubilize tazarotene. However, significantly higher solubilities are generally desirable so that tazarotene does not precipitate out of the composition and will remain solubilized for a longer period after the composition is applied to the skin and the solvent evaporates or penetrates into the skin. For these reasons, tazarotene solubility of at least 10 mg/g is desired, preferably at least 20 mg/g, at least 30 mg/g, at least 40 mg/g, or at least 50 mg/g.

TABLE 4
Solubility of Tazarotene in Individual Solvent Components

| | TAZAROTENE |
|---|---|
| SOLVENT | SOLUBILITY |
| COMPONENTS | (mg/g) |
| ethanol | 21.2 |
| propylene glycol | 0.0054 |
| isopropyl myristate | 0.75 |
| diisopropyl adipate | 1.21 |
| medium chain triglycerides | 0.16 |
| dimethyl succinate | 63.18 |
| propyl acetate | 138.04 |

The solubility of tazarotene was significantly improved by selected binary and tertiary mixtures of ethanol and a carboxylate ester as shown in Table 5. Additionally, FIG. 2 shows the tazarotene solubility for binary mixtures of ethanol and other tested solvent components as the mixture ratio is varied. As shown in Table 5 and FIG. 2, the tazarotene solubility in selected binary and tertiary mixtures was even higher than the solubility of tazarotene in ethanol alone. Although many binary mixtures showed higher solubility than the individual components, the highest solubility was observed in mixtures of ethanol and carboxylate esters. Even small amounts of carboxylate esters can have unexpectedly large effects. For example, the solubility in the mixture of 90% ethanol and 10% isopropyl myristate is 34.8 mg/g compared to only 21.2 mg/g in ethanol alone and 0.75 mg/g in isopropyl myristate alone.

TABLE 5
Solubility of Tazarotene in Binary and
Tertiary Mixtures of Solvent Components

| | MIXTURE | TAZAROTENE |
|---|---|---|
| | RATIO | SOLUBILITY |
| SOLVENT COMPONENTS | (W/W) | (mg/g) |
| ethanol and isopropyl | 9:1 | 34.8 |
| myristate | | |
| ethanol and diisopropyl | 9:1 | 23.3 |
| adipate | | |
| ethanol and medium chain | 9:1 | 23.5 |
| triglyceride | | |
| ethanol and isopropyl | 1:1 | 37.8 |
| myristate | | |
| ethanol and diisopropyl | 1:1 | 39.0 |
| adipate | | |
| ethanol and medium chain | 1:1 | 38.6 |
| triglyceride | | |
| ethanol and propylene | 4:1 | 22.3 |
| glycol | | |
| ethanol and propylene | 1:1 | 10.3 |
| glycol | | |
| propylene glycol and | 2:1 | 6.0 |
| isopropyl myristate | | |
| propylene glycol and | 2:0 | 5.6 |
| diisopropyl adipate | | |
| propylene glycol and | 2:1 | 1.27 |
| dimethyl succinate | | |
| propylene glycol and | 2:1 | 1.55 |
| propyl acetate | | |
| ethanol, propylene glycol, | 7:2:1 | 32.3 |
| and isopropyl myristate | | |
| ethanol, propylene glycol, | 7:2:1 | 24.9 |
| and diisopropyl adipate | | |
| ethanol, propylene glycol, and | 7:2:1 | 24.4 |
| medium chain triglycerides | | |
| ethanol, propylene glycol, | 7:2:1 | 30.1 |
| and dimethyl succinate | | |
| ethanol, propylene glycol, | 7:2:1 | 29.1 |
| and propyl acetate | | |
| ethanol, glycerol, and | 7:2:1 | 30.3 |
| isopropyl myristate | | |

The combination of ethanol with carboxylate esters (e.g. isopropyl myristate, diisopropyl adipate, and medium chain triglycerides) resulted in unique mixtures which significantly improved the solubility of tazarotene. Thus, the mixtures comprising ethanol and a carboxylate ester were shown to form a solvent with beneficial and unexpected characteristics relative to the individual components. This increased solubility was not seen in mixtures of ethanol and propylene glycol or in mixtures of propylene glycol and a carboxylate ester (e.g. isopropyl myristate and diisopropyl adipate). Instead, these mixtures followed expected behavior for such binary mixtures of solvent components.

Table 6 shows exemplary combinations of ethanol with a carboxylate ester (e.g., dimethyl succinate, propyl acetate), which resulted in mixtures that significantly improved the solubility of tazarotene relative to individual solvent components. As shown in Table 6, tazarotene solubility in compositions comprising 1% to 60% ethanol and 40% to 99% dimethyl succinate is higher than tazarotene solubility in either ethanol or dimethyl succinate alone. As shown in Table 6, tazarotene solubility in compositions comprising 1% to 40% ethanol and 60% to 99% propyl acetate is higher than tazarotene solubility in either ethanol or propyl acetate alone. Thus, the tazarotene in selected ranges of binary mixtures was higher than the solubility of tazarotene in the individual components of the solvent mixture.

TABLE 6
Solubility of Tazarotene in Exemplary Binary
Mixtures of Ethanol and Carboxylate Esters

| | Mixture ratio | Tazarotene |
|---|---|---|
| Solvents | (W/w) | Solubility (mg/g) |
| ethanol and dimethyl succinate | 80:20 | 45.30 |
| ethanol and dimethyl succinate | 60:40 | 67.18 |
| ethanol and dimethyl succinate | 40:60 | 103.65 |
| ethanol and dimethyl succinate | 20:80 | 109.42 |
| ethanol and propyl acetate | 80:20 | 44.12 |
| ethanol and propyl acetate | 60:40 | 80.16 |
| ethanol and propyl acetate | 40:60 | 139.85 |
| ethanol and propyl acetate | 20:80 | 179.99 |

### Example 3A

### Stability of Tazarotene in Exemplary Compositions

Retinoids can undergo chemical degradation in solutions due to oxidation. However, many antioxidants are not effective in sufficiently stabilizing retinoids in a pharmaceutical composition. A study was performed in which samples consisting of 0.05% (w/w) tazarotene dissolved in 79.15% (w/w) ethanol (anhydrous), 0.6% hydroxypropyl cellulose HF (KLUCEL HF), 20% propylene glycol, and 0.20% (w/w) of an antioxidant were stored in a sealed amber glass container for 8 weeks at 40° C. For the tested composition with no antioxidant, the additional 0.20% was made up with ethanol (anhydrous).

The resulting samples were assayed for tazarotene and the results are presented in Table 7. Only one of the antioxidants improved the stability of tazarotene: propyl gallete, reducing the degradation from 5% to 1% over the 8-week period. In contrast, the antioxidant ascorbic acid increased the degradation from 5% to approximately 50% over the 8-week period.

TABLE 7
Solubility of Tazarotene in Compositions
with Selected Antioxidants

| | Reduction in |
|---|---|
| | Tazarotene concentration |
| | after 8 weeks at 40° C. |
| | (% degradation relative |
| Antioxidant (% w/w) | to baseline tazarotene) |
| none | 5% |
| 0.20% propyl gallete | 1% |
| 0.20% tocopherol | 5% |
| 0.20% BHT | 5% |
| 0.20% Dl-Alpha Tocopherol | 5% |
| 0.20% sodium metabisulfite | 8% |
| (SMBS) | |
| 0.20% sodium sulfite | 10% |
| 0.20% sodium nitrate | 10% |
| 0.20% ascorbic acid | ~50% |

Similarly, divalent cations such as magnesium, are also ineffective in stabilizing tazarotene. A mixture comprising ethanol, 0.6% magnesium chloride, and 0.05% tazarotene was stored in a sealed amber glass container for 13 weeks at 40° C. The inclusion of magnesium chloride in this composition showed more degradation than the sample without magnesium chloride. The tazarotene with magnesium chloride degraded by 13% over the 13-week period in comparison to 5% degradation for a comparable mixture without magnesium chloride over the same period.

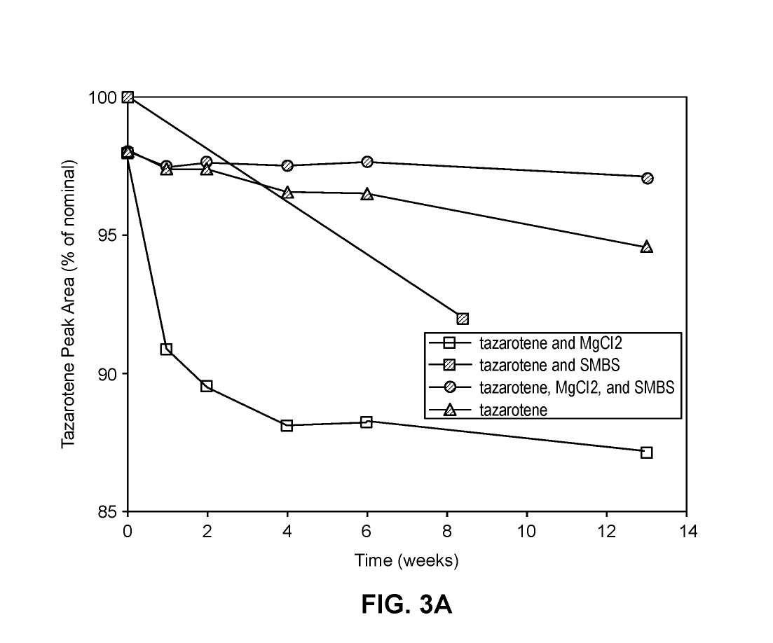

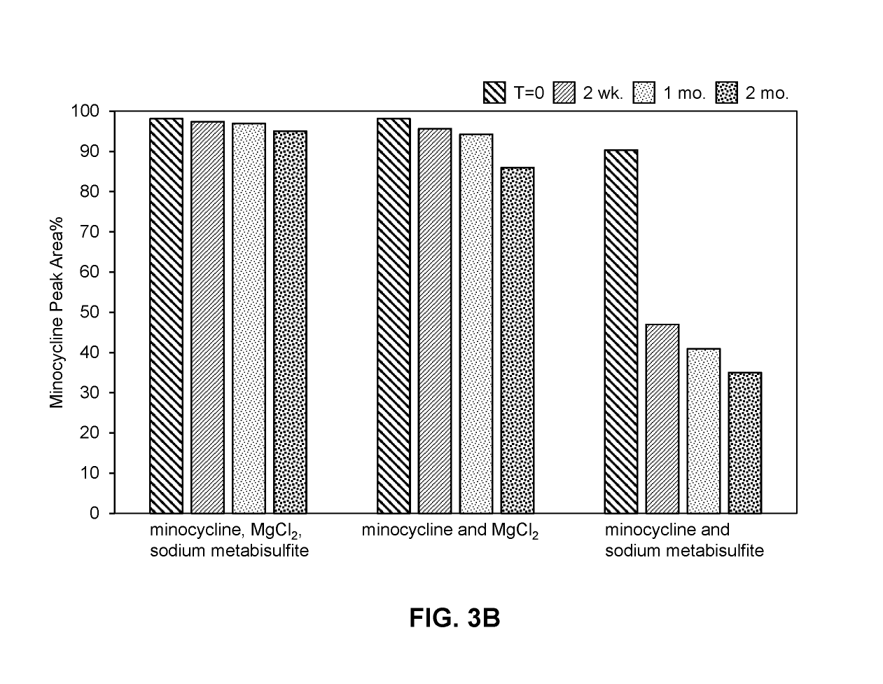

Surprisingly, the combination of a divalent cation (in the form of magnesium chloride) and an antioxidant make tazarotene more stable. A mixture comprising ethanol, 0.6% magnesium chloride, 0.2% sodium metabisulfite, and 0.05% tazarotene was stored in a sealed amber glass container for 13 weeks at 40° C. The tazarotene degraded by 3% over the 13-week period in comparison to 5% degradation for a mixture comprising ethanol and 0.05% tazarotene (and without magnesium chloride or an antioxidant) over the same period. Thus, the combination of magnesium chloride and sodium metabisulfite makes tazarotene in ethanol more stable than tazarotene in ethanol alone even though magnesium chloride or sodium metabisulfite individually in ethanol make tazarotene less stable. These results are shown in FIG. 3A. Measurements for an additional composition comprising ethanol, 0.20% sodium metabisulfite, and 0.05% tazarotene (without the presence of magnesium chloride) were taken at 0, 1, 2, 4, and 8 weeks. Only the data at 0 and 8 weeks are presented in FIG. 3A for this composition. Data for this composition was not captured at 13 weeks.

Tazarotene in a composition comprising ethanol (anhydrous), magnesium chloride, and sodium metabisulfite has been shown to degrade less than 7% when stored for 12 months at room temperature in a sealed amber glass container.

### Example 3B

### Stability of Minocycline in Exemplary Compositions

Tetracycline-class drugs can undergo chemical degradation in solutions due to oxidation and or epimerization. However, many antioxidants are not effective in sufficiently stabilizing tetracycline-class drugs in a pharmaceutical composition. For example, a mixture comprising ethanol, 1.20% minocycline, and 0.20% sodium metabisulfite was stored in a sealed amber glass container for 2-months at 40° C. Measurements were taken at 1-day, 2-week, 1-month, and 2-month time points. The inclusion of the antioxidant sodium metabisulfite in this composition showed significant degradation. The minocycline with the sodium metabisulfite degraded by approximately 55% over the 2-month period.

Similarly, divalent cations such as magnesium are also ineffective in stabilizing minocycline. A mixture comprising ethanol, 1.20% magnesium chloride, and 1.20% minocycline was stored in a sealed amber glass container for 2-months at 40° C. Measurements were taken at 1-day, 2-week, 1-month, and 2-month time points. The inclusion of magnesium chloride in this composition showed degradation. The minocycline with magnesium chloride degraded by approximately 12% over the 2-month period. Surprisingly, the combination of a divalent cation (in the form of magnesium chloride) and an antioxidant make minocycline more stable. A mixture comprising ethanol, 1.20% magnesium chloride, 0.2% sodium metabisulfite, and 1.20% minocycline was stored in a sealed amber glass container for 2-months at 40° C. Measurements were taken at 1-day, 2-week, 1-month, and 2-month time points. The minocycline degraded by approximately 3% over the 2-month period. Thus, the combination of magnesium chloride and sodium metabisulfite makes minocycline in ethanol more stable. These results are shown in FIG. 3B.

### Example 4

### Skin Penetration for Tazarotene

Penetration experiments with ex vivo human skin tissue were conducted to compare the penetration of tazarotene in exemplary compositions to two concentrations of a commercially marketed topical tazarotene composition (TAZORAC cream 0.05% and TAZORAC gel 0.10%). The comparison followed the method described in Example 1. The exemplary compositions that were compared to the TAZORAC compositions are described in Table 8.

TABLE 8
Compositions for Skin Penetration
Comparison to Tazorac Compositions

| COMPOSITION | | |
|---|---|---|
| COMPONENT | TAZ-B | TAZ-A |
| tazarotene | 0.03% | 0.05% |
| hydroxypropyl | 0.6% | 0.6% |
| cellulose HF | | |
| magnesium chloride, | 0.6% | 0.6% |
| anhydrous | | |
| ethanol, anhydrous | 77.57% | 77.55% |
| propylene glycol | 20% | 20% |
| cineole | 1% | 1% |
| sodium metabisulfite | 0.2% | 0.2% |

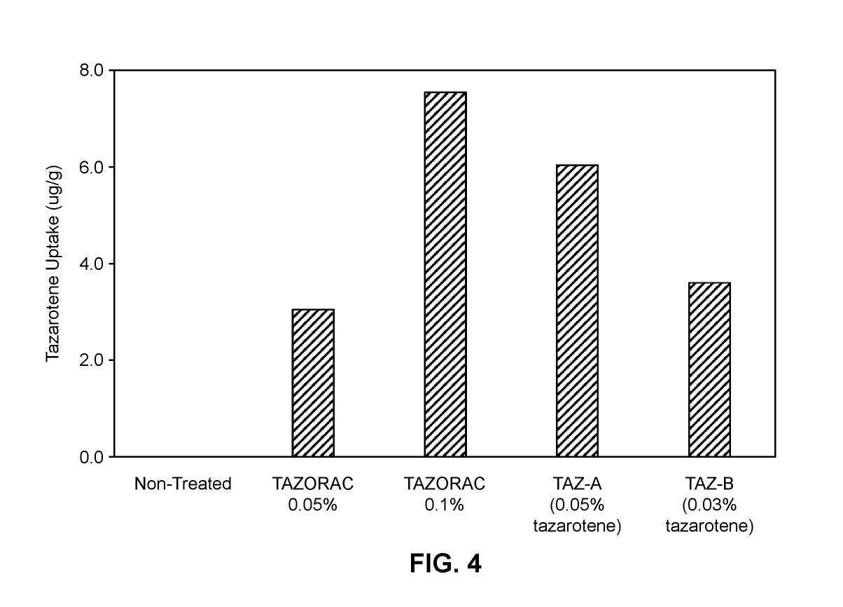

The amount of tazarotene that was measured within the skin samples for each of the tested compositions is presented in FIG. 4. The amount of tazarotene that penetrated into the skin for the 0.05% tazarotene exemplary composition was approximately double the amount of tazarotene that penetrated into the skin for the TAZORAC cream with 0.05% tazarotene and only slightly less than that for the TAZORAC gel with 0.10% tazarotene. Slightly more tazarotene was measured in the 0.03% tazarotene exemplary composition than in the TAZORAC cream with 0.05% tazarotene despite the higher concentration of tazarotene in the TAZORAC cream. Thus, the exemplary compositions more efficiently deliver tazarotene into the skin.

### Example 5

### Stability of a Selective Retinoid and a Tetracycline-Class Antibiotic in Topical Compositions Comprising Both Active Ingredients

The stability of a selective retinoid and a tetracycline-class antibiotic in a single topical composition was evaluated and compared to the corresponding stability for compositions which contained only one of the two active ingredients. The exemplary compositions included tazarotene, minocycline, or a combination thereof. Each composition was stored at room temperature for 12 months in a sealed amber glass container. The exemplary compositions that were tested are described in Table 9.

TABLE 9
Compositions for Skin Penetration Comparison

| COMPOSITION COMPONENT | TAZ-B | TAZ-A | MNC-A | COMBO-B | COMBO-A |
|---|---|---|---|---|---|
| tazarotene | 0.03% | 0.05% | — | 0.03% | 0.05% |
| minocycline | — | — | 0.6% | 0.6% | 0.6% |
| hydroxypropyl cellulose HF | 0.6% | 0.6% | 0.6% | 0.6% | 0.6% |
| magnesium chloride, anhydrous | 0.6% | 0.6% | 0.6% | 0.6% | 0.6% |
| ethanol, anhydrous | 77.57% | 77.55% | 77.00% | 76.97% | 76.95% |
| propylene glycol | 20% | 20% | 20% | 20% | 20% |
| cineole | 1% | 1% | 1% | 1% | 1% |
| sodium metabisulfite | 0.2% | 0.2% | 0.2% | 0.2% | 0.2% |

As shown in FIGS. 5A-5B, both tazarotene and minocycline were stable after 18 months. FIG. 5A shows the stability of tazarotene as produced (designated at T=0), and after storage for 6, 12 and 18 months for each of the tested compositions that comprised tazarotene. FIG. 5B shows the stability of minocycline as produced, and after storage for 6, 12 and 18 months for each of the tested compositions that comprised minocycline. Thus, both tazarotene and minocycline are stable in the exemplary ethanol-based compositions and the two active ingredients are chemical compatible.

Surprisingly, the results presented in FIGS. 5A-5B show that both tazarotene and minocycline were more stable after a period of 18 months when added in combination than when each drug was added alone. Thus, the presence of tazarotene improved the stability of minocycline and the presence of minocycline improved the stability of tazarotene as shown by comparison of the compositions described in Table 9.

### Example 6

### Topical Uptake of Selective Retinoid and a Tetracycline-Class Antibiotic in Topical Compositions Comprising Both Active Ingredients

Penetration experiments with ex vivo human skin tissue were conducted to compare the penetration of tazarotene and minocycline in exemplary compositions where tazarotene and minocycline were combined within a single formulation to penetration of formulations that used only one of these drugs. Each of the exemplary compositions included tazarotene, minocycline, or a combination thereof. The comparison followed the method described in Example 1. The exemplary compositions that were tested are described in Table 9.

The amount of tazarotene and minocycline that was measured within the skin samples for each of the tested compositions is presented in FIGS. 6A-6B. As shown in FIG. 6A, addition of minocycline to the tazarotene composition does not inhibit the tazarotene uptake. As shown in FIG. 6B, the addition of tazarotene to minocycline composition does not inhibit the minocycline uptake. Thus, this experiment showed that combining these two drugs in the same composition does not inhibit the topical penetration of either in human skin.

### Example 7

### Minocycline Penetration into Ex Vivo Human Skin from Minocycline Compositions

Penetration experiments with ex vivo human skin tissue were conducted to determine whether minocycline penetrates into the skin in sufficient concentrations to achieve a desired therapeutic effect when comprised within compositions that are applied to the skin surface and the composition comprises a monohydric aliphatic alcohol, a polyol, a carboxylate ester, a magnesium salt, and a sulfite. The penetration into facial skin was assessed for three different human donors with two samples from each donor for each data point.

Solvent mixtures were prepared in the proportions described in Table 10, with each solvent mixture comprising anhydrous ethanol (Spectrum Chemicals, Gardena, Calif.), propylene glycol (Spectrum Chemicals, Gardena, Calif.) or glycerol (Spectrum Chemicals, Gardena, Calif.), and a selected carboxylate ester. To each solvent mixture was added 1.2% (w/w) minocycline hydrochloride (Euticals S.P.A, Origgio, Italy) (1.0% base equivalent), 1.2% (w/w) magnesium chloride (anhydrous) (Sigma-Aldrich Corp., St. Louis, Mo.), 0.20% sodium metabisulfite (Spectrum Chemicals, Gardena, Calif.), and 0.60% (w/w) hydroxypropyl cellulose HF (KLUCEL HF, Ashland, Inc., Covington, Ky.).

TABLE 10
Compositions for Skin Penetration Study with Varied Solvent Systems

| COMPOSITION | | | | | |
|---|---|---|---|---|---|
| COMPONENT | MNC-C | MNC-C.1 | MNC-C.2 | MNC-C.3 | |
| minocycline | 1.20% | 1.20% | 1.20% | 1.20% | |
| hydrochloride | | | | | |
| hydroxypropyl | 0.60% | 0.60% | 0.60% | 0.60% | |
| cellulose HF | | | | | |
| magnesium chloride, | 1.20% | 1.20% | 1.20% | 1.20% | |
| anhydrous | | | | | |
| ethanol, anhydrous | 75.80% | 65.80% | 65.80% | 65.80% | |
| propylene glycol | 20.00% | 20.00% | 20.00% | 20.00% | |
| cineole | 1.00% | 1.00% | 1.00% | 1.00% | |
| Kollisolv MCT 70 | — | 10.00% | — | — | |
| isopropyl myristate | — | — | 10.00% | — | |
| diisopropyl adipate | — | — | — | 10.00% | |
| sodium metabisulfite | 0.20% | 0.20% | 0.20% | 0.20% | |
| COMPOSITION | MNC- | MNC- | MNC- | MNC- | MNC- |
| COMPONENT | C.1.1 | C.3.1 | C.2.3 | C.6.1 | C.6.2 |
| minocycline | 1.20% | 1.20% | 1.20% | 1.20% | 1.20% |
| hydrochloride | | | | | |
| hydroxypropyl | 0.60% | 0.60% | 0.60% | 0.60% | 0.60% |
| cellulose HF | | | | | |
| magnesium chloride, | 1.20% | 1.20% | 1.20% | 1.20% | 1.20% |
| anhydrous | | | | | |
| ethanol, anhydrous | 75.80% | 75.80% | 60.05% | 75.80% | 60.05% |
| propylene glycol | 10.00% | 10.00% | 15.75% | | |
| glycerol | — | — | — | 10.00% | 15.75% |
| cineole | 1.00% | 1.00% | 1.00% | 1.00% | 1.00% |
| Kollisolv MCT 70 | 10.00% | — | — | — | — |
| isopropyl myristate | — | — | 20.00% | 10.00% | 20.00% |
| diisopropyl adipate | — | 10.00% | — | — | — |
| sodium metabisulfite | 0.20% | 0.20% | 0.20% | 0.20% | 0.20% |

| COMPOSITION | MNC- | MNC- | MNC- | MNC- |
|---|---|---|---|---|
| COMPONENT | C.7.2 | C.9.1 | C.11.1 | C.12.1 |
| minocycline | 1.20% | 1.20% | 1.20% | 1.20% |
| hydrochloride | | | | |
| hydroxypropyl | 0.60% | 0.60% | 0.60% | 0.60% |
| cellulose HF | | | | |
| magnesium chloride, | 1.20% | 1.20% | 1.20% | 1.20% |
| anhydrous | | | | |
| ethanol, anhydrous | 60.05% | 74.23% | 67.90% | 60.05% |
| propylene glycol | 15.75% | 19.57% | 17.90% | — |
| glycerol | — | — | — | 15.75% |
| cineole | 1.00% | 1.00% | 1.00% | 1.00% |
| Ethyl Acetate | 20.00% | — | — | 20.00% |
| AZONE ® | — | 2.00% | — | — |
| Lauryl alcohol | — | — | 10.00% | — |
| (LIPOCOL L) | | | | |
| sodium metabisulfite | 0.20% | 0.20% | 0.20% | 0.20% |

The compositions were applied to skin samples from three human donors at a gel volume of 2.5 mg/cm². Tissue was maintained in a damp environment to limit drying of the tissue and incubated at 32° C. for 4 hours. At the end of the incubation period, excess composition was wiped from the surface using first a dry gauze pad, second a gauze pad soaked with 70% isopropyl alcohol, and finally with a dry gauze pad. Two six (6) millimeter punch biopsies were taken from within each test area. From each of the biopsies, minocycline was extracted from the biopsy using acidified methanol. The supernatants were analyzed by high performance liquid chromatography.

The ratio of the number of ester groups to the number of carbon atoms in the carboxylate ester is presented in Table 3 above (Example 1) for each tested composition and the minocycline uptake is also in Table 3 and in FIG. 1. The results demonstrate that the efficiency of penetration of minocycline increases as the ratio of ester groups to carbon atoms in the carboxylate ester increases.

### Example 8

### Exemplary Embodiments

Table 11 provides illustrative topical compositions. The compositions were prepared by first mixing the solvent components together (e.g., ethanol, propylene glycol, cineole, glycerin, ethyl acetate, and triacetin), then the magnesium compound was separately mixed with the tetracycline-class drug and added to the solvent system with the antioxidant (e.g., sodium metabisulfite) and thickener (e.g. hydroxypropyl cellulose HF), if applicable. As mentioned above, these exemplary compositions are in no way intended to limit the scope of what the inventors regard as their invention.

TABLE 11
Exemplary Compositions

| COMPOSITION | | COMBO- | COMBO- | COMBO- | COMBO- | COMBO- |
|---|---|---|---|---|---|---|
| COMPONENT | COMBO-C | C.12.3 | C.18.2 | C.19.1 | C.20.1 | C.21.1 |
| minocycline | 1.20% | 1.20% | 1.20% | 1.20% | 1.20% | 1.20% |
| hydrochloride | | | | | | |
| tazarotene | 0.05% | 0.05% | 0.05% | 0.05% | 0.05% | 0.05% |
| hydroxypropyl | 0.60% | 0.60% | 0.60% | 0.60% | 0.60% | 0.60% |
| cellulose HF | | | | | | |
| magnesium chloride, | 1.20% | 1.20% | 1.20% | 1.20% | 1.20% | 1.20% |
| anhydrous | | | | | | |
| ethanol, anhydrous | 75.75% | 56.05% | 56.05% | 60.00% | 60.00% | 60.00% |
| propylene glycol | 20.00% | — | 7.35% | 15.75% | 7.88% | — |
| cineole | 1.00% | 1.00% | 1.00% | 1.00% | 1.00% | 1.00% |
| glycerin | — | 14.70% | 7.35% | — | 7.88% | 15.75% |
| ethyl acetate | — | 25.00% | 25.00% | — | — | — |
| triacetin | — | — | — | 20.00% | 20.00% | 20.00% |
| sodium metabisulfite | 0.20% | 0.20% | 0.20% | 0.20% | 0.20% | 0.20% |

Table 12 provides further illustrative topical compositions. Solvent mixtures are prepared in the proportions described in Table 12, with each solvent mixture comprising anhydrous ethanol (Spectrum Chemicals, Gardena, Calif.), propylene glycol (Spectrum Chemicals, Gardena, Calif.), cineole (Penta International, Livingston, N.J.), and a selected carboxylate ester or a combination of selected carboxylate esters. To each solvent mixture is added 1.2% (w/w) minocycline hydrochloride (Euticals S.P.A, Origgio, Italy), 0.05% (w/w) tazarotene (AvaChem Scientific, San Antonio, Tex.), 1.2% (w/w) magnesium chloride (anhydrous) (Magnesium Products, Tulsa, Okla.), 0.20% sodium metabisulfite (Spectrum Chemicals, Gardena, Calif.), and 0.60% (w/w) hydroxypropyl cellulose HF (KLUCEL HF, Ashland, Inc., Covington, Ky.). As mentioned above, these exemplary compositions are in no way intended to limit the scope of what the inventors regard as their invention.

TABLE 12
Additional Exemplary Compositions

| Formulation | Combo- | Combo- | Combo- | Combo- | Combo- |
|---|---|---|---|---|---|
| Number | C.28.5 | C.30.6 | C.30.7 | C.30.8 | C.30.9 |
| Dose, Minocycline | 1.00% | 1.00% | 1.00% | 1.00% | 1.00% |
| Dose, Tazarotene | 0.05% | 0.05% | 0.05% | 0.05% | 0.05% |
| Minocycline HCl | 1.20% | 1.20% | 1.20% | 1.20% | 1.20% |
| Tazarotene | 0.05% | 0.05% | 0.05% | 0.05% | 0.05% |
| Klucel HF | 0.60% | 0.60% | 0.60% | 0.60% | 0.60% |
| Magnesium | 1.20% | 1.20% | 1.20% | 1.20% | 1.20% |
| Chloride Anh. | | | | | |
| Ethanol, anhydrous | 60.00% | 60.00% | 60.00% | 67.85% | 60.00% |
| Propylene Glycol | 15.75% | 15.75% | 15.75% | 17.90% | 15.75% |
| Eucalyptol/Cineole | 1.00% | 1.00% | 1.00% | 1.00% | 1.00% |
| Dimethyl succinate | 20.00% | — | — | — | — |
| Propyl acetate | — | — | — | — | — |
| Amyl Acetate | — | 10.00% | — | — | — |
| Prenyl Acetate | — | — | 10.00% | — | 20.00% |
| Benzyl Acetate | — | 10.00% | 10.00% | — | — |
| Benzyl Propionate | — | — | — | 10.00% | — |
| Sodium | 0.20% | 0.20% | 0.20% | 0.20% | 0.20% |
| Metabisulfite | | | | | |

### Example 9

### Penetration Comparison of Exemplary Embodiments

Penetration experiments with ex vivo human skin tissue were conducted to determine whether minocycline or tazarotene penetrated into the skin in sufficient concentrations to achieve a desired therapeutic effect when comprised within the exemplary compositions described in Table 13-1 below. The comparison follows the method described in Example 1.

TABLE 13-1
Compositions for Penetration and Stability Comparison of Single and Dual Active
Pharmaceutical Ingredient (API) Compositions

| | Combo | Combo | Combo- | | MNC- | MNC- | MNC- | | TAZ- | TAZ- |
|---|---|---|---|---|---|---|---|---|---|---|
| Formulation | C | C.28.1 | C.30.3 | MNC-C | C7.2 | C.28.1 | C.30.3 | TAZ-C | C.28.1 | C.30.3 |
| Minocycline | 1.00% | 1.00% | 1.00% | 1.00% | 1.00% | 1.00% | 1.00% | — | — | — |
| Tazarotene | 0.05% | 0.05% | 0.05% | — | — | — | — | 0.05% | 0.05% | 0.05% |
| | Percent in Formulation | | | | | | | | | |
| Minocycline | 1.20 | 1.20% | 1.20% | 1.20% | 1.20% | 1.20% | 1.20% | — | — | — |
| HCl | | | | | | | | | | |
| Tazarotene | 0.05 | 0.05% | 0.05% | — | — | — | — | 0.05 | 0.05 | 0.05 |
| KLUCEL¹ HF | 0.6 | 0.6 | 0.6 | 0.6 | 0.6 | 0.6 | 0.60 | 0.6 | 0.60 | 0.6 |
| Magnesium | | | | | | | | | | |
| Chloride Anh. | 1.2 | 1.2 | 1.2 | 1.2 | 1.2 | 1.2 | 1.2 | 1.2 | 1.2 | 1.2 |
| Ethanol, | 75.7 | 67.8 | 63.9 | 75.8 | 60 | 67.9 | 64 | 76.9 | 69 | 65 |
| anhydrous | | | | | | | | | | |
| Propylene | 20 | 17.9 | 16.8 | 20 | 15.8 | 17.9 | 16.8 | 20 | 17.9 | 16 |
| Glycol | | | | | | | | | | |
| Eucalyptol | 1 | 1 | 1 | 1 | 1 | 1 | 1 | 1 | 1 | 1 |
| (Cineole) | | | | | | | | | | |
| Ethyl acetate | | | | | 20 | | | | | |
| Dimethyl | — | 10 | 10 | — | | 10 | 10 | — | 10 | 10 |
| succinate | | | | | | | | | | |
| Propyl acetate | — | — | 5 | — | | — | 5 | — | — | 5 |
| Sodium | | | | | | | | | | |
| Metabisulfite | 0.2 | 0.2 | 0.2 | 0.2 | 0.2 | 0.2 | 0.2 | 0.2 | 0.2 | 0.2 |

¹KLUCEL = hydroxypropyl cellulose

Other exemplary compositions are set forth in Table 13-2. Penetration experiments with ex vivo human skin tissue were conducted to determine whether minocycline or tazarotene penetrated into the skin in sufficient concentrations to achieve a desired therapeutic effect when comprised within the exemplary compositions described in Table 13-2 below. The comparison follows the method described in Example 1

TABLE 13-2
Compositions for Penetration and Stability Comparison of Single
and Dual Active Pharmaceutical Ingredient (API) Compositions

| | Combo- | MNC- | TAZ- |
|---|---|---|---|
| | C.28.2 | C.28.2 | C.28.2 |
| Dose, Minocycline | 1.00% | 1.00% | — |
| Dose, Tazarotene | 0.05% | — | 0.05% |
| | Percent in Formulation | | |
| Minocycline HCl | 1.19% | 1.19% | — |
| Tazarotene | 0.05% | — | 0.05% |
| Klucel HF | 0.60% | 0.60% | 0.60% |
| Magnesium Chloride Anh. | 1.20% | 1.20% | 1.20% |
| Ethanol, anhydrous | 68.11% | 68.13% | 69.03% |
| Propylene Glycol | 17.90% | 17.93% | 18.17% |
| Eucalyptol/Cineole | 0.75% | 0.75% | 0.75% |
| Dimethyl succinate | 10.00% | 10.00% | 10.00% |
| Propyl acetate | — | — | — |
| Sodium Metabisulfite | 0.20% | 0.20% | 0.20% |

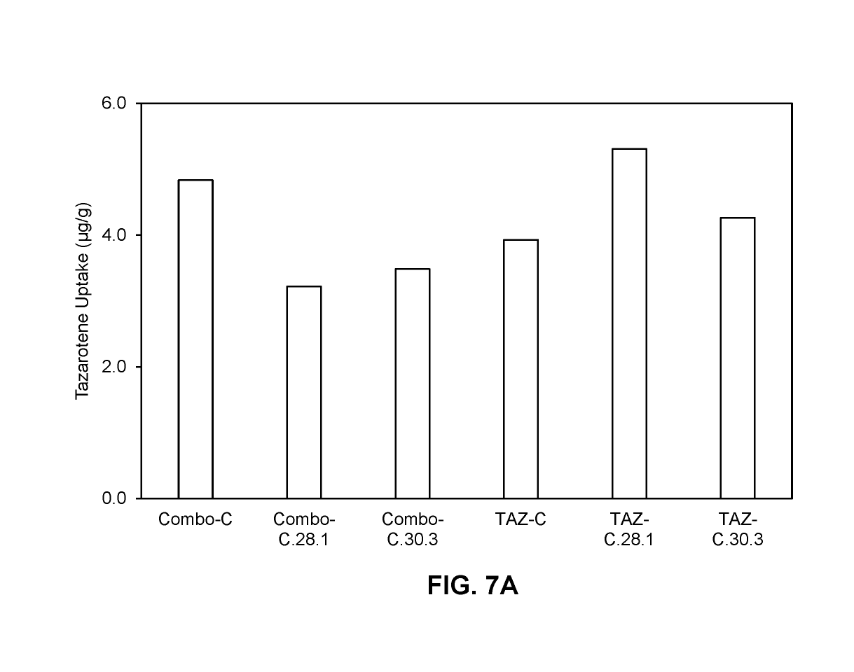

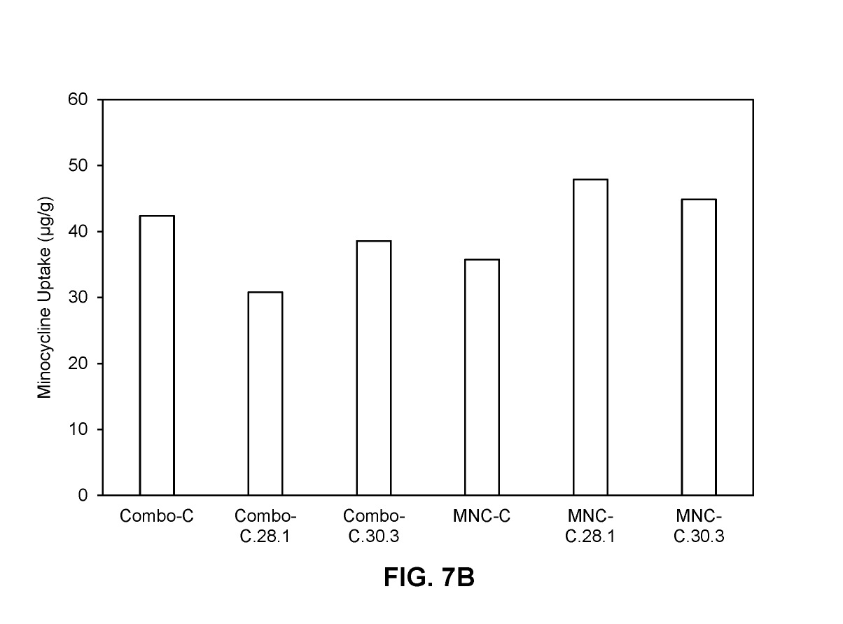

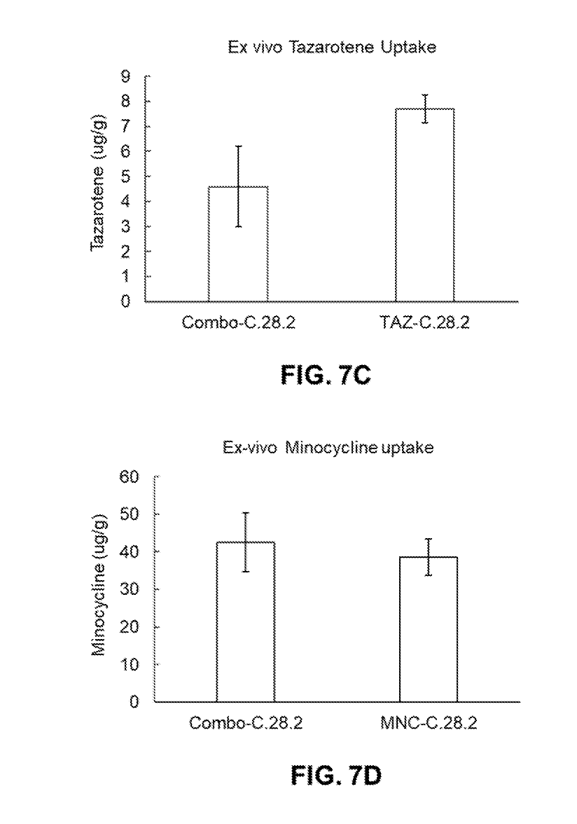

The average values of tazarotene and minocycline from the six donor skin samples of each of the tested compositions are presented in FIGS. 7A-7D. The results demonstrated that the combination of both minocycline and tazarotene does not inhibit the efficiency of topical penetration of either drug (active pharmaceutical ingredient or API) in human skin. The results also demonstrated the efficiency of minocycline topical penetration into human skin and the efficiency of tazaratene topical penetration into human skin from the compositions comprising these drugs alone.

### Example 10

### Stability Comparison of Exemplary Embodiments

The degradation and stability of a selective retinoid and a tetracycline-class antibiotic in a single topical composition was evaluated and compared to the corresponding stability for compositions which contained only one of the two active ingredients. The exemplary compositions comprised tazarotene, minocycline, or a combination thereof. Each composition was stored at room temperature for 2 and 5 weeks in a sealed amber glass container at 40° C. The exemplary compositions that were tested are described in Table 13-1 above.

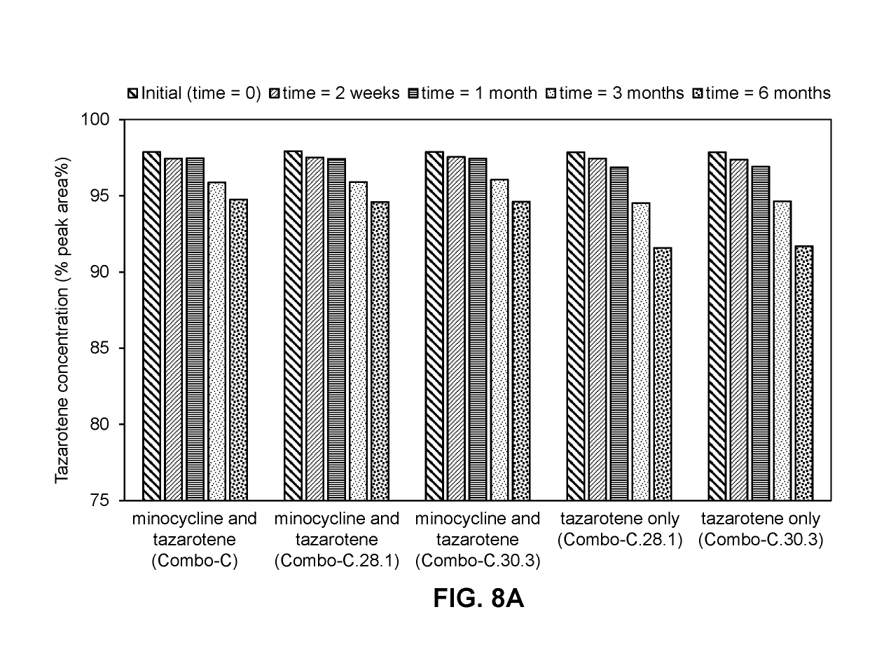

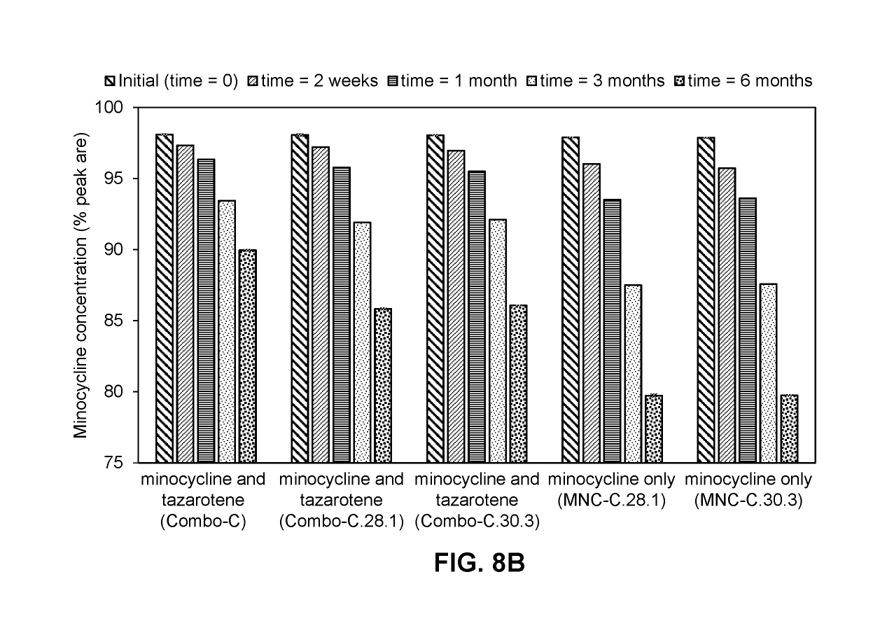

As shown in FIGS. 8A-8B, both tazarotene and minocycline were stable in both single API compositions and dual API compositions after a period of 6 months. FIG. 8A shows the stability of tazarotene as produced (designated as T=0), and after storage for up to 6 months for each of the tested compositions that comprised tazarotene. FIG. 8B shows the stability of minocycline as produced and after storage for up to 6 months for each of the tested compositions that comprised minocycline.

Surprisingly, the results presented in FIGS. 8A-8B showed that both tazarotene and minocycline were more stable after a period of 6 months when added in combination than when each was added alone. Thus, the presence of tazarotene improved the stability of minocycline and the presence of minocycline improved the stability of tazarotene in the compositions described in Table 13-1.

### Example 11

### In Vivo P. acnes-Bacteria-Triggered Inflammatory Model

Two of the four key factors in the pathogenesis of acne in humans are the proliferation of P. acnes in the sebaceous gland and increased inflammation. The clinical effect of the inventive composition was simulated in an animal study in which P. acnes bacteria were injected into the skin of mice to create acne-like lesions. Following these injections, compositions as described in Table 14 were topically applied as well as control compositions. Resulting measurements were compared to identify the relative efficacy in lesion reduction.

Live P. acnes (1×10⁸ CFU/μL) bacteria in phosphate-buffered saline (PBS) were injected intradermally into the dorsal area in the region of the shoulders and upper back of 7-week-old, male SKH-1 hairless mice. On Day −2, four single injections were performed in each mouse to form four separate lesions on the back of each mouse. Two days after the P. acnes injection, i.e., on Day 0, the width of each lesion was measured with a caliper and the height was measured with a ruler. Lesion volumes were calculated assuming cylindrically symmetrical lesions using an oblate semi-ellipsoid formula. The mice were then randomly divided into treatment groups and a non-treatment group, with eight mice per group.

Starting on Day 0 after the lesion measurement, each treatment group received a daily topical application of a selected test composition as described in Table 14. For each mouse, a test composition application amount of 2.5 mg/cm² skin was evenly applied daily over an area of approximately 8 cm².

TABLE 14
Test Composition Formulations

| COMPOSITION | | | |
|---|---|---|---|
| COMPONENT | VEHICLE | COMBO-C | COMBO-D |
| minocycline hydrochloride | — | 1.20% | 1.20% |
| tazarotene | — | 0.05% | 0.03% |
| hydroxypropyl cellulose HF | 0.60% | 0.60% | 0.60% |
| magnesium chloride, | 0.6% | 1.20% | 1.20% |
| anhydrous | | | |
| ethanol, anhydrous | 77.59% | 75.75% | 75.77% |
| propylene glycol | 20% | 20% | 20% |
| glycerol | — | — | — |
| cineole | 1% | 1% | 1% |
| sodium metabisulfite | 0.20% | 0.20% | 0.20% |
| color additive | 0.01% | | |

On Day 14, individual lesions were remeasured as described above and lesion volumes were normalized to the corresponding lesion volume on Day 0. Results from the normalized lesion volumes at Day 14 are shown in Table 15. As can be seen by the results, the combination treatment did reduce the lesion volume more than did either the vehicle or no treatment groups.

TABLE 15
Percent change in Lesion Volume after 14 days of treatment
relative to lesion volume prior to treatment

| | CHANGE IN LESION VOLUME |
|---|---|
| COMPOSITION | (DAY 14 NORMALIZED TO DAY 1 |
| APPLIED | PRIOR TO FIRST TREATMENT) |
| No treatment | −1% |
| Vehicle | −26% |
| Combo C | −75% |
| Combo D | −61% |

### Example 12

### Stability of Adapalene and Minocycline in Exemplary Compositions

A study was performed in which samples comprising adapalene and/or minocycline were dissolved in the compositions as described in Table 16. These samples were stored in sealed amber glass containers for 8 weeks at 40° C. The resulting samples were assayed for adapalene and/or minocycline. Both combinations tested showed good retention of the active ingredients after this 8-week accelerated aging test.

TABLE 16
Exemplary Compositions for Evaluating Stability of Adapalene
and Minocycline (w/w)

| COM- | | | | | | |
|---|---|---|---|---|---|---|
| POSITION | | | | | | |
| COM- | MNC- | MNC- | ADA- | ADA- | COMBO- | COMBO- |
| PONENT | F | G | F | G | F | G |
| minocycline | 1.00% | 0.60% | — | — | 1.00% | 0.60% |
| hydrochloride | | | | | | |
| adapalene | — | — | 0.10% | 0.20% | 0.10% | 0.20% |
| hydroxy- | 0.60% | 0.60% | 0.60% | 0.60% | 0.60% | 0.60% |
| propyl | | | | | | |
| cellulose HF | | | | | | |
| magnesium | 1.20% | 0.60% | 1.20% | 0.60% | 1.20% | 0.60% |
| chloride, | | | | | | |
| anhydrous | | | | | | |
| ethanol, | 47.0% | 28.0% | 47.9% | 28.4% | 46.9% | 27.8% |
| anhydrous | | | | | | |
| propylene | 20.0% | 20.0% | 20.0% | 20.0% | 20.0% | 20.0% |
| glycol | | | | | | |
| cineole | 30.0% | 50.0% | 30.0% | 50.0% | 30.0% | 50.0% |
| sodium | 0.20% | 0.20% | 0.20% | 0.20% | 0.20% | 0.20% |
| metabisulfite | | | | | | |

TABLE 17
Stability of Adapalene and Minocycline
in Exemplary Compositions

| | ADAPALENE | MINOCYCLINE |
|---|---|---|
| | CONCENTRATION | CONCENTRATION |
| | (% PEAK AREA) | (% PEAK AREA) |
| | AFTER 8 | AFTER 8 |
| | WEEKS AT | WEEKS AT |
| COMPOSITION | 40° C. | 40° C. |
| MNC-F | — | 93.8 |
| MNC-G | — | 92.3 |
| ADA-F | 95.5 | — |
| ADA-G | 96.9 | — |
| COMBO-F | 90.0 | 93.0 |
| COMBO-G | 95.3 | 88.8 |

### Example 13

### Solubility of Antioxidant in the Presence of Minocycline

The solubility of antioxidant in the presence of minocycline was evaluated. Sodium metabisulfite and sodium thiosulfate are exemplary effective antioxidants for minocycline. Sodium sulfate is an exemplary non-antioxidant for minocycline. Each set of solubility tests was done in all four compositions with the same vehicle containing anhydrous magnesium chloride, anhydrous ethanol, propylene glycol and cineole at the same weight ratio of 1.2 to 75.8 to 20 to 1, and with only difference in minocycline content. Four test compositions were evaluated: a control without minocycline HCl, 1.20%, 2.36% and 3.54% minocycline hydrochloride, respectively. The solubility of the antioxidants sodium metabisulfite and sodium thiosulfate and of the non-antioxidant sodium sulfate in the compositions was determine. Results are in Table 18.

TABLE 18
Solubility of Antioxidant in the Presence of Minocycline

| | Solubility (mg/g) | | | |
|---|---|---|---|---|
| | no | 1% | 2% | 3% |
| | mino- | mino- | mino- | mino- |
| | cycline | cycline | cycline | cycline |
| sodium metabisulfite | 0.32 | 3.12 | 3.21 | 3.32 |
| sodium thiosufate | 0.29 | 2.53 | 2.71 | 2.85 |
| sodium sulfate (control, | 0.23 | 0.80 | 0.89 | 1.07 |
| non-antioxidant) | | | | |

As seen in Table 18, the solubility of sodium metabisulfite was greatly improved—from 9 to 10-fold—by the presence of between about 1-3 wt % minocycline. The solubility of sodium thiosulfate was improved 8 to 10-fold by the presence of between about 1-3% minocycline. The solubility of non-antioxidant control sodium sulfate was improved by 3 to 4-fold by the presence of between about 1-3% minocycline. Minocycline was stabilized in the exemplary compositions containing sodium metabisulfite or sodium thiosulfate. The positive correlation of sodium metabisulfite and sodium thiosulfate solubility with minocycline concentration indicates possible interaction of sodium metabisulfite and sodium thiosulfate with minocycline in the exemplary compositions.

### Example 14

### Solubility of Minocycline in the Presence of Magnesium Chloride

The solubility of minocycline in the presence of magnesium chloride, in ethanol, is relatively good at about 130 mg/g. However, compositions with ethanol as the only solvent are typically irritating to the skin, have a pungent smell, and leave the skin feeling dry. Aspects of the solvent system can be desirably improved by adding other solvents while maintaining sufficient solubility of minocycline. Mixtures of ethanol and carboxylate esters have higher solubility of minocycline than would be expected from mixtures of the carboxylate esters individually, as this study demonstrates.

As shown in Table 19, carboxylate esters on their own do not demonstrate good solubility for minocycline in the presence of magnesium chloride. Minocycline is desirably used in compositions with concentrations of 0.1% to 5.0% (w/w). So, solubilities of at least about 1.0 to 50.0 mg/g are required to solubilize minocycline. However, significantly higher solubilities are generally desirable so that the minocycline does not precipitate out of solution as temperature is varied and so that minocycline will remain in solution for a longer period after the composition is applied to the skin and the solvent evaporates or penetrates into the skin. For these reasons, minocycline solubility of at least 10 mg/g is desired, preferably at least 20 mg/g, at least 30 mg/g, at least 40 mg/g, or at least 50 mg/g.

TABLE 19
Solubility of Minocycline in Individual Solvents

| | Minocycline Solubility (mg/g) |
|---|---|
| Solvent | in the presence of MgCl₂ |
| ethanol | 129.3 |
| propylene glycol | 126.0 |
| isopropyl myristate | <0.1 |
| diisopropyl adipate | <0.1 |
| medium chain triglycerides | <0.1 |

The solubility of minocycline was significantly improved by selected binary and tertiary mixtures of ethanol and a carboxylate ester as shown in Table 20. The minocycline solubility in selected binary and tertiary mixtures was even higher than the solubility of minocycline in the ethanol or carboxylate ester alone. Even small amounts of carboxylate esters can have unexpectedly large effects. For example, the solubility of minocycline in the mixture ratio (w/w) 7.5:1 of ethanol and isopropyl myristate is 35.1 mg/g compared to only <0.1 mg/g in isopropyl myristate alone.

TABLE 20
Solubility of Minocycline in Binary
and Tertiary Solvent Mixtures

| | | Minocycline |
|---|---|---|
| | | Solubility |
| | | (mg/g) |
| | Mixture | in the |
| | ratio | presence |
| Solvents | (w/w) | of MgCl₂ |
| ethanol and isopropyl myristate | 7.5:1 | 35.1 |
| propylene glycol and isopropyl myristate | 2:1 | 2.8 |
| propylene glycol and diisopropyl adipate | 2:10 | 2.3 |
| ethanol, propylene glycol, and isopropyl | 7.5:2:1 | 16.2 |
| myristate | | |
| ethanol, propylene glycol, and | 7.5:2:1 | 18.2 |
| diisopropyl adipate | | |
| ethanol, propylene glycol, and medium | 7.5:2:1 | 15.8 |
| chain triglycerides | | |
| ethanol, propylene glycol, and medium | 3.3:2:1 | 9.3 |
| chain triglycerides | | |
| ethanol, glycerol, and isopropyl myristate | 7.5:2:1 | 32.6 |

The combination of ethanol with carboxylate esters (e.g. isopropyl myristate, diisopropyl adipate, and medium chain triglycerides) resulted in unique mixtures that significantly improved the solubility of minocycline. Thus, the mixtures comprising ethanol and a carboxylate ester were shown to form a solvent with beneficial and unexpected characteristics relative to the individual components. This increased solubility was not seen in mixtures of ethanol and propylene glycol or in mixtures of propylene glycol and a carboxylate ester (e.g. isopropyl myristate and diisopropyl adipate). Instead, these mixtures followed expected behavior for such binary mixtures of solvent components.

### Example 15

### Stability of Minocycline in Exemplary Compositions

A study was performed in which samples comprising minocycline were dissolved in the compositions MNC-C, MNC-C7.2, MNC-C.28.1 and MNC-C.30.3 of Table 21. The samples were stored in sealed amber glass containers for 3 months and 6 months at 40° C.

TABLE 21
Stability of Minocycline

| | Minocycline | Minocycline |
|---|---|---|
| | concentration (% | concentration (% |
| | Peak Area) | Peak Area) |
| | after 3 Months at | after 6 Months at |
| Composition | 40° C. | 40° C. |
| MNC-C | 94.25% | 90.21% |
| MNC-C.7.2 | 93.12% | 88.15% |
| MNC-C.28.1 | 87.50% | — |
| MNC-C.30.3 | 87.58% | — |

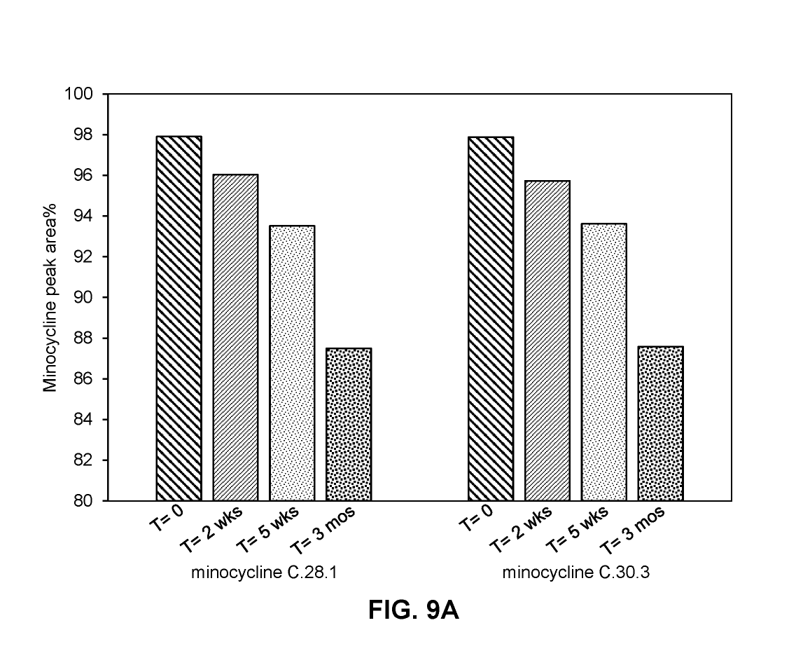

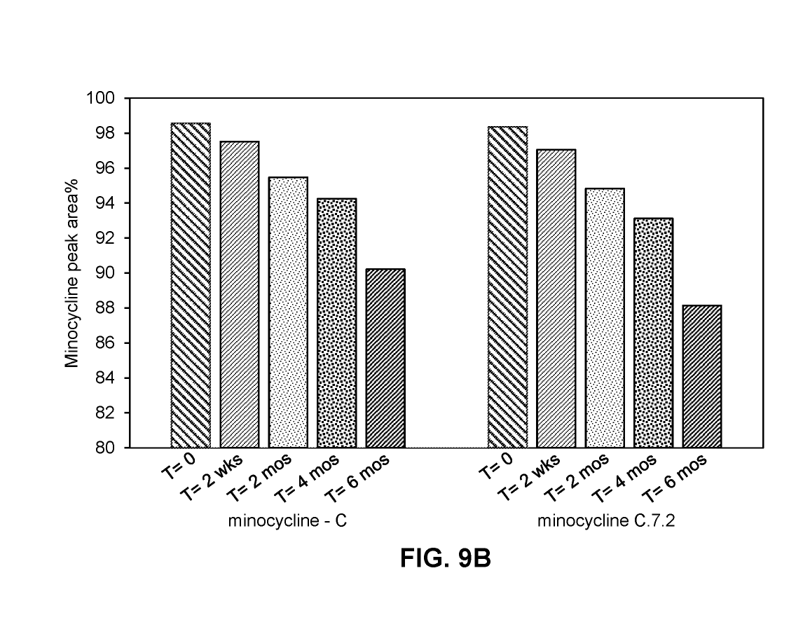

The resulting samples were assayed for minocycline. Table 21 above shows the stability of minocycline after storage for 3 months and 6 months for each of the tested compositions that comprised minocycline. FIG. 9A shows the stability of minocycline as produced (designated at T=0), and after storage for 3 months for the tested compositions MNC-C.28.1 and MNC-C.30.3, that both comprise minocycline. FIG. 9B shows the stability of minocycline as produced (designated at T=0), and after storage for 6 months for the tested compositions MNC-C and MNC-C.7.2, that both comprise minocycline. As shown in Table 21 above, and in FIGS. 9A-9B, the combinations tested showed good retention of the minocycline after a period of 3 months and 6 months at 40° C.

### Example 16

### Improved Stability of Minocycline and Magnesium-Stabilized Minocycline by Antioxidants

The effect of the selection of antioxidant on drug potency, stability and epimer formation was evaluated for illustrative compositions. The degradation and stability of minocycline in compositions were evaluated at baseline and after storage for 1, 2, and 4 weeks in the dark at 40° C. within sealed glass vials. Efforts were taken to minimize (to the extent practical in a typical lab environment) the amount of empty space in the vial above each composition to reduce the interaction between the composition and any water vapor in the air.

Degradation of minocycline to its epimer was quantified by evaluating the change in the relative concentration of 4-epi-minocycline, which was calculated as the 4-epi-minocycline peak area divided by the active minocycline peak area. As a separate quantification, stability was quantified by evaluating the change in the relative concentration of active minocycline, which was calculated as the active minocycline peak divided by the sum of the peak areas for all peaks observed in the HPLC chromatograph. For these measurements, a runtime of 20 minutes was used for the HPLC.

Each of the compositions evaluated in the study described in this example includes the materials described in Table 22. The first six materials, i.e., minocycline hydrochloride, hydroxypropyl cellulose, magnesium chloride (anhydrous), ethanol (anhydrous), propylene glycol, and 1,8-cineole make up 99.8-100% of each of the evaluated compositions. The remaining 0.2% consisted of one of the antioxidants listed in Table 10 or consisted of ethanol (anhydrous) as a control (19-Ctrl) or non-antioxidant control (51-ss).

TABLE 22
Exemplary Compositions for Evaluating Stability of Minocycline (w/w)

| Composition | | | | | | |
|---|---|---|---|---|---|---|
| Component | 1-sbs | 27-st.2 | 52-pt | 53-at | 51-ss | 19-Ctrl |
| minocycline | 1.20% | 1.20% | 1.20% | 1.20% | 1.20% | 1.20% |
| hydrochloride | | | | | | |
| hydroxypropyl | 0.60% | 0.60% | 0.60% | 0.60% | 0.60% | 0.60% |
| cellulose HF | | | | | | |
| magnesium | 1.20% | 1.20% | 1.20% | 1.20% | 1.20% | 1.20% |
| chloride, | | | | | | |
| anhydrous | | | | | | |
| ethanol, | 75.80% | 75.80% | 75.95% | 75.80% | 75.80% | 76.00% |
| anhydrous | | | | | | |
| propylene glycol | 20.00% | 20.00% | 20.00% | 20.00% | 20.00% | 20.00% |
| cineole | 1.00% | 1.00% | 1.00% | 1.00% | 1.00% | 1.00% |
| Sodium | 0.20% | — | — | — | — | — |
| Metabisulfite | | | | | | |
| Sodium | — | 0.20% | — | — | — | — |
| Thiosulfate | | | | | | |
| Potassium | — | — | 0.05% | — | — | — |
| thiosulfate | | | | | | |
| Ammonium | — | — | — | 0.20% | — | — |
| Thiosulfate | | | | | | |
| Sodium Sulfate | — | — | — | — | 0.20% | — |

The compositions were prepared as follows: The ethanol (anhydrous), propylene glycol, 1,8-cineole, and the antioxidant or ethanol (anhydrous) as defined in Table 10 were mixed until well dispersed. Magnesium chloride (anhydrous) and minocycline hydrochloride were added to the mixture and mixed until clear. Hydroxypropyl cellulose (KLUCEL HF, Ashland Specialty Chemical, Covington, Ky.) was added slowly and mixed until clear and uniformed.

Table 23 describes the measured relative concentration of 4-epi-minocycline and relative concentration of active minocycline within each composition. Measurements were taken at baseline and after 1-, 2-, and 4-weeks of aging at 40° C. in closed glass vials. The computer program Microsoft excel (Microsoft, Redmond, Wash.) was used to evaluate the change in concentration per week based on the measured data for each composition to obtain a linear least squares best fit equation for each composition. The slope of this linear least squares best fit equation is presented in Table 23 as the rate of change in concentration over the 4-week aging period. Table 23 describes the baseline measurement and the best fit slope for the change per week.

TABLE 23
Relative Concentrations of 4-Epi-Minocycliine and Active Minocycline for
4-week Aging Study at 40° C.

| | | Best fit | | Best fit | Significant |
|---|---|---|---|---|---|
| | | relative | | relative | color |
| | Baseline | concentration | Baseline | concentration | change |
| | relative | of 4-epi- | relative | of active | after 4 |
| Composition | concentration | minocycline | concentration | minocycline | weeks at |
| Number | of 4-epi- | increase per | of active | decrease per | 40° C. in |
| (see | minocycline | week at 40° C. | minocycline | week at 40° C. | closed |
| Table 22) | (%) | (%/week) | (%) | (%/week) | glass vials |
| 1-sbs | 0.87 | 0.294 | 97.96 | 0.271 | No |
| 27-st.2 | 0.88 | 0.242 | 97.72 | 0.268 | No |
| 52-pt | 0.93 | 0.830 | 97.79 | 0.729 | No |
| 53-at | 0.91 | 0.321 | 97.59 | 0.326 | No |
| 51-ss | 0.90 | 0.986 | 97.99 | 1.075 | Yes |
| 19-Ctrl | 0.85 | 0.854 | 98.12 | 0.960 | Yes |

Preferred compositions show a low baseline 4-epi-minocycline relative concentration and a small or no increase in 4-epi-minocycline relative concentration per week. For example, in some preferred compositions, the relative concentration of 4-epi-minocycline is less than 1.0% at a baseline measurement and increases less than 1.00% per week when measured over a 4-week period at 40° C. in closed glass vials, wherein the 4-week period starts immediately after the baseline measurement. More preferably, the relative concentration of 4-epi-minocycline is less than 1.0% at a baseline measurement and increases less than 0.3% per week when measured over a 4-week period at 40° C. in closed glass vials, wherein the 4-week period starts immediately after the baseline measurement. Preferably, the relative concentration of 4-epi-minocycline is in the range of about 0.50% to about 1.00% at a baseline measurement and increases at a rate in the range of about 0.20% to about 0.40% per week when measured over a 4-week period at 40° C. in closed glass vials, wherein the 4-week period starts immediately after the baseline measurement.

Preferred compositions show a high active minocycline relative concentration and a small or no decrease in active minocycline relative concentration per week. For example, in some preferred compositions, the relative concentration of active minocycline is at least 97.0% at a baseline measurement and decreases less than 0.8% per week when measured over a 4-week period at 40° C. in closed glass vials, wherein the 4-week period starts immediately after the baseline measurement. More preferably, the relative concentration of 4-epi-minocycline is at least 97.5% at a baseline measurement and decreases less than 0.3% per week when measured over a 4-week period at 40° C. in closed glass vials, wherein the 4-week period starts immediately after the baseline measurement. Preferably, the relative concentration of 4-epi-minocycline is in the range of about 97.0% to about 99.0% at a baseline measurement and decreases at a rate in the range of about 0.27% to about 0.73% per week when measured over a 4-week period at 40° C. in closed glass vials, wherein the 4-week period starts immediately after the baseline measurement.

This study further evaluated the difference in color between the compositions at baseline and after aging for 4 weeks at 40° C. in closed glass vials. The four compositions that included a sulfite salt or a thiosulfate salt as an antioxidant, i.e., compositions 1-sbs, 27-st.2, 52-pt, and 53-at, did not have a significant difference in color between the aged and baseline compositions. Each of the other no antioxidant added compositions showed significant color differences (51-ss, and 19-Ctrl). Preferred compositions show no significant color changes after aging for 4 weeks at 40° C. in closed glass vials.

### Example 17

### Stability of Minocycline in Exemplary Compositions

A study was performed in which samples comprising minocycline were dissolved in the compositions as described in Table 24. The degradation and stability of minocycline in compositions were evaluated at baseline and after storage for 1, 2, 4, 8 and 14 weeks in the dark at 40° C. within sealed glass vials.

The first six materials, i.e., minocycline hydrochloride, hydroxypropyl cellulose, magnesium chloride (anhydrous), ethanol (anhydrous), propylene glycol, and 1,8-cineole make up 99.7-99.8% of each of the evaluated compositions. The remaining 0.2-0.3% consisted of one or combination of two antioxidants listed in Table 24. Composition with 1% minocycline were tested in composition 1-sbs (1%), 27-st.2 (1%), 48-comb 8.2 (1%); and 2% Minocycline are tested in composition 1-sbs (2%), 27-st.2 (2%), 48-comb 8.2 (2%).

Table 25 describes the measured relative concentration of 4-epi-minocycline and relative concentration of active minocycline within each composition. Measurements were taken at baseline and after 1-, 2-, 4-, 8-, and 14-weeks of aging at 40° C. in closed glass vials. The computer program Microsoft excel (Microsoft, Redmond, Wash.) was used to evaluate the change in concentration per week based on the measured data for each composition to obtain a linear least squares best fit equation for each composition. The slope of this linear least squares best fit equation is presented in Table 25 as the rate of change in concentration over the 4-week aging period. Table 25 describes the baseline measurement and the best fit slope for the change per week.

TABLE 24
Exemplary Compositions for Evaluating Stability of Minocycline (w/w)

| | | | 27- | 27- | 48- | 48- |
|---|---|---|---|---|---|---|
| Composition | 1-sbs | 1-sbs | st.2 | st.2 | comb | comb |
| Component | (1%) | (2%) | (1%) | (2%) | 8.2 (1%) | 8.2 (2%) |
| minocycline | 1.18% | 2.36% | 1.18% | 2.36% | 1.18% | 2.36% |
| hydrochloride | | | | | | |
| hydroxypropyl | 0.60% | 0.60% | 0.60% | 0.60% | 0.60% | 0.60% |
| cellulose HF | | | | | | |
| magnesium | 1.20% | 2.30% | 1.20% | 2.30% | 1.20% | 2.30% |
| chloride, | | | | | | |
| anhydrous | | | | | | |
| ethanol, | 75.82% | 73.54% | 75.82% | 73.54% | 75.72% | 73.44% |
| anhydrous | | | | | | |
| propylene | 20.00% | 20.00% | 20.00% | 20.00% | 20.00% | 20.00% |
| glycol | | | | | | |
| cineole | 1.00% | 1.00% | 1.00% | 1.00% | 1.00% | 1.00% |
| Sodium | 0.20% | 0.20% | — | — | 0.20% | 0.20% |
| Metabisulfite | | | | | | |
| Sodium | — | — | 0.20% | 0.20% | 0.10% | 0.10% |
| Thiosulfate | | | | | | |

TABLE 25
Relative Concentrations of 4-Epi-Minocycline and Active Minocycline
for 3-Month Aging Study at 40° C.

| | | Best fit | | Best fit | Significant |
|---|---|---|---|---|---|
| | | relative | | relative | color |
| | Baseline | concentration | Baseline | concentration | change |
| | relative | of 4-epi- | relative | of active | after 4 |
| Composition | concentration | minocycline | concentration | minocycline | weeks at |
| Number | of 4-epi- | increase per | of active | decrease per | 40° C. in |
| (see | minocycline | week at 40° C. | minocycline | week at 40° C. | closed |
| Table 12) | (%) | (%/week) | (%) | (%/week) | glass vials |
| 1-sbs (1%) | 0.87 | 0.284 | 98.01 | 0.301 | No |
| 27-st.2 (1%) | 0.88 | 0.241 | 97.72 | 0.267 | No |
| 48-comb 8.2 | 0.86 | 0.249 | 97.63 | 0.331 | No |
| (1%) | | | | | |
| 1-sbs (2%) | 0.85 | 0.477 | 98.13 | 0.468 | No |
| 27-st.2 (2%) | 0.86 | 0.435 | 97.84 | 0.413 | No |
| 48-comb 8.2 | 0.90 | 0.424 | 97.71 | 0.410 | No |
| (2%) | | | | | |

Preferred compositions show a low baseline 4-epi-minocycline relative concentration and a small or no increase in 4-epi-minocycline relative concentration per week. Preferred compositions also show a high active minocycline relative concentration and a small or no decrease in active minocycline relative concentration per week. Composition containing thiosulfate (27-st.2 (1%) and 27-st.2 (2%)) or combination of sulfite and thiosulfate (48-comb 8.2 (1%) and (48-comb 8.2 (2%) show improved stability than composition containing sulfite antioxidant alone (1-sbs (1%) and 1-sbs (2%)) for both tetracycline drug strengths at 1% and 2%.

### Example 18

### Stability of Minocycline in Exemplary Compositions

A study was performed in which samples comprising minocycline were dissolved in the compositions as described in Table 26 to evaluate the selected stabilizers on drug stability and epimer formation. Table 27 describes the main stabilizer tested in this study. Composition 1-sbs contains sodium metabisulfite (SMBS) as the source of antioxidant and magnesium chloride as the source of divalent ions. Composition 52-MgS contains magnesium sulfite as the source for both antioxidant and divalent ions. Composition 64-MgS+Mg contains magnesium sulfite as the source for both antioxidant and divalent ions, and additional magnesium chloride as the source of divalent ions. Composition 65-MgTS contains magnesium thiosulfate hexahydrate as the source for both antioxidant and divalent ions. Composition 66-Ctrl 3 contains no antioxidant and divalent ions as the negative control. These samples were stored in sealed amber glass containers for up to 2 months at 40° C.

Degradation of minocycline to its epimer was quantified by evaluating the change in the relative concentration of 4-epi-minocycline, which was calculated as the 4-epi-minocycline peak area divided by the active minocycline peak area. As a separate quantification, stability was quantified by evaluating the change in the relative concentration of active minocycline, which was calculated as the active minocycline peak divided by the sum of the peak areas for all peaks observed in the HPLC chromatograph. For these measurements, a runtime of 20 minutes was used for the HPLC

TABLE 26
Exemplary Compositions for Evaluating
Stability of Minocycline (w/w)

| Composition | 1- | 52- | 64- | 65- | 66- |
|---|---|---|---|---|---|
| Component | sbs | MgS | MgS + Mg | MgTS | Ctrl 3 |
| minocycline | 1.20% | 1.20% | 1.20% | 1.20% | 1.20% |
| hydrochloride | | | | | |
| hydroxypropyl | 0.60% | 0.60% | 0.60% | 0.60% | 0.60% |
| cellulose HF | | | | | |
| magnesium | 1.20% | — | 1.20% | — | — |
| chloride, | | | | | |
| anhydrous | | | | | |
| ethanol, anhydrous | 75.80% | 77.00% | 75.80% | 74.12% | 77.20% |
| propylene glycol | 20.00% | 20.00% | 20.00% | 20.00% | 20.00% |
| cineole | 1.00% | 1.00% | 1.00% | 1.00% | 1.00% |
| Sodium | 0.20% | — | — | — | — |
| Metabisulfite | | | | | |
| Magnesium Sulfite | — | 0.20% | 0.20% | — | — |
| Magnesium | — | — | — | 3.08% | — |
| thiosulfate | | | | | |
| hexahydrate | | | | | |

TABLE 27
Stabilizers for Stability Studies

| Composition Number | Main Components |
|---|---|
| 1-sbs | MNC, MgCl₂, SMBS |
| 52-MgS | MNC, MgSO₃ |
| 64-MgS + Mg | MNC, MgSO₃, MgCl2 |
| 65-MgTS | MNC, MgS₂O₃ 6H₂O |
| 66-Ctrl 3 | MNC |

Table 28 describes the measured relative concentration of 4-epi-minocycline and relative concentration of active minocycline within each composition. Measurements were taken at baseline and after 2 months of aging at 40° C. in closed glass vials.

Preferred compositions show a low baseline 4-epi-minocycline relative concentration and a small or no increase in 4-epi-minocycline relative concentration after 2 months at 40° C. Preferred compositions also show a high active minocycline relative concentration and a small or no decrease in active minocycline relative concentration after 2 months at 40° C. Preferred compositions show no significant color changes after aging for 2 months at 40° C. in closed glass vials.

The minocycline with sodium metabisulfite (SMBS) as the source of antioxidant and magnesium chloride as the source of divalent ions in composition 1-sbs degraded by approximately 1.5% and had 1.2% epimer increase over the 2-month period. The minocycline without both antioxidant and divalent ions in composition 66-Ctrl 3 degraded by over 50% and approximately 90% epimer increase over the 2-month period as the negative control. The minocycline with magnesium sulfite as the source for both antioxidant and divalent ions in composition 52-MgS degraded by approximately close to 8% and had 8% epimer increase over the 2-month period, indicates magnesium sulfite as an effective stabilizer. The minocycline in composition 64-MgS+Mg, magnesium sulfite as the source for both antioxidant and divalent ions, and additional magnesium chloride as the source of divalent ions, had less degradation than composition 1-sbs, which indicates that the combination of magnesium sulfite and magnesium chloride makes minocycline in ethanol more stable. The minocycline in composition 65-MgTS, magnesium sulfite as the source for both antioxidant and divalent ions, and additional magnesium chloride as the source of divalent ions, had less degradation than composition 1-sbs, which indicates that the combination of magnesium sulfite and magnesium chloride makes minocycline in ethanol more stable. The minocycline in composition 65-MgTS had slightly improvement of stability than the negative control in formulation color and minocycline peak area change.

TABLE 28
Relative Concentrations of 4-Epi-Minocycline and Active Minocycline
for 2-Month Aging Study at 40° C.

| | | Relative | | | Significant |
|---|---|---|---|---|---|
| | Baseline | concen- | Baseline | | color |
| | relative | tration | relative | Minocycline | change |
| | concen- | of 4-epi.- | concen- | concen- | after 4 |
| | tration | mino- | tration | tration | weeks at |
| | of 4-epi- | cycline | of active | (% peak | 40° C. in |
| Composition | mino- | after 2 | mino- | area) after 2 | closed |
| Number (see | cycline | months at | cycline | months at | glass |
| Table 14) | (%) | 40° C. | (%) | 40° C. | vials |
| 1-sbs | 0.92 | 2.13 | 97.96 | 96.51 | No |
| 52-MgS | 1.38 | 9.48 | 97.40 | 89.44 | No |
| 64-MgS + | 0.91 | 1.73 | 97.71 | 96.69 | No |
| Mg | | | | | |
| 65-MgTS | 1.95 | 96.52 | 96.64 | 47.67 | Yes |
| 66-Ctrl 3 | 2.99 | 92.51 | 96.16 | 45.29 | Most |
| | | | | | Color |
| | | | | | Change |

### Example 19

### In Vivo Skin Penetration

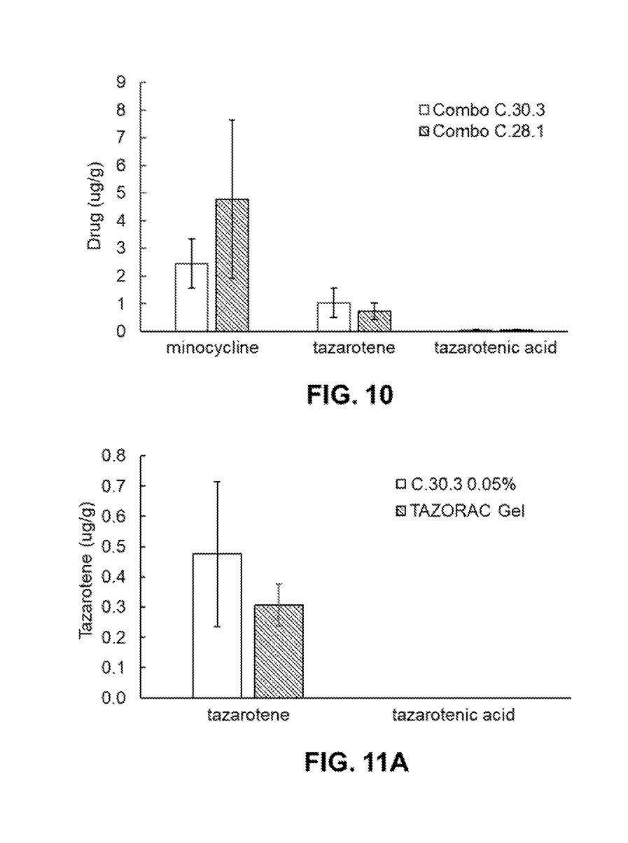

Penetration experiments were conducted in Sprague Dawley rats to determine whether minocycline and/or tazarotene penetrated into the skin in vivo. Sprague Dawley rats (6-8 wks old, n=3 rats per treatment group) were shaved to create 5 cm² patches on the dorsal side. A dose volume ˜2.5 mg/cm² of the compositions identified as Combo C.30.3 and Combo C.28.1 (see Example 9, Table 13-1) were applied once daily to the patch for 14 days. After 14 days, the patch skin was biopsied and minocycline and/or tazarotene and its metabolite, tazarotenic acid, were extracted using acidified methanol. The supernatants were analyzed by high performance liquid chromatography. The amount of drug is presented in FIG. 10.

### Example 20

### In Vivo Skin Penetration

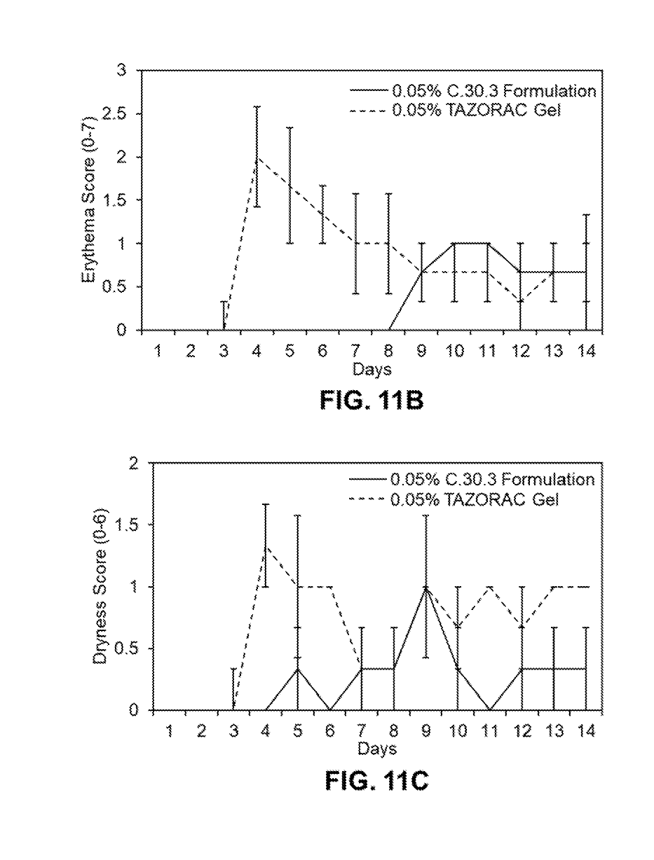

Penetration and erythema/dryness experiments were conducted in Sprague Dawley rats to compare the penetration and erythema/dryness potential of tazarotene in an exemplary composition to a commercially marketed topical tazarotene composition (TAZORAC gel 0.05%) in vivo. The tazarotene composition used in the study is identified in Table 13-1 as Combo C.30.3. The penetration comparison follows the in vivo method described in Example 19. The erythema/dryness observations of the treated skin patch were graded daily for 14 days based upon a modified Draize scoring system. The amount of drug is presented in FIGS. 11A-11C.

The amount of tazarotene that was measured within the skin samples for the Combo C.30.3 exemplary composition and the commercially marketed composition, is presented in FIG. 11A. As shown in FIG. 11A, tazarotene in the exemplary composition delivers more tazarotene on average compared to the commercially marketed composition. Thus, this experiment showed that the exemplary composition may be more efficient in tazarotene delivery into the rat skin in vivo.

The erythema and dryness scores measured daily at the treatment site is presented in FIGS. 11B-11C. As shown in FIG. 11B, the exemplary composition caused less erythema overall and did not show an initial reaction to the tazarotene as the commercially marketed composition displayed. Similarly, as shown in FIG. 11C, the exemplary composition caused less dryness overall and did not show an initial reaction compared to the commercially marketed composition. Thus, tazarotene in the exemplary composition showed less erythema and dryness of the rat skin in vivo compared to the commercially marketed composition (TAZORAC gel 0.05%).

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. A topical composition, comprising:
a drug selected from a selective retinoid, a tetracycline-class drug, and a combination of a selective retinoid and a tetracycline-class drug,
a monohydric aliphatic alcohol, and
a carboxylate ester with a ratio of ester groups to carbon atoms of at least 0.05.

2. The topical composition of claim 1, wherein the drug is dissolved in the composition.

3. The topical composition of claim 1, further comprising a salt of a divalent cation.

4. The topical composition of claim 1, further comprising an antioxidant selected from a sulfite, a thiosulfate, and a combination thereof.

5. The topical composition of claim 4, wherein the antioxidant is a divalent cation.

6. The topical composition of claim 5, wherein the antioxidant with a divalent cation is magnesium sulfite, magnesium thiosulfate, or a combination thereof.

7. A topical composition, comprising:
a selective retinoid, a tetracycline-class drug, or both a selective retinoid and a tetracycline-class drug,
a monohydric aliphatic alcohol,
a salt of a divalent cation, and
an antioxidant.

8. The topical composition of claim 7, wherein the molar ratio of the divalent cation to the tetracycline-class drug in the topical composition is at least about 4:1.

9. The topical composition claim 7, further comprising a carboxylate ester with a ratio of ester groups to carbon atoms of at least 0.05.

10. The topical composition of claim 9, wherein the concentration of the carboxylate ester in the topical composition is between about 1-30% by weight.

11. The topical composition of claim 9, wherein the carboxylate ester is selected from the group consisting of isopropyl myristate, medium-chain triglycerides, diisopropyl adipate, ethyl acetate, triacetin, dimethyl succinate, propyl acetate, and combinations thereof.

12. The topical composition of claim 7, wherein the salt of a divalent cation is a magnesium salt.

13. The topical composition of claim 12, wherein the magnesium salt is magnesium chloride, magnesium sulfite, or magnesium thiosulfate.

14. The topical composition of claim 7, wherein the monohydric aliphatic alcohol is selected from the group consisting of ethanol, isopropanol, propyl alcohol, tert-butyl alcohol, and combinations thereof.

15. The topical composition of claim 14, wherein the concentration of the monohydric aliphatic alcohol in the topical composition is between about 50-99% by weight.

16. The topical composition of claim 7, wherein the selective retinoid is dissolved in the composition.

17. The topical composition of claim 16, wherein the tetracycline class drug is dissolved in the composition.

18. The topical composition of claim 7, wherein the tetracycline-class drug is minocycline or doxycycline.

19. The topical composition of claim 7, wherein the selective retinoid is tazarotene or adapalene.

20. The topical composition of claim 7, further comprising a polyol.

21. The topical composition of claim 20, wherein the polyol is a C3-C8 diol or a triol.

22. The topical composition of claim 20, wherein the polyol is selected from the group consisting of propylene glycol, glycerol and glycerin.

23. The topical composition of claim 20, wherein the concentration of the polyol in the topical composition is between about 2-40% by weight.

24. The topical composition of claim 7, further comprising a sulfite, a thiosulfate, or both.

25. The topical composition of claim 24, wherein the sulfite is sodium bisulfite, sodium metabisulfite, magnesium sulfite, or a combination thereof, and the thiosulfate is sodium thiosulfate, ammonium thiosulfate, magnesium thiosulfate or a combination thereof.

26. A method for treatment of a dermatological condition or disease, comprising:
applying the topical composition of claim 1 to an exterior epithelial surface of a mammalian body at least once daily for a period of at least 4 weeks.

27. The method of claim 26, wherein the dermatological condition or disease is selected from the group consisting of acne, rosacea, and psoriasis.

28. The method of claim 26, wherein the dermatological condition or disease is an orphan disease selected from Hidradenitis suppurativa, Hailey Hailey disease, and Darier's disease.

29. A method for treatment of a dermatological condition or disease, comprising:
applying the topical composition of claim 7 to an exterior epithelial surface of a mammalian body at least once daily for a period of at least 4 weeks.

30. The method of claim 29, wherein the dermatological condition or disease is selected from the group consisting of acne, rosacea, and psoriasis.

31. The method of claim 29, wherein the dermatological condition or disease is an orphan disease selected from Hidradenitis suppurativa, Hailey Hailey disease, and Darier's disease.
